(19) United States
(12) Patent Application Publication
(10) Pub. No.: US 2007/0099251 A1
(43) Pub. Date: May 3, 2007

(54) Tissue-and serum-derived glycoproteins and methods of their use

(75) Inventors: Hui Zhang, Seattle, WA (US); Rudolf H. Aebersold, Zurich (CH)

Correspondence Address: SEED INTELLECTUAL PROPERTY LAW GROUP PLLC, 701 FIFTH AVE, SUITE 5400, SEATTLE, WA, 98104, US

(73) Assignee: Institute for Systems Biology, Seattle, WA (US)

(21) Appl. No.: 11/582,861

(22) Filed: Oct. 17, 2006

Related U.S. Application Data
(60) Provisional application No. 60/728,044, filed on Oct. 17, 2005.

(51) Int. Cl. G01N 33/574; C12M 3/00

(52) U.S. Cl. 435/7.23; 435/7.92; 435/287.2; 977/902

(57) ABSTRACT

The present invention is directed generally to tissue-derived glycoproteins and glycosites detectable in plasma via mass spectrometric analysis of glycoproteins from both tissues and blood. The invention also provides methods for identifying tissue-derived glycoproteins and glycosites in plasma, panels of detection reagents for detecting same, as well methods for detecting disease using such panels. The invention further provides a database of tissue-derived glycoproteins and glycosites detectable in plasma.

### STATEMENT OF GOVERNMENT INTEREST

This invention was made with government support in part with federal funds from the National Heart, Lung, and Blood Institute, National Institutes of Health, under contract No. N01-HV-28179, with federal funds from the National Cancer Institute, National Institutes of Health, by grant R21-CA-114852 and U01-CA-111244, and under contract No. N01-CO-12400, and by NIH grant R01-AI-41109-01. The government may have certain rights in this invention.

### STATEMENT REGARDING TABLES SUBMITTED ON CD-ROM

Tables 1A and 1B associated with this application are provided on CD-ROM in lieu of a paper copy, and are hereby incorporated by reference into the specification. Two CD-ROMs are provided, containing identical copies of the tables, which are designed to be viewed in landscape presentation: CD-ROM No. 1 is labeled Copy 1, contains the 2 table files which are 2.06 MB combined and created on Oct. 17, 2006; CD-ROM No. 2 is labeled Copy 2, contains the 2 table files which are 2.06 MB combined and created on Oct. 17, 2006.

LENGTHY TABLES FILED ON CD
The patent application contains a lengthy table section. A copy of the table is available in electronic form from the USPTO web site (http://seqdata.uspto.gov/?pageRequest=docDetail&DocID=US20070099251A1). An electronic copy of the table will also be available from the USPTO upon request and payment of the fee set forth in 37 CFR 1.19(b)(3).

### STATEMENT REGARDING SEQUENCE LISTING SUBMITTED ON CD-ROM

The Sequence Listing associated with this application is provided on CD-ROM in lieu of a paper copy, and is hereby incorporated by reference into the specification. Three CD-ROMs are provided, containing identical copies of the sequence listing: CD-ROM No. 1 is labeled COPY 1, contains the file 404.app.txt which is 57.9 MB and created on Oct. 17, 2006; CD-ROM No. 2 is labeled COPY 2, contains the file 404.app.txt which is 57.9 MB and created on Oct. 17, 2006; CD-ROM No. 3 is labeled CRF (Computer Readable Form), contains the file 404.app.txt which is 57.9 MB and created on Oct. 17, 2006.

### BACKGROUND OF THE INVENTION

1. Field of the Invention

The present invention is directed generally to tissue- and serum-derived glycoproteins and glycosites identified via mass spectrometric analysis of glycoproteins from both tissues and blood. The invention also provides methods for identifying tissue- and serum-derived glycoproteins and glycosites, panels of detection reagents for detecting same, as well methods for detecting disease using such panels. The invention further provides a database of tissue-, plasma- and serum-derived glycoproteins and glycosites.

2. Description of the Related Art

Biomarker detection can have a tremendous impact on the clinical outcomes of patients. A particular challenge in the diagnosis and treatment of human disease is the identification of molecular markers for detection of disease at an early and treatable stage, and the molecular definition of disease progression to allow for implementation of the most effective treatment (1). Expression array studies have shown that such markers, or marker panels, exist in cells from disease tissues and can be associated with pathological changes in the disease and its various prognoses (2, 3). Unfortunately, most tissues are not readily accessible for routine screening. Thus expression array studies are limited to general screening for diagnosis of disease.

On the other hand, blood has long been thought as a window to a person's health. The basis behind this idea is that blood picks up molecular cues as it circulates throughout the body and that these cues, or biomarkers, can collectively inform about the various organs, tissues or cell type from which they originated. It thus follows that if tissue-specific changes or patterns can be detected in blood, then the development of simple blood-tests could allow for routine diagnostic screening. However, the discovery of tissue-specific changes in blood is hampered by the fact that human blood is extremely complex, consisting of minimally tens of thousands of different molecular species that span a concentration range of at least 10 orders of magnitude (4). Indeed, the plasma proteome is dominated by 22 abundant proteins that constitute 99% of the total protein mass (5). Many of these abundant plasma proteins are altered by mutations, alternative splicing, post-translational modifications such as phosphorylation, glycosylation, acetylation, methionine oxidation, protease processing, and other mechanisms, resulting in multiple forms for each protein. It has been estimated that one protein may generate on the order of 100 species (4, 6). Immunoglobulin alone contains thousands of, if not millions of, different molecular species. As a result, it is difficult to penetrate these high abundance plasma proteins to detect low abundance proteins using current high-throughput proteomic approaches, such as two dimensional electrophoresis (2DE) or mass spectrometry-based methods. While many of these abundant plasma proteins are indicators of interesting biology, and have been reported to change in abundance in response to certain types of diseases (7), they are unlikely to be useful as markers for specific disease states. Further, the ability to extend these techniques to easy, consistent, and high throughput diagnostic assays has been extremely limited. Thus, there is a need in the art to provide such diagnostic assays. The present invention provides for methods and assays that fulfill these and other needs.

### BRIEF SUMMARY OF THE INVENTION

One aspect of the present invention provides a diagnostic panel comprising a plurality of detection reagents wherein each detection reagent is specific for one tissue-derived serum glycoprotein; wherein the tissue-derived serum glycoproteins detected by the plurality of detection reagents are derived from the same tissue and selected from the tissue-derived serum glycoprotein sets provided in Table 1. In further embodiments, the plurality of detection reagents is selected such that the level of at least two, three, four, five, six, seven, or more of the tissue-derived serum glycoproteins detected by the plurality of detection reagents in a blood sample from a subject afflicted with a disease affecting a tissue from which the tissue-derived serum glycoproteins are derived is above or below a predetermined normal range. In certain embodiments, the disease affects the prostate and the tissue-derived serum glycoproteins detected by the plurality of detection reagents are selected from the prostate-derived serum glycoproteins listed in Table 1. In yet another embodiment, the plurality of detection reagents detect two, three, four, five, six, seven, eight, nine, ten, or more of the prostate-derived serum glycoproteins listed in Table 1. In certain embodiments, the plurality of detection reagents detect two or more prostate-derived serum glycoproteins selected from the group consisting of PSA, CD13, CD14, CD26, CD44, CD45, CD56, CD90, CD91, CD107a, CD107b, CD109, CD166, CD143, CD224, PSMA-1, Glutamate carboxypeptidase II, MAC-2 binding protein, metalloproteinase inhibitor 1, and tumor endothelial marker 7-related precursor.

In another embodiment, the plurality of detection reagents is between two and 100 detection reagents. Thus, the panels of the present invention can have 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, or more detection reagents thereon. In certain embodiments the panels of the present invention may have 100, 110, 120, 130, 140, 150, 160, 170, 180, 190, 200, or more detection reagents thereon.

In a further embodiment, the panels of the invention further comprise one or more detection reagents that are each specific for a prostate-derived glycoprotein listed in Table 1 that does not overlap with the plasma-derived glycoproteins listed in Table 1.

In another embodiment, the disease affects the bladder and the tissue-derived serum glycoproteins detected by the plurality of detection reagents are selected from the bladder-derived serum glycoproteins listed in Table 1. In a related embodiment, the plurality of detection reagents detect two, three, four, five, six, seven, eight, nine, ten, or more of the bladder-derived serum glycoproteins listed in Table 1. Further, the diagnostic panel may comprise one or more detection reagents that are each specific for a bladder-derived glycoprotein listed in Table 1 that does not overlap with the plasma-derived glycoproteins listed in Table 1.

In another embodiment, the diagnostic panel comprises detection reagents for the detection of a disease that affects the liver and the tissue-derived serum glycoproteins detected by the plurality of detection reagents are selected from the liver-derived serum glycoproteins listed in Table 1. In this regard, in certain embodiments, the plurality of detection reagents detect two, three, four, five, six, seven, eight, nine, ten, or more of the liver-derived serum glycoproteins listed in Table 1. In another embodiment, the d further comprising one or more detection reagents that are each specific for a liver-derived glycoprotein listed in Table 1 that does not overlap with the plasma-derived glycoproteins listed in Table 1.

In another embodiment, the diagnostic panel comprises detection reagents for the detection of a disease that affects the breast and the tissue-derived serum glycoproteins detected by the plurality of detection reagents are selected from the breast-derived serum glycoproteins listed in Table 1. In a related embodiment, the plurality of detection reagents detect two, three, four, five, six, seven, eight, nine, ten, or more of the breast-derived serum glycoproteins listed in Table 1. In certain embodiments, the plurality of detection reagents detect two or more breast-derived serum glycoproteins selected from the group consisting of CD71, CD98, CD107b, CD155, CD224, MAC-2 binding protein, receptor protein-tyrosine kinase erbB-2, and tumor-associated calcium signal transducer 2. In one embodiment, the panels of the present invention further comprise one or more detection reagents that are each specific for a breast-derived glycoprotein listed in Table 1 that does not overlap with the plasma-derived glycoproteins listed in Table 1.

In another embodiment, the diagnostic panel comprises detection reagents for the detection of a disease that affects lymphocytes and the tissue-derived serum glycoproteins detected by the plurality of detection reagents are selected from the lymphocyte-derived serum glycoproteins listed in Table 1. In a further embodiment, the plurality of detection reagents detect two, three, four, five, six, seven, eight, nine, ten, or more of the lymphocyte-derived serum glycoproteins listed in Table 1. In certain embodiments, the panel further comprises one or more detection reagents that are each specific for a lymphocyte-derived glycoprotein listed in Table 1 that does not overlap with the plasma-derived glycoproteins listed in Table 1.

In another embodiment, the diagnostic panel comprises detection reagents for the detection of a disease that affects the ovary and the tissue-derived serum glycoproteins detected by the plurality of detection reagents are selected from the ovary-derived serum glycoproteins listed in Table 1. In yet a further embodiment, the plurality of detection reagents detect two, three, four, five, six, seven, eight, nine, ten, or more of the ovary-derived serum glycoproteins listed in Table 1. In a related embodiment, the panel may further comprise one or more detection reagents that are each specific for a ovary-derived glycoprotein listed in Table 1 that does not overlap with the plasma-derived glycoproteins listed in Table 1.

Another aspect of the invention provides a diagnostic panel comprising a plurality of detection reagents wherein each detection reagent is specific for one tissue-derived serum glycoprotein; wherein the tissue-derived serum glycoproteins detected by the plurality of detection reagents are selected from two or more of the tissue-derived serum glycoprotein sets provided in Table 1. In one embodiment, the plurality of detection reagents is selected such that the level of at least two, three, four, five, six, seven, eight, nine, ten, or more of the tissue-derived serum glycoproteins detected by the plurality of detection reagents in a blood sample from a subject afflicted with a disease affecting the organs from which the tissue-derived serum glycoproteins are derived is above or below a predetermined normal range. In one embodiment, the plurality of detection reagents is between two and 100 detection reagents. Thus, the panels of the present invention can have 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, or more detection reagents thereon. In certain embodiments the panels of the present invention may have 100, 110, 120, 130, 140, 150, 160, 170, 180, 190, 200, or more detection reagents thereon.

In certain embodiments, the detection reagent comprises an antibody or an antigen-binding fragment thereof, a DNA or RNA aptamer, or an isotope labeled peptide, or a combination of any of these detection reagents.

A further aspect of the invention provides a method for defining a biological state of a subject comprising a) measuring the level of at least two tissue-derived serum glycoproteins selected from any one of the tissue-derived serum glycoprotein sets provided in Table 1 in a blood sample from the subject; b) comparing the level determined in (a) to a predetermined normal level of the at least two tissue-derived serum glycoproteins; wherein the measured level of at least one of the two tissue-derived serum glycoproteins is above or below the predetermined normal level and wherein said measured level defines the biological state of the subject. In certain embodiments, the level of the at least two tissue-derived serum glycoproteins is measured using an immunoassay. In this regard, the immunoassay may be an ELISA or other immunoassay known in the art. In another embodiment, the at least two tissue-derived serum glycoproteins is measured using mass spectrometry or an aptamer capture assay.

A further aspect of the invention provides a method for defining a biological state of a subject comprising; a) measuring the level of at least two tissue-derived serum glycoproteins selected from any two or more of the tissue-derived serum glycoprotein sets provided in Table 1; b) comparing the level determined in (a) to a predetermined normal level of the at least two tissue-derived serum glycoproteins; wherein the measured level of at least one of the two tissue-derived serum glycoproteins is above or below the predetermined normal level and wherein said measured level defines the biological state of the subject. In some embodiments, the at least two tissue-derived serum glycoproteins is measured using an immunoassay such as an ELISA, or they can be measured using any of a variety of methods known in the art, such as mass spectrometry or an aptamer capture assay.

Another aspect of the invention provides a method for defining a disease-associated tissue-derived blood fingerprint comprising; a) measuring the level of at least two tissue-derived serum glycoproteins selected from any one of the tissue-derived serum glycoprotein sets provided in Table 1 in a blood sample from a subject determined to have a disease affecting the tissue from which the at least two tissue-derived serum glycoproteins are selected; b) comparing the level of the at least two tissue-derived serum glycoproteins determined in (a) to a predetermined normal level of the at least two tissue-derived serum glycoproteins; wherein the measured level of at least one of the at least two tissue-derived serum glycoproteins in the blood sample from the subject determined to have the disease is below or above the corresponding predetermined normal level and wherein said measured level defines the disease-associated tissue-derived blood fingerprint. In certain embodiments, step (a) comprises measuring the level of at least three, four, five, six, seven, eight, nine, ten, or more tissue-derived serum glycoproteins selected from any one of the tissue-derived serum glycoprotein sets provided in Table 1 and wherein the measured level of at least two, three, four, five, six, seven, eight, nine, ten, or more of the at least three tissue-derived serum glycoproteins in the blood sample from the subject determined to have the disease is below or above the corresponding predetermined normal level and wherein said measured level defines the disease-associated tissue-derived blood fingerprint. In certain embodiments, the level of the at least two tissue-derived serum glycoproteins is measured using antibodies or antigen-binding fragments thereof specific for each protein. The antibodies may be monoclonal antibodies. In other embodiments, the level of the at least two tissue-derived serum glycoproteins is measured using mass spectrometry, an aptamer capture assay, or other assays known in the art. In certain embodiments, the disease is prostate cancer and the at least two tissue-derived serum glycoproteins are selected from the prostate-derived serum glycoproteins listed in Table 1. In a further embodiment, the disease is breast cancer and the at least two tissue-derived serum glycoproteins are selected from the breast-derived serum glycoproteins listed in Table 1. In yet another embodiment, the disease is bladder cancer and the at least two tissue-derived serum glycoproteins are selected from the bladder-derived serum glycoproteins listed in Table 1. In a further embodiment, the disease is liver cancer and the at least two tissue-derived serum glycoproteins are selected from the liver-derived serum glycoproteins listed in Table 1.

Another aspect of the invention provides a method for defining a disease-associated tissue-derived blood fingerprint comprising; a) measuring the level of at least two tissue-derived serum glycoproteins selected from two or more of the tissue-derived serum glycoprotein sets provided in Table 1 in a blood sample from a subject determined to have a disease of interest; b) comparing the level of the at least two tissue-derived serum glycoproteins determined in (a) to a predetermined normal level of the at least two tissue-derived serum glycoproteins; wherein a level of at least one of the at least two tissue-derived serum glycoproteins in the blood sample from the subject determined to have the disease that is below or above the corresponding predetermined normal level defines the disease-associated tissue-derived blood fingerprint. In one embodiment, step (a) comprises measuring the level of at least three tissue-derived serum glycoproteins selected from two or more of the tissue-derived serum glycoprotein sets provided in Table 1 and wherein a level of at least two of the at least three tissue-derived serum glycoproteins in the blood sample from the subject determined to have the disease that is below or above the corresponding predetermined normal level defining the disease-associated tissue-derived blood fingerprint. In a further embodiment, step (a) comprises measuring the level of four or more tissue-derived serum glycoproteins selected from two or more of the tissue-derived serum glycoprotein sets provided in Table 1 and wherein a level of at least three of the four or more tissue-derived serum glycoproteins in the blood sample from the subject determined to have the disease that is below or above the corresponding predetermined normal level defining the disease-associated tissue-derived blood fingerprint. In yet a further embodiment, step (a) comprises measuring the level of four or more tissue-derived serum glycoproteins selected from two or more of the tissue-derived serum glycoprotein sets provided in Table 1 and wherein a level of at least four of the four or more tissue-derived serum glycoproteins in the blood sample from the subject determined to have the disease that is below or above the corresponding predetermined normal level defining the disease-associated tissue-derived blood fingerprint. In certain embodiments, step (a) comprises measuring the level of five or more tissue-derived serum glycoproteins selected from two or more of the tissue-derived serum glycoprotein sets provided in Table 1 and wherein a level of at least five of the five or more tissue-derived serum glycoproteins in the blood sample from the subject determined to have the disease that is below or above the corresponding predetermined normal level defining the disease-associated tissue-derived blood fingerprint.

Another aspect of the present invention provides a method for detecting perturbation of a normal biological state in a subject comprising, a) contacting a blood sample from the subject with a plurality of detection reagents wherein each detection reagent is specific for one tissue-derived serum glycoprotein; wherein the tissue-derived serum glycoproteins detected by the plurality of detection reagents are selected from any one of the tissue-derived serum glycoprotein sets provided in Table 1; b) measuring the amount of the tissue-derived serum glycoprotein detected in the blood sample by each detection reagent; and c) comparing the amount of the tissue-derived serum glycoprotein detected in the blood sample by each detection reagent to a predetermined normal amount for each respective tissue-derived serum glycoprotein; wherein a statistically significant altered level in one or more of the tissue-derived serum glycoproteins indicates a perturbation in the normal biological state.

A further aspect of the invention provides a method for detecting perturbation of a normal biological state in a subject comprising, a) contacting a blood sample from the subject with a plurality of detection reagents wherein each detection reagent is specific for one tissue-derived serum glycoprotein; wherein the tissue-derived serum glycoproteins detected by the plurality of detection reagents are selected from two or more of the tissue-derived serum glycoprotein sets provided in Table 1; b) measuring the amount of the tissue-derived serum glycoprotein detected in the blood sample by each detection reagent; and c) comparing the amount of the tissue-derived serum glycoprotein detected in the blood sample by each detection reagent to a predetermined normal amount for each respective tissue-derived serum glycoprotein; wherein a statistically significant altered level in one or more of the tissue-derived serum glycoproteins indicates a perturbation in the normal biological state.

Another aspect of the invention provides a method for detecting prostate disease in a subject comprising, a) contacting a blood sample from the subject with a plurality of detection reagents wherein each detection reagent is specific for one prostate-derived protein; wherein the prostate-derived proteins are selected from the prostate-derived serum glycoprotein set provided in Table 1; b) measuring the amount of the tissue-derived serum glycoprotein detected in the blood sample by each detection reagent; and c) comparing the amount of the tissue-derived serum glycoprotein detected in the blood sample by each detection reagent to a predetermined normal control amount for each respective tissue-derived serum glycoprotein; wherein a statistically significant altered level in one or more of the tissue-derived serum glycoproteins indicates the presence of prostate disease in the subject. In this regard, the prostate disease may be prostate cancer, prostatitis, or benign prostatic hyperplasia. In one embodiment, the plurality of detection reagents comprises at least 2, 3, 4, 5, 6, 7, 8, 9, 10, or more detection reagents.

A further aspect of the invention provides a method for monitoring a response to a therapy in a subject, comprising the steps of (a) measuring in a blood sample obtained from the subject the level of a plurality of tissue-derived serum glycoproteins, wherein the plurality of tissue-derived serum glycoproteins are selected from any one of the tissue-derived serum glycoprotein sets provided in Table 1; (b) repeating step (a) using a blood sample obtained from the subject after undergoing therapy; and (c) comparing the level of the plurality of tissue-derived serum glycoproteins detected in step (b) to the amount detected in step (a) and therefrom monitoring the response to the therapy in the patient.

Yet a further aspect of the invention provides a method for monitoring a response to a therapy in a subject, comprising the steps of (a) measuring in a blood sample obtained from the subject the level of a plurality of tissue-derived serum glycoproteins, wherein the plurality of tissue-derived serum glycoproteins are selected from two or more of the tissue-derived serum glycoprotein sets provided in Table 1; (b) repeating step (a) using a blood sample obtained from the subject after undergoing therapy; and (c)comparing the level of the plurality of tissue-derived serum glycoproteins detected in step (b) to the amount detected in step (a) and therefrom monitoring the response to the therapy in the patient.

Another aspect of the invention provides a targeting agent comprising an tissue-derived probe that specifically recognizes a sequence of any one or more of the sequences set forth in Table 1, wherein said probe has attached thereto a therapeutic agent, said therapeutic agent comprising a radioisotope or cytotoxic agent.

Another aspect of the invention provides an assay device comprising a panel of detection reagents wherein each detection reagent in the panel, with the exception of a negative and positive control, is capable of specific interaction with one of a plurality of tissue-derived serum glycoproteins present in blood, wherein the plurality of tissue-derived serum glycoproteins are derived from the same tissue and wherein the pattern of interaction between the detection reagents and the tissue-derived serum glycoproteins present in a blood sample is indicative of a biological condition.

One aspect of the present invention provides a method for diagnosing a biological condition in a subject comprising measuring the level of a plurality of tissue-derived glycoproteins in the blood of the subject, wherein the plurality of tissue-derived glycoproteins are derived from the same tissue and wherein the levels of the plurality of tissue-derived glycoproteins together provide a fingerprint for the biological condition in the subject. In certain embodiments of this method the level of the plurality of tissue-derived proteins is quantified using a method selected from the group consisting of tandem mass spectrometry, ELISA, Western blot, microfluidics/nanotechnology sensors, and capture assays mediated by aptamers or other types of capture agents. In another embodiment of the method, the plurality of tissue-derived glycoproteins comprises from at least 2 tissue-derived glycoproteins to 100 or more tissue-derived glycoproteins. In this regard, the plurality of tissue-derived glycoproteins may comprise about 10 or about 20 tissue-derived glycoproteins. In certain embodiments, the tissue-derived glycoproteins comprise prostate-derived proteins. In this regard, the prostate-derived proteins are selected from the group consisting of CD13, CD14, CD26, CD44, CD45, CD56, CD90, CD91, CD107a, CD107b, CD109, CD166, CD143, CD224, PSMA-1, Glutamate carboxypeptidase II, MAC-2 binding protein, metalloproteinase inhibitor 1, and tumor endothelial marker 7-related precursor. In a further embodiment, the tissue-derived glycoproteins comprise breast-derived proteins. In this regard the breast-derived proteins are selected from the group consisting of CD71, CD98, CD107b, CD155, CD224, MAC-2 binding protein, receptor protein-tyrosine kinase erbB-2, and tumor-associated calcium signal transducer 2. In certain embodiments, the biological condition comprises a cancer. The cancer may be any one or more of prostate cancer, ovarian cancer, breast cancer, liver cancer, lung cancer, pancreatic cancer, kidney cancer, or colon cancer. Other cancers known in the art are also contemplated herein. In another embodiment, the biological condition is selected from the group consisting of cardiovascular disease, metabolic disease, infectious disease, genetic disease, autoimmune disease, immune-related disease, and cancer.

Another aspect of the invention provides a method for determining the presence or absence of disease in a subject comprising, detecting a level of each of a plurality of tissue-derived glycoproteins in a blood sample from the subject, wherein the plurality of tissue-derived glycoproteins are derived from the same tissue; comparing said level of each of the plurality of tissue-derived glycoproteins in the blood sample from the subject to a level of the plurality of tissue-derived glycoproteins in a normal control sample of blood; wherein a statistically significant altered level of one or more of the plurality of tissue-derived glycoproteins in the blood is indicative of the presence or absence of disease. In one embodiment, the level of each of the plurality of tissue-derived glycoproteins is detected using a method selected from the group consisting of mass spectrometry, and an immunoassay. In a further embodiment, the level of each of the plurality of tissue-derived glycoproteins is measured (quantified) using tandem mass spectrometry. In yet another embodiment, the level of each of the plurality of tissue-derived glycoproteins is measured using ELISA. In an additional embodiment, the level of each of the plurality of tissue-derived glycoproteins is measured using an antibody array.

Another aspect of the present invention provides a method for detecting perturbation of a normal biological state comprising, contacting a blood sample with a plurality of detection reagents each specific for a tissue-derived glycoprotein in blood, wherein each tissue-derived glycoprotein is derived from the same tissue; measuring the amount of the tissue-derived glycoprotein detected in the blood sample by each detection reagent, comparing the amount of the tissue-derived glycoprotein detected in the blood sample by each detection reagent to a predetermined control amount for each tissue-derived glycoprotein; wherein a statistically significant altered level in one or more of the tissue-derived glycoproteins indicates a perturbation in the normal biological state. In one embodiment, the plurality of detection reagents comprises from at least 2 detection reagents to about 100 detection reagents. Thus, the plurality of detection reagents may be about 10, about 20, or about 30 detection reagents. In another embodiment, the tissue-derived glycoproteins comprise prostate-derived proteins or liver-derived proteins or breast-derived proteins.

A further aspect of the present invention provides a diagnostic panel for determining the presence or absence of disease in a subject comprising, a plurality of detection reagents each specific for detecting one of a plurality of tissue-derived proteins present in a blood sample; wherein the tissue-derived proteins are derived from the same tissue and wherein detection of the plurality of tissue-derived proteins with the plurality of detection reagents results in a fingerprint indicative of the presence or absence of disease in the animal. In one embodiment, the detection reagents comprise antibodies or antigen-binding fragments thereof. In a further embodiment, the antibodies are monoclonal antibodies, or antigen-binding fragments thereof. In another embodiment, the plurality of detection reagents comprises from at least 2 detection reagents to about 100 detection reagents. In certain embodiments, the plurality of detection reagents comprises about 5 detection reagents, about 10 detection reagents, or about 20 detection reagents. In another embodiment, the tissue-derived proteins comprise prostate-derived proteins. In another embodiment, the tissue-derived proteins comprise liver-derived proteins, or breast-derived proteins. In a further embodiment, the disease comprises a cancer. In this regard, the cancer may be any one or more of prostate cancer, hematological cancer, breast cancer, liver cancer, and bladder cancer. In another embodiment, the disease is selected from the group consisting of cardiovascular disease, metabolic disease, infectious disease, genetic disease, autoimmune disease, immune-related disease, and cancer.

Another aspect of the present invention provides an assay device comprising a panel of detection reagents wherein each detection reagent in the panel, with the exception of a negative and positive control, is capable of specific interaction with one of a plurality of tissue-derived glycoproteins present in blood, wherein the plurality of tissue-derived glycoproteins are derived from the same tissue and wherein the pattern of interaction between the detection reagents and the tissue-derived glycoproteins present in a blood sample is indicative of a biological condition.

### BRIEF DESCRIPTION OF THE DRAWING(S)

FIG. 1. Schematic diagram of detection of N-linked glycopeptides from tissues/cells in plasma. 1. Protein extraction. Proteins were extracted from cells using homogenization and differential centrifugation (Han D K, Eng J, Zhou H, Aebersold R. (2001) Quantitative profiling of differentiation-induced microsomal proteins using isotope-coded affinity tags and mass spectrometry. Nat Biotechnol 19: 946-951) or from solid tissues using collagenase digestion of tissues (Liu A Y, Zhang H, Sorensen C M, Diamond D L. (2005) Analysis of prostate cancer by proteomics using tissue specimens. J Urol 173: 73-78). 2) Glycopeptide capture. Proteins from tissues/cells and plasma were processed by recently described solid-phase extraction of glycopeptides (SPEG) (Zhang H, Li X J, Martin D B, Aebersold R. (2003) Identification and quantification of N-linked glycoproteins using hydrazide chemistry, stable isotope labeling and mass spectrometry. Nat Biotechnol 21: 660-666). Peptides that contained N-linked carbohydrates in the native protein are isolated in their de-glycosylated form. 3) Peptide identification. Isolated peptides were analyzed to generate an identified peptide patterns from LC-MS/MS analysis and SEQUEST search (Eng J, McCormack A L, Yates J R, 3rd. (1994) An approach to correlate tandem mass spectral data of peptides with amino acid sequences in a protein database. J. Am. Soc. Mass Spectrom. 5: 976-989). 4) Peptide comparison. Peptides obtained from different samples were compared and peptides identified from both tissues/cells and plasma were determined.

FIG. 2. Comparison of N-linked glycosites identified from cell/tissue and plasma. The total number of N-linked glycosites and tissue-specific N-linked glycosites are compared with the N-linked glycosites identified from plasma. Peptide identification was defined as scoring ≧0.9 with PeptideProphet (Keller A, Nesvizhskii A I, Kolker E, Aebersold R. (2002) Empirical statistical model to estimate the accuracy of peptide identifications made by MS/MS and database search. Anal Chem 74: 5383-5392). An identified N-linked glycosite was defined as cell/tissue specific if it was only detected in one cell/tissue type in this study. The number of N-linked glycosites identified from the specific cell/tissue type that are common to a given cell/tissue and plasma are listed in small circles representing the cell/tissue (275, 64, 116, 307, 200, 329, 123, and 309).

FIG. 3. Tissue-derived N-linked glycosite identifications are also common to multiple tissue-types. Shown in this overlap are only the N-linked glycosites identified in prostate, bladder, or liver metastasis of prostate cancer that were also identified in plasma.

FIG. 4. Tissue/cell-derived proteins in blood. Selected proteins were identified in both tissue/cell and plasma using glycopeptide capture and MS/MS for lymphocyte cells (lym), prostate tissue (prst), bladder (blad), breast cancer cells (brst), liver metastasis (liv). Protein expression patterns as determined by immunohistochemistry (IHC) are also shown (proteins whose expression patterns not tested by IHC are marked with brick-like hatching). A full list of identified proteins is shown in Table 1.

FIG. 5: A schematic flow chart of a test for peptide antigen using quantitative immobilization of antibody.

FIG. 6: The known normal plasma concentration distribution for cell/tissue and plasma-derived N-linked glycoproteins. The histograms for those proteins identified from both cell/tissue and plasma or from cell/tissue only and that had also recently been shown to be candidate disease markers with known concentrations in normal plasma (Anderson L. (2005) Candidate-based proteomics in the search for biomarkers of cardiovascular disease. J Physiol 563: 23-60; Anderson L, Polanski M. (2006) A list of candidate cancer biomarkers for targeted proteomics. Biomarker Insights In press) (also see Table 1) are displayed. For convenience, published protein concentrations were binned across sequential plasma concentration ranges each spanning one order of magnitude and were plotted on a log scale.

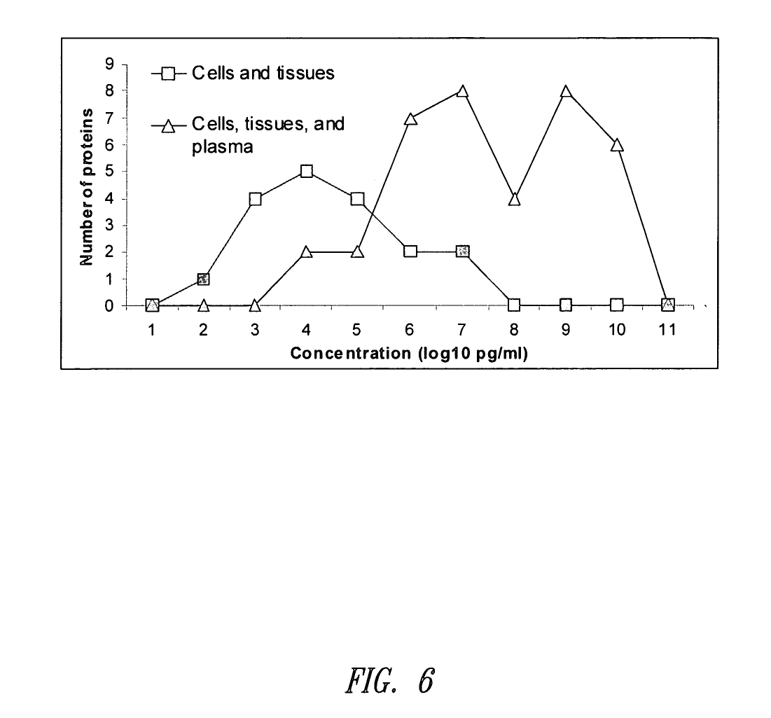

Table 1: See Example 1. Identified peptide sequences were first assigned to proteins in the IPI database (version 2.28). Assigned proteins were then mapped to RNA sequences in the RefSeq database (NCBI build number 36) using connections stored in the IPI database and in the EntrezGene database (modified on Sep. 18, 2006).

### DETAILED DESCRIPTION OF THE INVENTION

Biomarker discovery is the detection and identification of proteins in plasma that individually, or in combination, represent the health status of a specific tissue or cell-type. Such proteins released from diseased tissues or cells in relatively small amounts will be diluted significantly upon entering the blood stream relative to their levels if analyzing the tissue or cells from which they originated. Therefore, many disease-specific biomarkers are most likely to be present in plasma at a lower abundance compared with constitutive plasma proteins.

In the search for a method that has the potential to detect such tissue-derived proteins in plasma, we developed a method for high throughput analyses of glycoproteins (8). This approach is based on the idea that most cell surface and secreted proteins from tissues are glycosylated, and that disease-associated glycoproteins, either secreted by cells or shed from their surfaces, are more likely to enter into the blood stream. This explains why most currently known clinical biomarkers for blood test are also known to be glycosylated (7). To discover additional biomarkers and develop blood tests for diseases, it is critical to detect those proteins in blood that have been shown to express in disease tissues or to change their abundance in disease tissues compared to normal tissues using either genomic or proteomic approaches. Differential expression analyses have shown that many of the genes up-regulated in disease tissues represent surface or secreted proteins, and these extracellular proteins are either known to be glycosylated or likely to be glycosylated (9, 10). Thus the profiling of glycoproteins from specific tissues or cells, and comparing them to glycoproteins identified from plasma is likely to allow for the identification of tissue- and disease-specific proteins in blood.

Thus, the present invention pre-defines tissue-derived serum glycoprotein sets specifically identified and quantified for each of multiple human tissue types. These tissue-derived proteins identified from human tissues may, in whole or in part, be used as markers or identifiers for health and disease. The levels of these tissue-derived serum glycoproteins in blood from diseased individuals may be distinguished from the levels of these tissue-derived serum glycoproteins in the blood of healthy individuals. By identifying tissue-derived serum glycoprotein markers and measuring the level of these glycoproteins in normal blood, the status of health or disease may be monitored through the correlation of the levels of glycoproteins in the tissue-derived serum glycoprotein fingerprint at the earliest stages of disease and lead to early diagnosis and treatment.

Thus, the present invention provides tissue-derived serum glycoproteins that serve as markers to measure changes in the status of a tissue or tissues to measure health and diagnose disease.

The inventive markers are used as a library of biological indicators to identify tissue-derived glycoproteins that are secreted, leaked, excreted or shed into blood in a human or mammal. Such markers can be used individually or collectively. For example a single marker for an organ or tissue could be used to monitor that organ or tissue. However, adding additional markers detected in that tissue and also detected in plasma to the assay will improve the diagnostic power as well as the sensitivity of the assay. Further, one of skill in the art can readily appreciate that probes to such markers, be they nucleic acid probes, nanoparticles, or polypeptides (e.g., antibodies) can comprise a kit, lateral flow test kit or an array and can include a few probes to several tissues or several to one tissue. For example, in one kit or assay device a whole body health assay may be used wherein several markers are tracked for every tissue and when one or more tissues demonstrates a deviation from normal a more rigorous test is performed with many more markers for that tissue. Likewise, entire tissue set assays may be devised. In such an example a cardiovascular assay may be employed wherein tissue-specific markers from heart and lung are the basis of the assay kit.

One of skill in the art can readily appreciate that the application of these tissue-derived serum marker sets are virtually limitless. From using as diagositic and prognostic indicators, to use in following drug treatment or in drug discovery to determine what proteins and genes are affected. Further, such markers can easily be used in combination with antibodies for other ligands for drug targeting or imaging via MRI or PET or by other means. In such examples, a prostate-derived serum glycoprotein marker could form the basis for targeted cancer therapy or possible imaging/therapy of metastatic cancer derived from prostate. The comparison of the normal levels of tissue-derived serum glycoproteins to the levels of these glycoproteins found in a sample of patient blood or bodily fluid or other biological sample, such as a biopsy can be used to define normal health, detect the early stages of disease, monitor treatment, prognosticate disease, measure drug responses, titrate administered drug doses, evaluate efficacy, stratify patients according to disease type (e.g., prostate cancer may well have four or more major types) and define therapeutic targets when therapeutic intervention is most effective.

The present invention provides for the identification of N-linked glycopeptides and glycoproteins from tissues and cells, as well as the detection of many of these proteins in plasma via glycopeptide capture and liquid chromatography tandem mass spectrometry (LC-MS/MS) (8). Thus, the methods, compositions, and panels of the present invention can be used to detect tissue-derived and perturbed glycoproteins and/or glycosites in plasma and perturbations in the expression of these glycoproteins/glycosites in plasma. As discussed further herein, in certain embodiments of the invention, it may be desirable to detect one or more glycosites as opposed to the glycoproteins that contain them. In this way, the concentration limit of detection can be significantly improved due to the reduction in sample complexity. Thus, anywhere that detection or quantitation of a glycoprotein is described herein, detection or quantitation of a glycosite may be substituted therefor and may be more desirable in certain embodiments. Accordingly, the present invention is useful for the diagnosis and monitoring of diseases and treatments.

It should be noted that the number of N-glycosites in the human proteome is finite and quite well known to the skilled artisan. This means that all the glycosites can be identified and, therefore, the comparison between the patterns of expression of glycosites in various tissues becomes more meaningful. This is because in all other proteomic methods, the proteome is under-sampled and it is impossible to know whether a protein is not present in a given sample or is simply not being detected. However, if all the glycosites are known, then it is possible to distinguish between a peptide not being present and a protein not being detected.

The term “blood” refers to whole blood, plasma or serum obtained from a mammal.

In the practice of the invention, an “individual” or “subject” refers to vertebrates, particularly members of a mammalian species, and includes, but is not limited to, primates, including human and non-human primates, domestic animals, and sports animals.

“Component” or “member” of a set refers to an individual constituent protein, peptide, nucleotide or polynucleotide of a tissue-specific set.

As used herein, the term “plasma” refers to plasma or serum.

As used herein, the term “serum” refers to serum or plasma.

As used herein, the term “polypeptide”” is used in its conventional meaning, i.e., as a sequence of amino acids. The polypeptides are not limited to a specific length of the product; thus, peptides, oligopeptides, and proteins are included within the definition of polypeptide, and such terms may be used interchangeably herein unless specifically indicated otherwise. A polypeptide can also be modified by naturally occurring modifications such as post-translational modifications, including phosphorylation, fatty acylation, prenylation, sulfation, hydroxylation, acetylation, addition of carbohydrate, addition of prosthetic groups or cofactors, formation of disulfide bonds, proteolysis, assembly into macromolecular complexes, and the like. A “peptide fragment” is a peptide of two or more amino acids, generally derived from a larger polypeptide.

As used herein, a “glycopolypeptide”, “glycoprotein”, or “glycopeptide” refers to a polypeptide that contains a covalently bound carbohydrate group. The carbohydrate can be a monosaccharide, oligosaccharide or polysaccharide. Proteoglycans are included within the meaning of “glycopolypeptide.” A glycopolypeptide can additionally contain other post-translational modifications. A “glycopeptide” refers to a peptide that contains covalently bound carbohydrate. A “glycopeptide fragment” refers to a peptide fragment resulting from enzymatic or chemical cleavage of a larger polypeptide in which the peptide fragment retains covalently bound carbohydrate. It is understood that a glycopeptide fragment or peptide fragment refers to the peptides that result from a particular cleavage reaction, regardless of whether the resulting peptide was present before or after the cleavage reaction. Thus, a peptide that does not contain a cleavage site will be present after the cleavage reaction and is considered to be a peptide fragment resulting from that particular cleavage reaction. For example, if bound glycopeptides are cleaved, the resulting cleavage products retaining bound carbohydrate are considered to be glycopeptide fragments. The glycosylated fragments can remain bound to the solid support, and such bound glycopeptide fragments are considered to include those fragments that were not cleaved due to the absence of a cleavage site.

As disclosed herein, a glycopolypeptide, glycopeptide, or glycoprotein can be processed such that the carbohydrate is removed from the parent glycopolypeptide. It is understood that such an originally glycosylated polypeptide is still referred to herein as a glycopolypeptide, glycopeptide, or glycoprotein even if the carbohydrate is removed enzymatically and/or chemically. Thus, a glycopolypeptide or glycopeptide can refer to a glycosylated or de-glycosylated form of a polypeptide. A glycopolypeptide, glycopeptide, or glycoprotein from which the carbohydrate is removed is referred to as the de-glycosylated form of a polypeptide whereas a glycopolypeptide or glycopeptide which retains its carbohydrate is referred to as the glycosylated form of a polypeptide

As used herein, “tissue-derived serum glycoprotein set” refers to a set of glycoproteins detected in serum that are also detected in one or more tissues. A tissue-derived serum glycoprotein set may include glycoproteins detected in serum that are expressed (and detected) only in a single tissue (e.g., a prostate-specific glycoprotein) and may also include glycoproteins that are expressed in multiple tissues (see Table 1). Illustrative tissue-derived serum glycoprotein sets are set forth in Table 1. For example, the prostate-derived serum glycoprotein set is comprised of the glycoproteins listed in Table 1 that are detected in prostate (as indicated by the table entries that contain the number 1) and also detected in plasma. Similarly, the bladder tissue-derived serum glycoprotein set is comprised of the glycoproteins detected in bladder and also detected in plasma. Note that some glycoproteins may be present in more than one tissue-derived serum glycoprotein set (e.g., Swiss Prot No. P07711 Cathepsin L precursor is in the prostate, bladder, liver and breast tissue-derived serum glycoprotein sets).

As used herein, “N-glycosite” or “glycosite” is defined as a peptide that is N-glycosylated in the intact protein.

As used herein, “tissue-derived serum glycosite set” refers to a set of glycosites (e.g. glycopeptides) identified from serum that are also identified in one or more tissues. A tissue-derived serum glycosite set may include glycosites identified in serum that are detected only in a single tissue (e.g., a prostate-specific glycosite) and may also include glycosites that are identified in multiple tissues (see Table 1). Illustrative tissue-derived serum glycosite sets are set forth in Table 1. For example, the prostate-derived serum glycosite set is comprised of the glycosites listed in Table 1 that are identified in prostate (as indicated by those cells that contain the number 1) and also detected in plasma. Similarly, the bladder tissue-derived serum glycosite set is comprised of the glycosites identified from bladder and also from plasma. Note that some glycosites may be present in more than one tissue-derived serum glycosite set (e.g., Swiss Prot No. P07711 Cathepsin L precursor was identified in prostate, bladder, liver and breast tissues as well as in serum). It should also be noted that a given glycosite may map to multiple glycoproteins. In other words, multiple glycoproteins contain the same glycosite. In certain embodiments of the invention, it may be desirable to detect one or more glycosites as opposed to the glycoproteins that contain them. In this way, the concentration limit of detection is significantly improved due to the reduction in sample complexity. Thus, anywhere that detection or quantitation of a glycoprotein is described herein, detection or quantitation of a glycosite may be substituted therefor and may be more desirable in certain embodiments.

The methods described herein such as those disclosed in Example 1, describe the detection of glycoproteins. It should be noted that these methods in fact, detect the N-glycosite, defined as a peptide that is N-glycosylated in the intact protein. (These methods can be extended to detect O-linked proteins). From the identified N-glycosites the presence of a glycoprotein is inferred.

As used herein, a “normal tissue-derived serum glycoprotein fingerprint” is a data set comprising the determined levels in blood from normal, healthy individuals of one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, thirty, thirty-one, thirty-two, thirty-three, thirty-four, thirty-five, thirty-six, thirty-seven, thirty-eight, thirty-nine, forty, forty-one, forty-two, forty-three, forty-four, forty-five, forty-six, forty-seven, forty-eight, forty-nine, fifty, sixty, seventy, eighty, ninety, one-hundred or more components of a tissue-derived serum glycoprotein set of one tissue, but could comprise multiples thereof if more than one tissue is analyzed. The normal levels in the blood for each component included in a fingerprint are determined by measuring the level of protein in the blood using any of a variety of techniques known in the art and described herein, in a sufficient number of blood samples from normal, healthy individuals to determine the standard deviation (SD) with statistically meaningful accuracy. Thus, as would be recognized by one of skill in the art, a determined normal level is defined by averaging the level of protein measured in a statistically large number of blood samples from normal, healthy individuals and thereby defining a statistical range of normal. A normal tissue-derived serum glycoprotein fingerprint comprises the determined levels in normal, healthy blood of N members of a tissue-derived serum glycoprotein set wherein N is 1, 2, 3, 4, 5, 6, 7, 8, 9, 10, 11, 12, 13, 14, 15, or more members up to the total number of members in a given tissue-derived serum glycoprotein set per tissue being profiled. In certain embodiments, a normal tissue-derived serum glycoprotein fingerprint comprises the determined levels in normal, healthy blood of at least two components of a tissue-derived serum glycoprotein set. In other embodiments, a normal tissue-derived serum glycoprotein fingerprint comprises the determined levels in normal, healthy blood of at least 3, 4, 5, 6, 7, 8, 9, 10, 11, 12, 13, 14, 15, 16, 17, 18, 19, or 20 components of a tissue-derived serum glycoprotein set. In yet further embodiments, a normal tissue-derived serum glycoprotein fingerprint comprises the presence or absence of cell or tissue-derived proteins or transcripts and may or may not rely on absolute levels of said components per se. In specific embodiments, merely a change over a baseline measurement for a particular individual glycoprotein may be used. In such an embodiment, levels or mere presence or absence of proteins or transcripts from blood, body fluid or tissue may be measured at one time point and then compared to a subsequent measurement, hours, days, months or years later. Accordingly, normal changes per individual can be zeroed out and only those proteins or transcripts that change over time are focused on.

As used herein, a “predetermined normal level” is an average of the levels of a given component measured in a statistically large number of blood samples from normal, healthy individuals. Thus, a predetermined normal level is a statistical range of normal and is also referred to herein as “predetermined normal range”. The normal levels or range of levels in the blood for each component are determined by measuring the level of protein in the blood using any of a variety of techniques known in the art and described herein in a sufficient number of blood samples from normal, healthy individuals to determine the standard deviation (SD) with statistically meaningful accuracy. In one embodiment it may be useful to determine average levels for individuals falling into different age groups (e.g. 1-2, 3-5, 6-8, 9-12 and so forth if, indeed, these levels change with age). In another embodiment, one may also want to determine the levels at certain times of the day, at certain times from having eaten a meal, etc. One may also determine how common physiological stimuli affect the tissue-derived serum glycoprotein fingerprints.

As used herein a “disease-associated tissue-derived serum glycoprotein fingerprint” is a data set comprising the determined level in a blood sample from an individual afflicted with a disease of one or more components of a normal tissue-derived serum glycoprotein set that demonstrates a statistically significant change as compared to the determined normal level (e.g., wherein the level in the disease sample is above or below a predetermined normal range). The data set is compiled from samples from individuals who are determined to have a particular disease using established medical diagnostics for the particular disease. The blood (serum) level of each protein member of a normal tissue-derived serum glycoprotein set as measured in the blood of the diseased sample is compared to the corresponding determined normal level. A statistically significant variation from the determined normal level for one or more members of the normal serum tissue-derived protein set provides diagnostically useful information (disease-associated fingerprint) for that disease. Thus, note that it may be determined for a particular disease or disease state that the level of only a few members of the normal tissue-derived serum protein set change relative to the normal levels. Thus, a disease-associated tissue-derived serum glycoprotein fingerprint may comprise the determined levels in the blood of only a subset of the components of a normal tissue-derived serum glycoprotein set for a given tissue and a particular disease. Thus, a disease-associated tissue-derived blood fingerprint comprises the determined levels in blood (or as noted herein any bodily fluid or tissue sample, however in most embodiments samples from blood are compared with a normal from blood and so on) of N members of a tissue-derived serum glycoprotein set wherein N is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 110 or more or any integer value therebetween, or more members up to the total number of members in a given tissue-derived serum glycoprotein set tissue-derived serum glycoprotein set. In this regard, in certain embodiments, a disease-associated tissue-derived blood fingerprint comprises the determined levels of one or more components of a normal tissue-derived serum glycoprotein set. In one embodiment, a disease-associated tissue-derived blood fingerprint comprises the determined levels of at least two components of a normal tissue-derived serum glycoprotein set. In other embodiments, a disease-associated tissue-derived blood fingerprint comprises the determined levels of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 110 or more or any integer value therebetween components of a normal tissue-derived serum glycoprotein set.

Because the disease-perturbed networks in a tissue may initiate the expression of one or more proteins whose synthesis it does not ordinarily control, it should be noted that, in certain embodiments, a disease-associated tissue-derived blood fingerprint will comprise the determined level of one or more components that are detected in tissue but that are not normally detected in serum (see Table 1). As discussed further herein (see Example 1), Prostate Specific Antigen (PSA) is detected in prostate tissue using the methods described herein, but is not normally detected in serum. However, as would be appreciated by the skilled artisan, this protein is detectable in serum in individuals with prostate cancer. Thus, in certain embodiments, the disease-associated tissue-derived blood fingerprint will include the measured levels of one or more glycoproteins detected in tissue that may not have been detected in normal serum. Illustrative glycoproteins include those tissue-derived glycoproteins described in Table 1. Thus, in this regard, a disease-associated tissue-derived blood fingerprint may comprise the determined level of one or more components of a normal tissue-derived serum glycoprotein set or may comprise a glycoprotein or set of glycoproteins not detected in a normal tissue-derived serum glycoprotein set. Further, in certain embodiments, a disease-associated “tissue-derived” blood fingerprint comprises the determined levels of one or more components of one, two, three, four, five, six, seven, eight, nine, ten, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 110 or any integer value therebetween or more normal tissue-derived serum glycoprotein sets. Further, in additional embodiments, the at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 110 or more or any integer value therebetween components of multiple sets could be combined for analysis of multiple organs, tissues, systems, or cells. Thus, in this regard, a disease-associated tissue-derived blood fingerprint may comprise the determined levels of one or more components from 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 110 or any integer value therebetween or more normal tissue-derived serum glycoprotein sets.

Note that, since multiple glycoproteins may contain the same glycosite, the level of multiple proteins containing a given glycosite can be quantified using a single detection reagent that binds to the given glycosite. Thus, as would be understood by the skilled artisan, the present invention also contemplates measuring the level of one or more glycoproteins by direct detection of a glycosite. As would be appreciated by the skilled artisan, detection reagents that bind to glycosites can be generated using any of a variety of methods known in the art and described herein. For example, glycosites can be detected and quantified as described in Example 1 or using antibodies as would be understood by the skilled artisan using methods known in the art and described herein.

The term “test compound” refers in general to a compound to which a test cell is exposed, about which one desires to collect data. Typical test compounds will be small organic molecules, typically prospective pharmaceutical lead compounds, but can include proteins (e.g., antibodies), peptides, polynucleotides, heterologous genes (in expression systems), plasmids, polynucleotide analogs, peptide analogs, lipids, carbohydrates, viruses, phage, parasites, and the like.

The term “biological activity” as used herein refers to the ability of a test compound to alter the expression of one or more genes or proteins.

The term “test cell” refers to a biological system or a model of a biological system capable of reacting to the presence of a test compound, typically a eukaryotic cell or tissue sample, or a prokaryotic organism.

The term “gene expression profile” refers to a representation of the expression level of a plurality of genes in response to a selected expression condition (for example, incubation in the presence of a standard compound or test compound). Gene expression profiles can be expressed in terms of an absolute quantity of mRNA transcribed for each gene, as a ratio of mRNA transcribed in a test cell as compared with a control cell, and the like or the mere presence or absence of a protein an RNA transcript or more generally gene expression. As used herein, a “standard” gene expression profile refers to a profile already present in the primary database (for example, a profile obtained by incubation of a test cell with a standard compound, such as a drug of known activity), while a “test” gene expression profile refers to a profile generated under the conditions being investigated. The term “modulated” refers to an alteration in the expression level (induction or repression) to a measurable or detectable degree, as compared to a pre-established standard (for example, the expression level of a selected tissue or cell type at a selected phase under selected conditions).

“Similar”, as used herein, refers to a degree of difference between two quantities that is within a preselected threshold. The similarity of two profiles can be defined in a number of different ways, for example in terms of the number of identical genes affected, the degree to which each gene is affected, and the like. Several different measures of similarity, or methods of scoring similarity, can be made available to the user: for example, one measure of similarity considers each gene that is induced (or repressed) past a threshold level, and increases the score for each gene in which both profiles indicate induction (or repression) of that gene.

As used herein, the term “target specific” is intended to mean an agent that binds to a target analyte selectively. This agent will bind with preferential affinity toward the target while showing little to no detectable cross-reactivity toward other molecules. For example, when the target is a nucleic acid, a target specific sequence is one that is complementary to the sequence of the target and able to hybridize to the target sequence with little to no detectable cross-reactivity with other nucleic acid molecules. A nucleic acid target could also be bound in a target specific manner by a protein, for example by the DNA binding domain of a transcription factor. If the target is a protein or peptide it can be bound specifically by a nucleic acid aptamer, or another protein or peptide, or by an antibody or antibody fragment which are sub-classes of proteins.

As used herein, the term “genedigit” is intended to mean a region of pre-determined nucleotide or amino acid sequence that serves as an attachment point for a label. The genedigit can have any structure including, for example, a single unique sequence or a sequence containing repeated core elements. Each genedigit has a unique sequence which differentiates it from other genedigits. An “anti-genedigit” is a nucleotide or amino acid sequence or structure that binds specifically to the gene digit. For example, if the genedigit is a nucleic acid, the anti-genedigit can be a nucleic acid sequence that is complementary to the genedigit sequence. If the genedigit is a nucleic acid that contains repeated core elements then the anti-genedigit can be a series of repeat sequences that are complementary to the repeat sequences in the genedigit. An anti-genedigit can contain the same number, or a lesser number, of repeat sequences compared to the genedigit as long as the anti-genedigit is able to specifically bind to the genedigit.

As used herein, the term “specifier” is intended to mean the linkage of one or more genedigits to a target specific sequence. The genedigits can be directly linked or can be attached using an intervening or adapting sequence. A specifier can contain a target specific sequence which will allow it to bind to a target analyate. An “anti-specifier” has a complementary sequence to all or part of the specifier such that it specifically binds to the specifier.

As used herein, the term “label” is intended to mean a molecule or molecules that render an analyte detectable by an analytical method. Appropriate labels depends on the particular assay format and are well known by those skilled in the art. For example, a label specific for a nucleic acid molecule can be a complementary nucleic acid molecule attached to a label monomer or measurable moiety, such as a radioisotope, fluorochrome, dye, enzyme, nanoparticle, chemiluminescent marker, biotin, or other moiety known in the art that is measurable by analytical methods. In addition, a label can include any combination of label monomers.

As used herein, “unique” when used in reference to a label is intended to mean a label that has a detectable signal that distinguishes it from other labels in the same mixture. Therefore, a unique label is a relative term since it is dependent upon the other labels that are present in the mixture and the sensitivity of the detection equipment that is used. In the case of a fluorescent label, a unique label is a label that has spectral properties that significantly differentiate it from other fluorescent labels in the same mixture. For example, a fluorescein label can be a unique label if it is included in a mixture that contains a rhodamine label since these fluorescent labels emit light at distinct, essentially non-overlapping wavelengths. However, if another fluorescent label was added to the mixture that emitted light at the same or very similar wavelength to fluorescein, for example the Oregon Green fluorophore, then the fluorescein would no longer be a unique label since Oregon Green and fluorescein could not be distinguished from each other. A unique label is also relative to the sensitivity of the detection equipment used. For example, a FACS machine can be used to detect the emission peaks from different fluorophore-containing labels. If a particular set of labels have emission peaks that are separated by, for example, 2 nm these labels would not be unique if detected on a FACS machine that can distinguish peaks that are separated by 10 nm or greater, but these labels would be unique if detected on a FACS machine that can distinguish peaks separated by 1 nm or greater.

As used herein, the term “signal” is intended to mean a detectable, physical quantity or impulse by which information on the presence of an analyte can be determined. Therefore, a signal is the read-out or measurable component of detection. A signal includes, for example, fluorescence, luminescence, calorimetric, density, image, sound, voltage, current, magnetic field and mass. Therefore, the term “unit signal” as used herein is intended to mean a specified quantity of a signal in terms of which the magnitudes of other quantities of signals of the same kind can be stated. Detection equipment can count signals of the same type and display the amount of signal in terms of a common unit. For example, a nucleic acid can be radioactively labeled at one nucleotide position and another nucleic acid can be radioactively labeled at three nucleotide positions. The radioactive particles emitted by each nucleic acid can be detected and quantified, for example in a scintillation counter, and displayed as the number of counts per minute (cpm). The nucleic acid labeled at three positions will emit about three times the number of radioactive particles as the nucleic acid labeled at one position and hence about three times the number of cpms will be recorded.

The term “polynucleotide” refers to a polymeric form of nucleotides of any length, including deoxyribonucleotides or ribonucleotides, which can comprise analogs thereof.

As used herein, “purified” refers to a specific protein, polypeptide, or peptide composition that has been subjected to fractionation to remove various other proteins, polypeptides, or peptides, and which composition substantially retains its activity, as may be assessed, for example, by any of a variety of protein assays known to the skilled artisan for the specific or desired protein, polypeptide or peptide.

The terms “polypeptide”, “peptide” and “protein” are used interchangeably herein to refer to polymers of amino acids of any length. The terms also encompass an amino acid polymer that has been modified; for example, by disulfide bond formation, glycosylation, lipidation, or conjugation with a labeling component.

Methods for Identifying Tissue- and Plasma-Derived Proteins

The present invention provides methods for identifying tissue-derived proteins in blood. Any tissue of a mammalian body is contemplated herein. Illustrative tissues include, but are not limited to tissues from heart, kidney, ureter, bladder, urethra, liver, prostate, heart, blood vessels, bone marrow, skeletal muscle, smooth muscle, brain (amygdala, caudatenucleus, cerebellum, corpus callosum, fetal, hypothalamus, thalamus), spinal cord, peripheral nerves, retina, nose, trachea, lungs, mouth, salivary gland, esophagus, stomach, small intestines, large intestines, hypothalamus, pituitary, thyroid, pancreas, adrenal glands, ovaries, oviducts, uterus, placenta, vagina, mammary glands, testes, seminal vesicles, penis, lymph nodes, PBMC, thymus, and spleen, and any cells that make up such tissues. In certain embodiments, in each of these tissues, glycoproteins are obtained for the cell types in which a disease of interest arises. For example, in the prostate there are two dominant types of cells—epithelial cells and stromal cells. About 98% of prostate cancers arise in epithelial cells. As such, in certain embodiments, tissue-derived means the glycoproteins derived from in particular cell types of the tissue of interest (e.g., prostate epithelial cells). In this regard, any cell type that makes up any of the tissues described herein is contemplated herein. Illustrative cell types include, but are not limited to, epithelial cells, stromal cells, endothelial cells, endodermal cells, ectodermal cells, mesodermal cells, lymphocytes (e.g., B cells and T cells including CD4+ T helper 1 or T helper 2 type cells, CD8+ cytotoxic T cells), erythrocytes, keratinocytes, and fibroblasts. Particular cell types within tissues may be obtained by histological dissection, by the use of specific cell lines (e.g., prostate epithelial cell lines), by cell sorting or by a variety of other techniques known in the art.

In one embodiment, glycoproteins are isolated from any of a variety of tissue samples or plasma using methods as described in US Patent Application No. 20040023306. In particular, the methods of the invention can be used to purify glycosylated proteins or peptides and identify and quantify the glycosylation sites (“glycosites”). Because the methods of the invention are directed to isolating glycopolypeptides, the methods also reduce the complexity of analysis since many proteins and fragments of glycoproteins do not contain carbohydrate. This can simplify the analysis of complex biological samples such as serum. The methods of the invention are advantageous for the determination of protein glycosylation in glycome studies and can be used to isolate and identify glycoproteins from cell membrane or body fluids to determine specific glycoprotein changes related to certain disease states or cancer. The methods of the invention can be used for detecting quantitative changes in protein samples containing glycoproteins and to detect their extent of glycosylation. The methods of the invention are applicable for the identification and/or characterization of diagnostic biomarkers, immunotherapy, or other diagnositic or therapeutic applications. The methods of the invention can also be used to evaluate the effectiveness of drugs during drug development, optimal dosing, toxicology, drug targeting, and related therapeutic applications.

In one embodiment, the cis-diol groups of carbohydrates in glycoproteins can be oxidized by periodate oxidation to give a di-aldehyde, which is reactive to a hydrazide gel with an agarose (or other suitable solid matrix) support to form covalent hydrazone bonds. The immobilized glycoproteins are subjected to protease digestion followed by extensive washing to remove the non-glycosylated peptides. The immobilized glycopeptides are released from beads by chemicals or glycosidases. The isolated peptides are analyzed by mass spectrometry (MS), and the glycopeptide sequence and corresponding proteins are identified by MS/MS combined with a database search. The glycopeptides can also be isotopically labeled, for example, at the amino or carboxyl termini to allow the quantities of glycopeptides from different biological samples to be compared.

The methods of the invention are based on selectively isolating glycosylated peptides, or peptides that were glycosylated in the original protein sample, from a complex sample. The sample consists of peptide fragments of proteins generated, for example, by enzymatic digestion or chemical cleavage. A stable isotope tag is introduced into the isolated peptide fragments to facilitate mass spectrometric analysis and accurate quantification of the peptide fragments.

The invention provides a method for identifying and quantifying glycopolypeptides in a sample. The method can include the steps of derivatizing glycopolypeptides in a polypeptide sample, for example, by oxidation; immobilizing the derivatized glycopolypeptides to a solid support; cleaving the immobilized glycopolypeptides, thereby releasing non-glycosylated peptide fragments and retaining immobilized glycopeptide fragments; optionally labeling the immobilized glycopeptide fragments with an isotope tag; releasing the glycopeptide fragments from the solid support, thereby generating released glycopeptide fragments; analyzing the released glycopeptide fragments or their de-glycosylated counterparts using mass spectrometry; and quantifying the amount of the identified glycopeptide fragment. The released glycopolypeptides can be released with the carbohydrate still attached (the glycosylated form) or with the carbohydrate removed (the de-glycosylated form).

A sample containing glycopolypeptides is chemically modified so that carbohydrates of the glycopolypeptides in the sample can be selectively bound to a solid support. For example, the glycopolypeptides can be bound covalently to a solid support by chemically modifying the carbohydrate so that the carbohydrate can covalently bind to a reactive group on a solid support. In certain embodiments, the carbohydrates of the sample glycopolypeptides are oxidized. The carbohydrate can be oxidized, for example, to aldehydes. The oxidized moiety, such as an aldehyde moiety, of the glycopolypeptides can react with a solid support containing hydrazide or amine moieties, allowing covalent attachment of glycosylated polypeptides to a solid support via hydrazine chemistry. The sample glycopolypeptides are immobilized through the chemically modified carbohydrate, for example, the aldehyde, allowing the removal of non-glycosylated sample proteins by washing of the solid support. If desired, the immobilized glycopolypeptides can be denatured and/or reduced. The immobilized glycopolypeptides are cleaved into fragments using either protease or chemical cleavage. Cleavage results in the release of peptide fragments that do not contain carbohydrate and are therefore not immobilized. These released non-glycosylated peptide fragments optionally can be further characterized, if desired.

Glycopeptides can be glycosylated peptides of any length. In this regard, the glycopeptides can be anywhere from 1-100, 200, 300, 400, 500, 1000 amino acids in length or longer. In certain embodiments, the glycopeptides are 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, or more amino acids long. They can be the molecules isolated from the natural source or generated by processing, e.g protoeolysis of such polypeptides. Thus, glycocapture can be on intact proteins or on peptides.

Following cleavage, glycosylated peptide fragments (glycopeptide fragments) remain bound to the solid support. To facilitate quantitative mass spectrometry (MS) analysis, immobilized glycopeptide fragments can be isotopically labeled. If it is desired to characterize most or all of the immobilized glycopeptide fragments, the isotope tagging reagent contains an amino or carboxyl reactive group so that the N-terminus or C-terminus of the glycopeptide fragments can be labeled. The immobilized glycopeptide fragments can be cleaved from the solid support chemically or enzymatically, for example, using glycosidases such as N-glycanase (N-glycosidase). There is no O-glycanase that is equivalent to N-glycanase. As would be understood by the skilled artisan, any of a variety of chemical reaction can be used to cleave O-linked peptides e.g beta elimination or a series of enzyme reactions.

The released glycopeptide fragments or their deglycosylated forms can be analyzed, for example, using MS.

As disclosed herein, a glycopolypeptide or glycopeptide can be processed such that the carbohydrate is removed from the parent glycopolypeptide. It is understood that such an originally glycosylated polypeptide is still referred to herein as a glycopolypeptide or glycopeptide even if the carbohydrate is removed enzymatically and/or chemically. Thus, a glycopolypeptide or glycopeptide can refer to a glycosylated or de-glycosylated form of a polypeptide. A glycopolypeptide or glycopeptide from which the carbohydrate is removed is referred to as the de-glycosylated form of a polypeptide whereas a glycopolypeptide or glycopeptide which retains its carbohydrate is referred to as the glycosylated form of a polypeptide.

As used herein, the term “sample” is intended to mean any biological fluid, cell, tissue, organ or portion thereof, that includes one or more different molecules such as nucleic acids, polypeptides, or small molecules. A sample can be a tissue section obtained by biopsy, or cells that are placed in or adapted to tissue culture. A sample can also be a biological fluid specimen such as blood, serum or plasma, cerebrospinal fluid, urine, saliva, seminal plasma, pancreatic fluid, breast milk, lung lavage, and the like. A sample can additionally be a cell extract from any species, including prokaryotic and eukaryotic cells as well as viruses. A tissue or biological fluid specimen can be further fractionated, if desired, to a fraction containing particular cell types.

As used herein, a “polypeptide sample” refers to a sample containing two or more different polypeptides. A polypeptide sample can include tens, hundreds, or even thousands or more different polypeptides. A polypeptide sample can also include non-protein molecules so long as the sample contains polypeptides. A polypeptide sample can be a whole cell or tissue extract or can be a biological fluid. Furthermore, a polypeptide sample can be fractionated using well known methods, as disclosed herein, into partially or substantially purified protein fractions.

The use of biological fluids such as a body fluid as a sample source is particularly useful in methods of the invention. Biological fluid specimens are generally readily accessible and available in relatively large quantities for clinical analysis. Biological fluids can be used to analyze diagnostic and prognostic markers for various diseases. In addition to ready accessibility, body fluid specimens do not require any prior knowledge of the specific organ or the specific site in an organ that might be affected by disease. Because body fluids, in particular blood, are in contact with numerous body organs, body fluids “pick up” molecular signatures indicating pathology due to secretion or cell lysis associated with a pathological condition. Body fluids also pick up molecular signatures that are suitable for evaluating drug dosage, drug targets and/or toxic effects, as disclosed herein.

The methods of the invention utilize the selective isolation of glycopolypeptides coupled with chemical modification to facilitate MS analysis. Proteins are glycosylated by complex enzymatic mechanisms, typically at the side chains of serine or threonine residues (O-linked) or the side chains of asparagine residues (N-linked). N-linked glycosylation sites generally fall into a sequence motif that can be described as N—X—S/T, where X can be any amino acid except proline. Glycosylation plays an important function in many biological processes (reviewed in Helenius and Aebi, Science 291:2364-2369 (2001); Rudd et al., Science 291:2370-2375 (2001)).

Protein glycosylation has long been recognized as a very common post-translational modification. As discussed above, carbohydrates are linked to serine or threonine residues (O-linked glycosylation) or to asparagine residues (N-linked glycosylation) (Varki et al. Essentials of Glycobiology Cold Spring Harbor Laboratory (1999)). Protein glycosylation, and in particular N-linked glycosylation, is prevalent in proteins destined for extracellular environments (Roth, Chem. Rev. 102:285-303 (2002)). These include proteins on the extracellular side of the plasma membrane, secreted proteins, and proteins contained in body fluids, for example, blood serum, cerebrospinal fluid, urine, breast milk, saliva, lung lavage fluid, pancreatic fluid, and the like. These also happen to be the proteins in the human body that are most easily accessible for diagnostic and therapeutic purposes.

Disclosed herein is a method for quantitative glycoprotein profiling. In one embodiment, the method is based on the conjugation of glycoproteins to a solid support using hydrazide chemistry, stable isotope labeling of glycopeptides, and the specific release of formerly N-linked glycosylated peptides via Peptide-N-Glycosidase F (PNGase F). The recovered peptides are then identified and quantified by tandem mass spectrometry (MS/MS). The method was applied to the analysis of cell surface and serum proteins, as disclosed herein.

To selectively isolate glycopolypeptides, the methods utilize chemistry and/or binding interactions that are specific for carbohydrate moieties. Selective binding of glycopolypeptides refers to the preferential binding of glycopolypeptides over non-glycosylated peptides. The methods of the invention can utilize covalent coupling of glycopolypeptides, which is particularly useful for increasing the selective isolation of glycopolypeptides by allowing stringent washing to remove non-specifically bound, non-glycosylated polypeptides.

The carbohydrate moieties of a glycopolypeptide are chemically or enzymatically modified to generate a reactive group that can be selectively bound to a solid support having a corresponding reactive group. In the embodiment, the carbohydrates of glycopolypeptides are oxidized to aldehydes. The oxidation can be performed, for example, with sodium periodate. The hydroxyl groups of a carbohydrate can also be derivatized by epoxides or oxiranes, alkyl halogen, carbonyldiimidazoles, N,N′-disuccinimidyl carbonates, N-hydroxycuccinimidyl chloroformates, and the like. The hydroxyl groups of a carbohydrate can also be oxidized by enzymes to create reactive groups such as aldehyde groups. For example, galactose oxidase oxidizes terminal galactose or N-acetyl-D-galactose residues to form C-6 aldehyde groups. These derivatized groups can be conjugated to amine- or hydrazide-containing moieties.

The oxidation of hydroxyl groups to aldehyde using sodium periodate is specific for the carbohydrate of a glycopeptide. Sodium periodate can oxidize hydroxyl groups on adjacent carbon atoms, forming an aldehyde for coupling with amine- or hydrazide-containing molecules. Sodium periodate also reacts with hydroxylamine derivatives, compounds containing a primary amine and a secondary hydroxyl group on adjacent carbon atoms. This reaction is used to create reactive aldehydes on N-terminal serine residues of peptides. A serine residue is rare at the N-terminus of a protein. The oxidation to an aldehyde using sodium periodate is therefore specific for the carbohydrate groups of a glycopolypeptide.

Once the carbohydrate of a glycopolypeptide is modified, for example, by oxidition to aldehydes, the modified carbohydrates can bind to a solid support containing hydrazide or amine moieties, such as a hydrazide resin. Oxidation chemistry and coupling to hydrazide can be used, however, it is understood that any suitable chemical modifications and/or binding interactions that allows specific binding of the carbohydrate moieties of a glycopolypeptide can be used in methods of the invention. The binding interactions of the glycopolypeptides with the solid support are generally covalent, although non-covalent interactions can also be used so long as the glycopolypeptides or glycopeptide fragments remain bound during the digestion, washing and other steps of the methods.

The methods of the invention can also be used to select and characterize subgroups of carbohydrates. Chemical modifications or enzymatic modifications using, for example, glycosidases can be used to isolate subgroups of carbohydrates. For example, the concentration of sodium periodate can be modulated so that oxidation occurs on sialic acid groups of glycoproteins. In particular, a concentration of about 1 mM of sodium periodate at 0.degree. C. can be used to essentially exclusively modify sialic acid groups.

Glycopolypeptides containing specific monosaccharides can be targeted using a selective sugar oxidase to generate aldehyde functions, such as the galactose oxidase described above or other sugar oxidases. Furthermore, glycopolypeptides containing a subgroup of carbohydrates can be selected after the glycopolypeptides are bound to a solid support. For example, glycopeptides bound to a solid support can be selectively released using different glycosidases having specificity for particular monosaccharide structures.

The glycopolypeptides are isolated by binding to a solid support. The solid support can be, for example, a bead, resin, membrane or disk, or any solid support material suitable for methods of the invention. An advantage of using a solid support to bind the glycopolypeptides is that it allows extensive washing to remove non-glycosylated polypeptides. Thus, in the case of complex samples containing a multitude of polypeptides, the analysis can be simplified by isolating glycopolypeptides and removing the non-glycosylated polypeptides, thus reducing the number of polypeptides to be analyzed.

The glycopolypeptides can also be conjugated to an affinity tag through an amine group, such as biotin hydrazide. The affinity tagged glycopeptides can then be immobilized to the solid support, for example, an avidin or streptavidin solid support, and the non-glycosylated peptides are removed. The glycopeptides immobilized on the solid support can be cleaved by a protease, and the non-glycosylated peptide fragments can be removed by washing. The tagged glycopeptides can be released from the solid support by enzymatic or chemical cleavage. Alternatively, the tagged glycopeptides can be released from the solid support with the oligosaccharide and affinity tag attached.

Another advantage of binding the glycopolypeptides to the solid support is that it allows further manipulation of the sample molecules without the need for additional purification steps that can result in loss of sample molecules. For example, the methods of the invention can involve the steps of cleaving the bound glycopolypeptides as well as adding an isotope tag, or other desired modifications of the bound glycopolypeptides. Because the glycopolypeptides are bound, these steps can be carried out on solid phase while allowing excess reagents to be removed as well as extensive washing prior to subsequent manipulations.

The bound glycopolypeptides can be cleaved into peptide fragments to facilitate MS analysis. Thus, a polypeptide molecule can be enzymatically cleaved with one or more proteases into peptide fragments. Exemplary proteases useful for cleaving polypeptides include trypsin, chymotrypsin, pepsin, papain, Staphylococcus aureus (V8) protease, Submaxillaris protease, bromelain, thermolysin, and the like. In certain applications, proteases having cleavage specificities that cleave at fewer sites, such as sequence-specific proteases having specificity for a sequence rather than a single amino acid, can also be used, if desired. Polypeptides can also be cleaved chemically, for example, using CNBr, acid or other chemical reagents. A particularly useful cleavage reagent is the protease trypsin. One skilled in the art can readily determine appropriate conditions for cleavage to achieve a desired efficiency of peptide cleavage.

Cleavage of the bound glycopolypeptides is particularly useful for MS analysis in that one or a few peptides are generally sufficient to identify a parent polypeptide. However, it is understood that cleavage of the bound glycopolypeptides is not required, in particular where the bound glycopolypeptide is relatively small and contains a single glycosylation site. Furthermore, the cleavage reaction can be carried out after binding of glycopolypeptides to the solid support, allowing characterization of non-glycosylated peptide fragments derived from the bound glycopolypeptide. Alternatively, the cleavage reaction can be carried out prior to addition of the glycopeptides to the solid support. One skilled in the art can readily determine the desirability of cleaving the sample polypeptides and an appropriate point to perform the cleavage reaction, as needed for a particular application of the methods of the invention.

Thus, in certain embodiments, glycopeptides are identified as described in Example 14. In this regard, solid phase capture of glycosylated peptides can be achieved either from intact glycoproteins or glycopeptides. In certain embodiments, glycopeptide capture may be preferred since there is no steric hinderance preventing binding of multiple glycosylation sites as can be observed with intact glycoproteins. Another advantage to glycopeptide capture is that hydrophobic membrane proteins generally are not very soluble during glycoprotein capture. However, glycopeptides derived from the same membrane proteins will more likely exhibit favorable solubility thereby enabling enhanced capture.

If desired, the bound glycopolypeptides can be denatured and optionally reduced. Denaturing and/or reducing the bound glycopolypeptides can be useful prior to cleavage of the glycopolypeptides, in particular protease cleavage, because this allows access to protease cleavage sites that can be masked in the native form of the glycopolypeptides. The bound glycopeptides can be denatured with detergents and/or chaotropic agents. Reducing agents such as .beta.-mercaptoethanol, dithiothreitol, tris-carboxyethylphosphine (TCEP), and the like, can also be used, if desired. As discussed above, the binding of the glycopolypeptides to a solid support allows the denaturation step to be carried out followed by extensive washing to remove denaturants that could inhibit the enzymatic or chemical cleavage reactions. The use of denaturants and/or reducing agents can also be used to dissociate protein complexes in which non-glycosylated proteins form complexes with bound glycopolypeptides. Thus, the use of these agents can be used to increase the specificity for glycopolypeptides by washing away non-glycosylated polypeptides from the solid support.

Treatment of the bound glycopolypeptides with a cleavage reagent results in the generation of peptide fragments. Because the carbohydrate moiety is bound to the solid support, those peptide fragments that contain the glycosylated residue remain bound to the solid support. Following cleavage of the bound glycopolypeptides, glycopeptide fragments remain bound to the solid support via binding of the carbohydrate moiety. Peptide fragments that are not glycosylated are released from the solid support. If desired, the released non-glycosylated peptides can be analyzed, as described in more detail below.

The methods of the invention can be used to identify and/or quantify the amount of a glycopolypeptide present in a sample. A particularly useful method for identifying and quantifying a glycopolypeptide is mass spectrometry (MS). The methods of the invention can be used to identify a glycopolypeptide qualitatively, for example, using MS analysis. If desired, an isotope tag can be added to the bound glycopeptide fragments, in particular to facilitate quantitative analysis by MS.

As used herein an “isotope tag” refers to a chemical moiety having suitable chemical properties for incorporation of an isotope, allowing the generation of chemically identical reagents of different mass which can be used to differentially tag a polypeptide in two samples. The isotope tag also has an appropriate composition to allow incorporation of a stable isotope at one or more atoms. A particularly useful stable isotope pair is hydrogen and deuterium, which can be readily distinguished using mass spectrometry as light and heavy forms, respectively. Any of a number of isotopic atoms can be incorporated into the isotope tag so long as the heavy and light forms can be distinguished using mass spectrometry, for example, .sup.13C, .sup.15N, .sup.17O, .sup.18O or .sup.34S. Exemplary isotope tags include the 4,7,10-trioxa-1,13-tridecanediamine based linker and its related deuterated form, 2,2′,3,3′,11,11′,12,12′-octadeutero-4,7,10-trioxa-1,13-t-ridecanediamine, described by Gygi et al. (Nature Biotechnol. 17:994-999 (1999). Other exemplary isotope tags have also been described previously (see WO 00/11208).

In contrast to these previously described isotope tags related to an ICAT-type reagent, it is not required that an affinity tag be included in the reagent since the glycopolypeptides are already isolated. One skilled in the art can readily determine any of a number of appropriate isotope tags useful in methods of the invention. An isotope tag can be an alkyl, akenyl, alkynyl, alkoxy, aryl, and the like, and can be optionally substituted, for example, with O, S, N, and the like, and can contain an amine, carboxyl, sulfhydryl, and the like (see WO 00/11208). Exemplary isotope tags include succinic anhydride, isatoic-anhydride, N-methyl-isatoic-anhydride, glyceraldehyde, Boc-Phe-OH, benzaldehyde, salicylaldehyde, and the like. In addition to Phe and other amino acids similarly can be used as isotope tags. Furthermore, small organic aldehydes can be used as isotope tags. These and other derivatives can be made in the same manner as that disclosed herein using methods well known to those skilled in the art. One skilled in the art will readily recognize that a number of suitable chemical groups can be used as an isotope tag so long as the isotope tag can be differentially isotopically labeled.

The bound glycopeptide fragments are tagged with an isotope tag to facilitate MS analysis. In order to tag the glycopeptide fragments, the isotope tag contains a reactive group that can react with a chemical group on the peptide portion of the glycopeptide fragments. A reactive group is reactive with and therefore can be covalently coupled to a molecule in a sample such as a polypeptide. Reactive groups are well known to those skilled in the art (see, for example, Hermanson, Bioconjugate Techniques, pp. 3-166, Academic Press, San Diego (1996); Glazer et al., Laboratory Techniques in Biochemistry and Molecular Biology: Chemical Modification of Proteins, Chapter 3, pp. 68-120, Elsevier Biomedical Press, New York (1975); Pierce Catalog (1994), Pierce, Rockford Ill.). Any of a variety of reactive groups can be incorporated into an isotope tag for use in methods of the invention so long as the reactive group can be covalently coupled to the immobilized polypeptide.

To analyze a large number or essentially all of the bound glycopolypeptides, it is desirable to use an isotope tag having a reactive group that will react with the majority of the glycopeptide fragments. For example, a reactive group that reacts with an amino group can react with the free amino group at the N-terminus of the bound glycopeptide fragments. If a cleavage reagent is chosen that leaves a free amino group of the cleaved peptides, such an amino group reactive agent can label a large fraction of the peptide fragments. Only those with a blocked N-terminus would not be labeled. Similarly, a cleavage reagent that leaves a free carboxyl group on the cleaved peptides can be modified with a carboxyl reactive group, resulting in the labeling of many if not all of the peptides. Thus, the inclusion of amino or carboxyl reactive groups in an isotope tag is particularly useful for methods of the invention in which most if not all of the bound glycopeptide fragments are desired to be analyzed.

In addition, a polypeptide can be tagged with an isotope tag via a sulfhydryl reactive group, which can react with free sulfhydryls of cysteine or reduced cystines in a polypeptide. An exemplary sulfhydryl reactive group includes an iodoacetamido group (see Gygi et al., supra, 1999). Other examplary sulfhydryl reactive groups include maleimides, alkyl and aryl halides, haloacetyls, .alpha.-haloacyls, pyridyl disulfides, aziridines, acrylolyls, arylating agents and thiomethylsulfones.

A reactive group can also react with amines such as the .alpha.-amino group of a peptide or the .epsilon.-amino group of the side chain of Lys, for example, imidoesters, N-hydroxysuccinimidyl esters (NHS), isothiocyanates, isocyanates, acyl azides, sulfonyl chlorides, aldehydes, ketones, glyoxals, epoxides (oxiranes), carbonates, arylating agents, carbodiimides, anhydrides, and the like. A reactive group can also react with carboxyl groups found in Asp or Glu or the C-terminus of a peptide, for example, diazoalkanes, diazoacetyls, carbonyldiimidazole, carbodiimides, and the like. A reactive group that reacts with a hydroxyl group includes, for example, epoxides, oxiranes, carbonyldiimidazoles, N,N′-disuccinimidyl carbonates, N-hydroxycuccinimidyl chloroformates, and the like. A reactive group can also react with amino acids such as histidine, for example, .alpha.-haloacids and amides; tyrosine, for example, nitration and iodination; arginine, for example, butanedione, phenylglyoxal, and nitromalondialdehyde; methionine, for example, iodoacetic acid and iodoacetamide; and tryptophan, for example, 2-(2-nitrophenylsulfenyl)-3-methyl-3-bromoindolenine (BNPS-skatole), N-bromosuccinimide, formylation, and sulfenylation (Glazer et al., supra, 1975). In addition, a reactive group can also react with a phosphate group for selective labeling of phosphopeptides (Zhou et al., Nat. Biotechnol., 19:375-378 (2001)) or with other covalently modified peptides, including lipopeptides, or any of the known covalent polypeptide modifications. One skilled in the art can readily determine conditions for modifying sample molecules by using various reagents, incubation conditions and time of incubation to obtain conditions suitable for modification of a molecule with an isotope tag. The use of covalent-chemistry based isolation methods is particularly useful due to the highly specific nature of the binding of the glycopolypeptides.

The reactive groups described above can form a covalent bond with the target sample molecule. However, it is understood that an isotope tag can contain a reactive group that can non-covalently interact with a sample molecule so long as the interaction has high specificity and affinity.

Prior to further analysis, it is generally desirable to release the bound glycopeptide fragments. The glycopeptide fragments can be released by cleaving the fragments from the solid support, either enzymatically or chemically. For example, glycosidases such as N-glycosidases can be used to cleave an N-linked carbohydrate moiety and a variety of chemical or other enzymatic reactions can be used to cleave O-linked carbohydrate moieties, and release the corresponding de-glycosylated peptide(s). If desired, N-glycosidases and enzymes or chemicals appropriate for cleavage of O-linked carbohydrate moieties can be added together or sequentially, in either order. The sequential addition of an N-glycosidase and other enzymes for O-linked carbohydrate cleavage allows differential characterization of those released peptides that were N-linked versus those that were O-linked, providing additional information on the nature of the carbohydrate moiety and the modified amino acid residue. Thus, N-linked and O-linked glycosylation sites can be analyzed sequentially and separately on the same sample, increasing the information content of the experiment and simplifying the complexity of the samples being analyzed.

In addition to N-glycosidases, other glycosidases can be used to release a bound glycopolypeptide. For example, exoglycosidases can be used. Exoglycosidases are anomeric, residue and linkage specific for terminal monnosaccharides and can be used to release peptides having the corresponding carbohydrate.

In addition to enzymatic cleavage, chemical cleavage can also be used to cleave a carbohydrate moiety to release a bound peptide. For example, O-linked oligosaccharides can be released specifically from a polypeptide via a .beta.-elimination reaction catalyzed by alkali. The reaction can be carried out in about 50 mM NaOH containing about 1 M NaBH.sub.4 at about 55.degree. C. for about 12 hours. The time, temperature and concentration of the reagents can be varied so long as a sufficient .beta.-elimination reaction is carried out for the needs of the experiment.

In one embodiment, N-linked oligosaccharides can be released from glycopolypeptides, for example, by hydrazinolysis. Glycopolypeptides can be dried in a desiccator over P.sub.2O.sub.5 and NaOH. Anhydrous hydrazine is added and heated at about 100.degree. C. for 10 hours, for example, using a dry heat block.

In addition to using enzymatic or chemical cleavage to release a bound glycopeptide, the solid support can be designed so that bound molecules can be released, regardless of the nature of the bound carbohydrate. The reactive group on the solid support, to which the glycopolypeptide binds, can be linked to the solid support with a cleavable linker. For example, the solid support reactive group can be covalently bound to the solid support via a cleavable linker such as a photocleavable linker. Exemplary photocleavable linkers include, for example, linkers containing o-nitrobenzyl, desyl, trans-o-cinnamoyl, m-nitrophenyl, benzylsulfonyl groups (see, for example, Dorman and Prestwich, Trends Biotech. 18:64-77 (2000); Greene and Wuts, Protective Groups in Organic Synthesis, 2nd ed., John Wiley & Sons, New York (1991); U.S. Pat. Nos. 5,143,854; 5,986,076; 5,917,016; 5,489,678; 5,405,783). Similarly, the reactive group can be linked to the solid support via a chemically cleavable linker. Release of glycopeptide fragments with the intact carbohydrate is particularly useful if the carbohydrate moiety is to be characterized using well known methods, including mass spectrometry. The use of glycosidases to release de-glycosylated peptide fragments also provides information on the nature of the carbohydrate moiety.

Thus, the invention provides methods for identifying a glycopolypeptide and, furthermore, identifying its glycosylation site (“glycosite”). The methods of the invention are applied, as disclosed herein, and the parent glycopolypeptide is identified. The glycosylation site itself can also be identified and consensus motifs determined, as well as the carbohydrate moiety, as disclosed herein. The invention further provides glycopolypeptides, glycopeptides and glycosylation sites identified by the methods of the invention.

Glycopolypeptides from a sample are bound to a solid support via the carbohydrate moiety. The bound glycopolypeptides are generally cleaved, for example, using a,protease, to generate glycopeptide fragments. As discussed above, a variety of methods can be used to release the bound glycopeptide fragments, thereby generating released glycopeptide fragments. As used herein, a “released glycopeptide fragment” refers to a peptide which was bound to a solid support via a covalently bound carbohydrate moiety and subsequently released from the solid support, regardless of whether the released peptide retains the carbohydrate. In some cases, the method by which the bound glycopeptide fragments are released results in cleavage and removal of the carbohydrate moiety, for example, using glycosidases or chemical cleavage of the carbohydrate moiety. If the solid support is designed so that the reactive group, for example, hydrazide, is attached to the solid support via a cleavable linker, the released glycopeptide fragment retains the carbohydrate moiety. It is understood that, regardless whether a carbohydrate moiety is retained or removed from the released peptide, such peptides are referred to as released glycopeptide fragments.

After isolating glycopolypeptides from a sample and cleaving the glycopolypeptide into fragments, the glycopeptide fragments released from the solid support and the released glycopeptide fragments are identified and/or quantified. A particularly useful method for analysis of the released glycopeptide fragments is mass spectrometry. A variety of mass spectrometry systems can be employed in the methods of the invention for identifying and/or quantifying a sample molecule such as a released glycopolypeptide fragment. Mass analyzers with high mass accuracy, high sensitivity and high resolution include, but are not limited to, ion trap, triple quadrupole, and time-of-flight, quadrupole time-of-flight mass spectrometers and Fourier transform ion cyclotron mass analyzers (FT-ICR-MS). Mass spectrometers are typically equipped with matrix-assisted laser desorption (MALDI) and electrospray ionization (ESI) ion sources, although other methods of peptide ionization can also be used. In ion trap MS, analytes are ionized by ESI or MALDI and then put into an ion trap. Trapped ions can then be separately analyzed by MS upon selective release from the ion trap. Fragments can also be generated in the ion trap and analyzed. Sample molecules such as released glycopeptide fragments can be analyzed, for example, by single stage mass spectrometry with a MALDI-TOF or ESI-TOF system. Methods of mass spectrometry analysis are well known to those skilled in the art (see, for example, Yates, J. Mass Spect. 33:1-19 (1998); Kinter and Sherman, Protein Sequencing and Identification Using Tandem Mass Spectrometry, John Wiley & Sons, New York (2000); Aebersold and Goodlett, Chem. Rev. 101:269-295 (2001)).

For high resolution polypeptide fragment separation, liquid chromatography ESI-MS/MS or automated LC-MS/MS, which utilizes capillary reverse phase chromatography as the separation method, can be used (Yates et al., Methods Mol. Biol. 112:553-569 (1999)). Data dependent collision-induced dissociation (CID) with dynamic exclusion can also be used as the mass spectrometric method (Goodlett, et al., Anal. Chem. 72:1112-1118 (2000)).

Once a peptide is analyzed by MS/MS, the resulting CID spectrum can be compared to databases for the determination of the identity of the isolated glycopeptide. Methods for protein identification using single peptides have been described previously (Aebersold and Goodlett, Chem. Rev. 101:269-295 (2001); Yates, J. Mass Spec. 33:1-19 (1998)). In particular, it is possible that one or a few peptide fragments can be used to identify a parent polypeptide from which the fragments were derived if the peptides provide a unique signature for the parent polypeptide. Thus, identification of a single glycopeptide, alone or in combination with knowledge of the site of glycosylation, can be used to identify a parent glycopolypeptide from which the glycopeptide fragments were derived. Further information can be obtained by analyzing the nature of the attached tag and the presence of the consensus sequence motif for carbohydrate attachment. For example, if peptides are modified with an N-terminal tag, each released glycopeptide has the specific N-terminal tag, which can be recognized in the fragment ion series of the CID spectra. Furthermore, the presence of a known sequence motif that is found, for example, in N-linked carbohydrate-containing peptides, that is, the consensus sequence NXS/T, can be used as a constraint in database searching of N-glycosylated peptides.

In addition, the identity of the parent glycopolypeptide can be determined by analysis of various characteristics associated with the peptide, for example, its resolution on various chromatographic media or using various fractionation methods. These empirically determined characteristics can be compared to a database of characteristics that uniquely identify a parent polypeptide, which defines a peptide tag.

The use of a peptide tag and related database is used for identifying a polypeptide from a population of polypeptides by determining characteristics associated with a polypeptide, or a peptide fragment thereof, comparing the determined characteristics to a polypeptide identification index, and identifying one or more polypeptides in the polypeptide identification index having the same characteristics (see WO 02/052259). The methods are based on generating a polypeptide identification index, which is a database of characteristics associated with a polypeptide. The polypeptide identification index can be used for comparison of characteristics determined to be associated with a polypeptide from a sample for identification of the polypeptide. Furthermore, the methods can be applied not only to identify a polypeptide but also to quantify the amount of specific proteins in the sample.

The methods for identifying a polypeptide are applicable to performing quantitative proteome analysis, or comparisons between polypeptide populations that involve both the identification and quantification of sample polypeptides. Such a quantitative analysis can be conveniently performed in two separate stages, if desired. As a first step, a reference polypeptide index is generated representative of the samples to be tested, for example, from a species, cell type or tissue type under investigation, such as a glycopolypeptide sample, as disclosed herein. The second step is the comparison of characteristics associated with an unknown polypeptide with the reference polypeptide index or indices previously generated.

A reference polypeptide index is a database of polypeptide identification codes representing the polypeptides of a particular sample, such as a cell, subcellular fraction, tissue, organ or organism. A polypeptide identification index can be generated that is representative of any number of polypeptides in a sample, including essentially all of the polypeptides potentially expressed in a sample. In methods of the invention directed to identifying glycopolypeptides, the polypeptide identification index is determined for a desired sample such as a serum sample. Once a polypeptide identification index has been generated, the index can be used repeatedly to identify one or more polypeptides in a sample, for example, a sample from an individual potentially having a disease. Thus, a set of characteristics can be determined for glycopeptides that can be correlated with a parent glycopolypeptide, including the amino acid sequence of the glycopeptide, and stored as an index, which can be referenced in a subsequent experiment on a sample treated in substantially the same manner as when the index was generated.

The incorporation of an isotope tag can be used to facilitate quantification of the sample glycopolypeptides. As disclosed previously, the incorporation of an isotope tag provides a method for quantifying the amount of a particular molecule in a sample (Gygi et al., supra, 1999; WO 00/11208). In using an isotope tag, differential isotopes can be incorporated, which can be used to compare a known amount of a standard labeled molecule having a differentially labeled isotope tag from that of a sample molecule. Thus, a standard peptide having a differential isotope can be added at a known concentration and analyzed in the same MS analysis or similar conditions in a parallel MS analysis. A specific, calibrated standard can be added with known absolute amounts to determine an absolute quantity of the glycopolypeptide in the sample. In addition, the standards can be added so that relative quantitation is performed.

Alternatively, parallel glycosylated sample molecules can be labeled with a different isotopic label and compared side-by-side (see Gygi et al., supra, 1999). This is particularly useful for qualitative analysis or quantitative analysis relative to a control sample. For example, a glycosylated sample derived from a disease state can be compared to a glycosylated sample from a non-disease state by differentially labeling the two samples, as described previously (Gygi et al., supra, 1999). Such an approach allows detection of differential states of glycosylation, which is facilitated by the use of differential isotope tags for the two samples, and can thus be used to correlate differences in glycosylation as a diagnostic marker for a disease

As described above, non-glycosylated peptide fragments are released from the solid support after proteolytic or chemical cleavage. The released peptide fragments are then characterized to provide further information on the nature of the glycopolypeptides isolated from the sample. An illustrative method is the use of the isotope-coded affinity tag (ICAT.™.) method (Gygi et al., Nature Biotechnol. 17:994-999 (1999). The ICAT.™. type reagent method uses an affinity tag that can be differentially labeled with an isotope that is readily distinguished using mass spectrometry. The ICAT.™. type affinity reagent consists of three elements, an affinity tag, a linker and a reactive group.

As would be recognized by the skilled artisan, the ICAT.™ reagent is specific for cystine residues. Accordingly, amino-specific reagents are also contemplated for use in the present invention where appropriate. A wide range of reaction principles is available for the derivatization of amino groups. An illustrative method used in proteomics is the acetylation by d0- or d3-acetic acid, thus leading to a light (hydrogenated) or a heavy (deuterated) derivative. The activation of the acetyl group can be achieved, for example, by standard N-hydroxysuccinimide (NHS) chemistry, which leads to high yields of derivatization under smooth conditions. In dependence of the number n of amino groups present in the peptides, mass differences of Δm=3n are introduced by this method. A special case of quantification is realized in the so called iTRAQ- (isobaric tag for relative and absolute quantification) method (Ross, P. L., et al. Mol Cell Proteomics 3 (2004) 1154-69).

In another embodiment, isolated peptides are analyzed to generate three-dimensional (retention time, m/z, and intensity) patterns from LC-MS analysis or an identified peptide patterns from LC-MS/MS analysis and SEQUEST search (11).

The ICAT.™. method or other similar methods can be applied to the analysis of the non-glycosylated peptide fragments released from the solid support. Alternatively, the ICAT.™. method or other similar methods can be applied prior to cleavage of the bound glycopolypeptides, that is, while the intact glycopolypeptide is still bound to the solid support.

In certain embodiments, the method involves the steps of automated tandem mass spectrometry and sequence database searching for peptide/protein identification; stable isotope tagging for quantification by mass spectrometry based on stable isotope dilution theory; and the use of specific chemical reactions for the selective isolation of specific peptides. For example, the previously described ICAT.™. reagent contained a sulfhydryl reactive group, and therefore an ICAT.™.-type reagent can be used to label cysteine-containing peptide fragments released from the solid support. Other reactive groups, as described above, can also be used.

The analysis of the non-glycosylated peptides, in conjunction with the methods of analyzing glycosylated peptides, provides additional information on the state of polypeptide expression in the sample. By analyzing both the glycopeptide fragments as well as the non-glycosylated peptides, changes in glycoprotein abundance as well as changes in the state of glycosylation at a particular glycosylation site can be readily determined.

If desired, the sample can be fractionated by a number of known fractionation techniques. Fractionation techniques can be applied at any of a number of suitable points in the methods of the invention. For example, a sample can be fractionated prior to oxidation and/or binding of glycopolypeptides to a solid support. Thus, if desired, a substantially purified fraction of glycopolypeptide(s) can be used for immobilization of sample glycopolypeptides. Furthermore, fractionation/purification steps can be applied to non-glycosylated peptides or glycopeptides after release from the solid support. One skilled in the art can readily determine appropriate steps for fractionating sample molecules based on the needs of the particular application of methods of the invention.

Methods for fractionating sample molecules are well known to those skilled in the art. Fractionation methods include but are not limited to subcellular fractionation or chromatographic techniques such as ion exchange, including strong and weak anion and cation exchange resins, hydrophobic and reverse phase, size exclusion, affinity, hydrophobic charge-induction chromatography, dye-binding, and the like (Ausubel et al., Current Protocols in Molecular Biology (Supplement 56), John Wiley & Sons, New York (2001); Scopes, Protein Purification: Principles and Practice, third edition, Springer-Verlag, New York (1993)). Other fractionation methods include, for example, centrifugation, electrophoresis, the use of salts, and the like (see Scopes, supra, 1993). In the case of analyzing membrane glycoproteins, well known solubilization conditions can be applied to extract membrane bound proteins, for example, the use of denaturing and/or non-denaturing detergents (Scopes, supra, 1993).

Affinity chromatography can also be used including, for example, dye-binding resins such as Cibacron blue, substrate analogs, including analogs of cofactors such as ATP, NAD, and the like, ligands, specific antibodies useful for immuno-affinity isolation, either polyclonal or monoclonal, and the like. A subset of glycopolypeptides can be isolated using lectin-affinity chromatography, if desired. An exemplary affinity resin includes affinity resins that bind to specific moieties that can be incorporated into a polypeptide such as an avidin resin that binds to a biotin tag on a sample molecule labeled with an ICAT.™.-type reagent. The resolution and capacity of particular chromatographic media are known in the art and can be determined by those skilled in the art. The usefulness of a particular chromatographic separation for a particular application can similarly be assessed by those skilled in the art.

Those of skill in the art will be able to determine the appropriate chromatography conditions for a particular sample size or composition and will know how to obtain reproducible results for chromatographic separations under defined buffer, column dimension, and flow rate conditions. The fractionation methods can optionally include the use of an internal standard for assessing the reproducibility of a particular chromatographic application or other fractionation method. Appropriate internal standards will vary depending on the chromatographic medium or the fractionation method used. Those skilled in the art will be able to determine an internal standard applicable to a method of fractionation such as chromatography. Furthermore, electrophoresis, including gel electrophoresis or capillary electrophoresis, can also be used to fractionate sample molecules.

Tissue-Derived Serum Glycoprotein/Glycosite Sets and Fingerprints

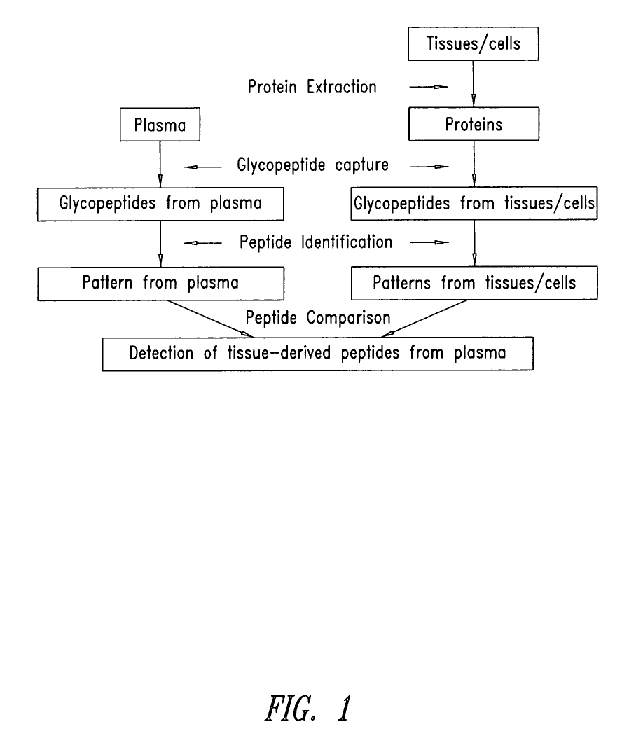

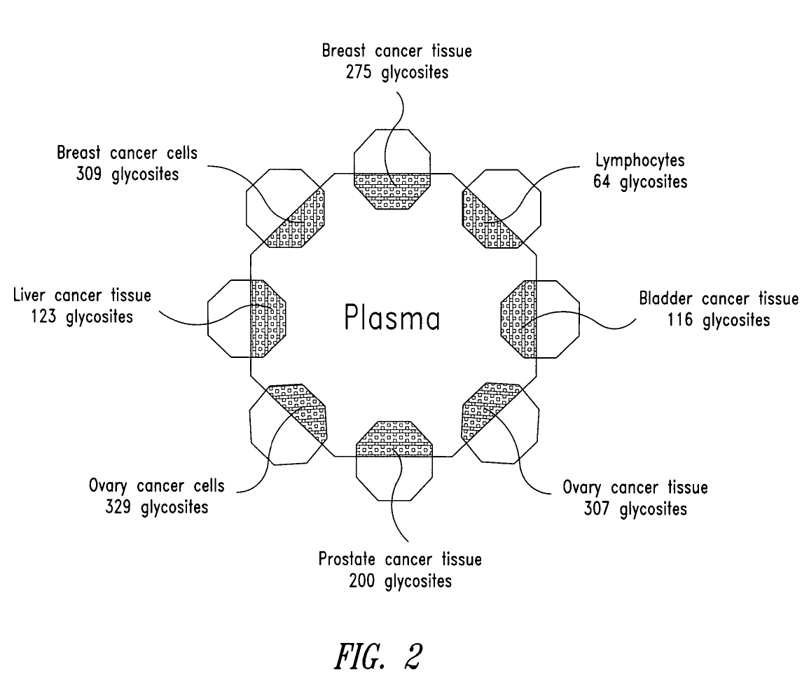

According to the present invention, tissue-derived proteins identified as described herein are compared to plasma-derived proteins identified as described herein to determine overlap between the two (see Example 1). Thus, from the peptides identified from plasma, tissues, or cells, a set of shared peptides and proteins between tissues/cells and plasma are identified (FIG. 2). Illustrative glycoproteins and glycosites of the invention are set forth in Table 1 and SEQ ID NOs:1-11,375; illustrative polynucleotides encoding these glycoproteins are set forth in Table 1 and SEQ ID NOs:11,376-14,917. As outlined in FIG. 1, in one embodiment, the process entails the following: 1) Sample preparation. Cell surface and secreted proteins from tissues/cells and plasma are processed by solid-phase extraction of glylcopeptides (SPEG) as described herein, as well as US Patent Application No 20040023306 and in Zhang, et al., Nature Biotechnology 2003 21:660. Peptides that contain N-linked carbohydrates in the native protein are generally isolated in their de-glycosylated form (8). As would be recognized by the skilled artisan, other similar methods known in the art may be used to isolate glycopeptides from tissue/plasma samples. 2) Pattern generation. Isolated peptides are analyzed to generate three-dimensional (retention time, m/z, and intensity) patterns from LC-MS analysis or an identified peptide patterns from LC-MS/MS analysis and SEQUEST search (11). Other known methods to determine the identity of the isolated peptides may also be used. 3) Pattern analysis. Peptide patterns obtained from different samples are compared and the common peptides from both tissues/cells and plasma are determined (12). 4) Peptide identification. For peptide patterns generated by LC-MS, the common peptides and the proteins from which they originated are identified by tandem mass spectrometry and sequence database searching (FIG. 1).

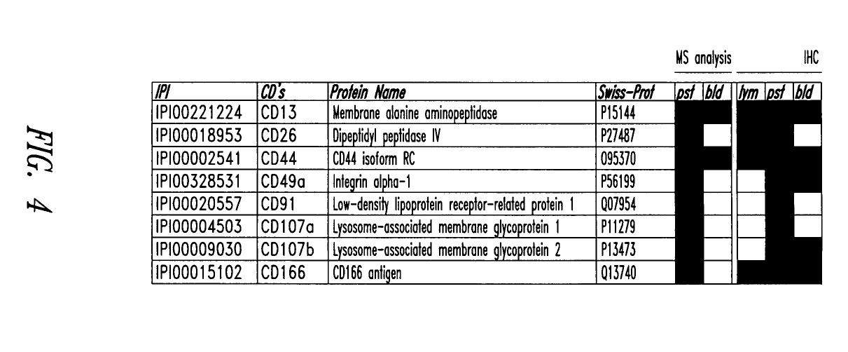

The levels of tissue-derived plasma glycoproteins taken together represent fingerprints in the blood that reflect the operation of normal tissues. While there may be overlap in the tissue expression of certain proteins found in the blood (see e.g., FIG. 4, CD107b, present in the blood and found in prostate and breast), each tissue has a specific normal tissue-derived serum glycoprotein fingerprint (see FIG. 4). When disease attacks a tissue, that blood fingerprint changes, for example, in the levels of these proteins found in the blood and the change in the fingerprint correlates with the specific disease. The changes in the fingerprints occur as a consequence of virtually any disease or tissue perturbation with each disease fingerprint being unique. The changes in the fingerprints are sufficiently informative to carry out disease stratification, follow the progression of the particular disease stratification or type and follow responses to therapy. Measuring the level of glycoproteins that make up a particular tissue-derived serum glycoprotein set in different settings allows one to stratify patients with regard to their ability to respond to particular therapies and even to visualize adverse effects of drugs. The disease-associated fingerprints are determined by comparing the blood from normal individuals against that from patients with specific diseases at known stages. Not only will the absolute levels of the proteins constituting individual fingerprints be determined, but all the protein changes (e.g. N changed proteins) will be compared against one another to generate an N-dimensional shape space that will correlate even more powerfully with the disease stratifications and progression states described above (see e.g., U.S. Patent Application No. 20020095259).

Thus, the present invention is generally directed to methods for identifying tissue-derived glycoproteins present in the blood. The present invention is also directed to methods for defining tissue-derived glycoprotein blood fingerprints and further provides defined examples of tissue-derived glycoprotein blood fingerprints. Additionally, the present invention is directed to panels of reagents or proteomic techniques employing mass spectrometry and other techniques known in the art that detect tissue-derived glycoproteins in the blood for use in diagnostics and other settings.

Thus, the present invention enables the skilled artisan to 1) identify blood glycoproteins which collectively constitute unique molecular blood fingerprints for healthy and diseased individuals; 2) identify unique fingerprints for each different disease; 3) identify fingerprints that can uniquely distinguish the different types of a particular disease (e.g., for prostate cancer, the ability to distinguish between benign disease, slowly growing disease and rapidly metastatic disease); 4 )identify fingerprints that can reveal the stage of progression of each type of disease, and 5) fingerprints that will allow one to assess the response to therapy. The methods for determining the tissue-derived blood fingerprints described herein allow disease detection at very early stages, since even in the earliest disease stages, the cellular networks which control the expression patterns of these blood molecular signatures will be perturbed. Hence the present invention allows detection of virtually any type of disease and detection of each disease at a very early stage.

Normal serum glycoproteins including normal tissue-derived serum glycoproteins are generally identified from a sample of blood collected from a subject using accepted techniques. In one embodiment, blood samples are collected in evacuated serum separator tubes. In another embodiment, blood may be collected in blood collection tubes that contain any anti-coagulant. Illustrative anticoagulants include ethylenediaminetetraacetic acid (EDTA) and lithium heparin. However, any method of blood sample or other bodily fluid or biological/tissue sample collection and storage is contemplated herein. In particular blood may be collected by any portal including the finger, foot, intravenous lines, and portable catheter lines. In one embodiment, blood is centrifuged and the serum layer that separates from the red cells is collected for analysis. In another embodiment, whole blood or plasma is used for analysis.

In certain embodiments a normal blood sample is obtained from human serum recovered from whole blood donations from an FDA-approved clinical source. In this embodiment, the normal, healthy donor hematocrit is between the range of 38% and 55%, the donor weight is over 110 pounds, the donor age is between 18 and 65 years old, the donor blood pressure is in the range of 90-180 mmHg (systolic) and 50-100 mmHg (diastolic), the arms and general appearance of the donor are free of needle marks and any mark signifying risky behavior. The donor pulse should be between 50 bpm-100 bpm, the temperature of the donor should be between 97 and 99.5 degrees. The donor does not have diseases including, but not limited to chest pain, heart disease or lung disease including tuberculosis, cancer, skin disease, any blood disease, or bleeding problems, yellow jaundice, liver disease, hepatitis or a positive test for hepatitis. The donor has not had close contact with hepatitis in the past 12 months nor has the donor ever received pituitary growth hormones.

In certain embodiments, disease free blood is as follows: the donor has not made a donation of blood within the previous 8 weeks, the donor has not had a fever with headache within one week from the date of donation, the donor has not donated a double unit of red cells using an aphaeresis machine within the previous 16 weeks, the donor is not ill with Severe Acute Respiratory Syndrome (SARS), nor has the donor had close contact with someone with SARS, nor has the donor visited (SARS) affected areas. The donor has had no sexual contact with anyone who has HIV/AIDS or has had a positive test for the HIV/AIDS virus, and does not have syphilis or gonorrhea. From 1977 to present, the donor never received money, drugs, or other payment for sex, male donors have never had sexual contact with another male, donors have not had a positive test for the HIV/AIDS virus, donors have not used needles to take drugs, steroids, or anything not prescribed by a physician, donors have not used clotting factor concentrates, donors have not had sexual contact with anyone who was born in or lived in Africa, or traveled to Africa.

Thus, in further embodiments, the present invention provides the normal serum level of components that make up a normal tissue-derived serum glycoprotein set. This level is an average of the levels of a given component measured in a statistically large number of blood samples from normal, healthy individuals. Thus, a “predetermined normal level” is a statistical range of normal and is also referred to herein as “predetermined normal range”. The normal levels or range of levels in the blood for each component are determined by measuring the level of protein in the blood using any of a variety of techiques known in the art and described herein in a sufficient number of blood samples from normal, healthy individuals to determine the standard deviation (SD) with statistically meaningful accuracy.

As would be recognized by the skilled artisan upon reading the present disclosure, in determining the normal serum level of a particular component of a tissue-derived serum glycoprotein set, general biological data is considered and compared, including, for example, gender, time of day of blood sampling, fasting or after food intake, age, race, environment and/or polymorphisms. Biological data may also include data concerning the height, growth rate, cardiovascular status, reproductive status (pre-pubertal, pubertal, post-pubertal, pre-menopausal, menopausal, post-menopausal, fertile, infertile), body fat percentage, and body fat distribution. This list of individual differences that can be measured is exemplary and additional biological data is contemplated.

Thus, the levels of the components that make up a normal tissue-derived serum glycoprotein set are determined. Normal tissue-derived serum glycoprotein fingerprints comprise a data set comprising determined levels in blood from normal, healthy individuals of one, two, three, four, five, six, seven, eight, nine, ten, or more components of a normal tissue-derived serum glycoprotein set. The normal levels in the blood for each component included in a fingerprint are determined by measuring the level of protein in the blood using any of a variety of techniques known in the art and described herein, in a sufficient number of blood samples from normal, healthy individuals to determine the standard deviation (SD) with statistically meaningful accuracy. Thus, as would be recognized by one of skill in the art, a determined normal level is defined by averaging the level of protein measured in a statistically large number of blood samples from normal, healthy individuals and thereby defining a statistical range of normal. A normal tissue-derived serum glycoprotein fingerprint comprises the determined levels in normal, healthy blood of N members of a normal tissue-derived serum glycoprotein set wherein N is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more members up to the total number of members in a given normal tissue-derived serum glycoprotein set. In certain embodiments, a normal tissue-derived serum glycoprotein fingerprint comprises the determined levels in normal, healthy blood of at least two components of a normal tissue-derived serum glycoprotein set. In other embodiments, a normal tissue-derived serum glycoprotein fingerprint comprises the determined levels in normal, healthy blood of at least 3, 4, 5, 6, 7, 8, 9, 10, 11, 12, 13, 14, 15, 16, 17, 18, 19, or 20 components of a normal tissue-derived serum glycoprotein set. In yet further embodiments, a normal control would be run at the time of the assay such that only the presence of a normal sample and the test sample would be necessary and the specific differences between the test sample and the normal sample would then be delineated based upon the panels provided herein.

Each normal tissue controls the expression of a variety of glycoproteins, some of which are expressed at major levels at other tissues in the body and some of which are specifically expressed in the tissue of interest (where specific means that the tissue of interest expresses far more of the glycoprotein than other tissues). Some of the tissue-derived glycoproteins are detected in the blood. Hence a tissue-derived blood fingerprint is comprised of the determined level in the blood of one or more of these tissue-derived glycoproteins. Analysis of levels of these proteins in the blood provides tissue-derived glycoprotein blood fingerprints that are indicative of biological states, including a healthy state or disease states. Thus, there are glycoprotein fingerprints in the blood that reflect the operation of normal tissues and each tissue has a specific glycoprotein fingerprint. These tissue-derived glycoprotein blood fingerprints are perturbed when disease, or other agents such as drugs, affects the tissue. Different diseases will alter the tissue-derived glycoprotein blood fingerprints in different ways. Thus, a unique perturbed glycoprotein blood fingerprint is associated with each type of distinct disease (disease-associated tissue-derived blood fingerprint). In effect, each distinct disease, or stage of a disease, creates its own tissue-derived glycoprotein blood fingerprint for each tissue that it affects. As would be readily appreciated by the skilled artisan, each disease or stage of a disease can affect multiple tissues. For example, in kidney cancer, a primary perturbation in the kidney-derived glycoprotein blood fingerprint would occur. However, a secondary or indirect effect may also be observed in the bladder-derived glycoprotein blood fingerprint. As another example, in liver cancer, perturbation of a liver-derived glycoprotein blood fingerprint as a primary indicator of disease would occur. However, secondary or indirect effects at other sites, for example in a lymphocyte-derived glycoprotein blood fingerprint, would also be observed. As described elsewhere herein, each disease type and stage results in a unique, identifiable blood fingerprint for each tissue that it affects, for primary and secondary tissues affected. Thus, multiple tissue-derived serum glycoprotein sets or components thereof can be measured and used in combination to determine a particular biological state and the blood fingerprints may include the measured level of one or more components derived from the primary tissue affected and/or for a secondary or indirect tissue that is affected by a particular disease.

Most common diseases such as prostate cancer actually represent multiple distinct diseases that initially appear similar (e.g., benign and very slowly growing prostate cancer, slowly invasive prostate cancer and rapidly metastatic prostate cancer represent three different types of prostate cancer—the process of dividing individual prostate cancers into one of these three types is called stratification). The glycoprotein blood fingerprints will be distinct for each of these disease types, thus allowing for the stratification of similar diseases and rapid intervention where necessary. The glycoprotein blood fingerprints will also be perturbed in unique ways as each type of disease progresses—hence the glycoprotein blood fingerprints will also permit the progression of disease to be followed. The glycoprotein blood fingerprints also change with therapy, and hence will permit the effectiveness of therapy to be followed, thereby allowing a physician to alter treatment accordingly. Further, the glycoprotein blood fingerprints change with exposure to a variety of environmental factors, such as drugs, and can be used to assess toxic or off target damage by the drug and it will even permit following the subsequent recovery from such adverse drug exposure.

Thus, a tissue-derived glycoprotein blood fingerprint for a given setting (e.g., a healthy state or a particular disease) is defined by the levels in the blood of the glycoprotein components of a tissue-derived glycoprotein set. As such, a tissue-derived glycoprotein blood fingerprint for a given tissue at any given time and in any given disease setting is determined by measuring the levels of each of a plurality of tissue-derived glycoproteins in the blood. It is the combination of the different levels in the blood of the tissue-derived glycoproteins that make up the tissue-derived glycoprotein set that reveals a unique pattern that defines the fingerprint. Equally important, each of the levels of the proteins can be compared against one another to create an N-dimensional measure of the fingerprint space, a very powerful correlate to health and disease (see e.g., U.S. Patent Application No 20020095259).

As such, a tissue-derived glycoprotein blood fingerprint may comprise the determined level in the blood of anywhere from about 2 to more than about 100, 200 or more tissue-derived glycoproteins derived from a particular tissue or tissues of interest. In one embodiment, the tissue-derived glycoprotein blood fingerprint comprises the quantitatively measured level in the blood of at least 3, 4, 5, 6, 7, 8, 9, or 10 tissue-derived glycoproteins derived from a particular tissue of interest. In another embodiment, the tissue-derived glycoprotein blood fingerprint comprises the determined level in the blood of at least 11, 12, 13, 14, 15, 16, 17, 18, 19, 20, 21, 22, 23, 24, 25, 26, 28, 29, or 30 tissue-derived glycoproteins derived from a particular tissue of interest. In a further embodiment, the tissue-derived glycoprotein blood fingerprint comprises the determined level in the blood of at least, 31, 32, 33, 34, 35, 36, 37, 38, 39, or 40 tissue-derived glycoproteins derived from a particular tissue of interest. In yet a further embodiment, the tissue-derived glycoprotein blood fingerprint comprises the determined level in the blood of at least, 41, 42, 43, 44, 45, 46, 47, 48, 49, or 50 tissue-derived glycoproteins derived from a particular tissue of interest. In an additional embodiment, the tissue-derived glycoprotein blood fingerprint comprises the determined level in the blood of 51, 52, 53, 54, 55, 56, 57, 58, 59, or 60 tissue-derived glycoproteins derived from a particular tissue of interest. In another embodiment, the tissue-derived glycoprotein blood fingerprint comprises the determined level in the blood of 61, 62, 63, 64, 65, 66, 67, 68, 69, or 70 tissue-derived glycoproteins derived from a particular tissue of interest. In further embodiments, the tissue-derived glycoprotein blood fingerprint comprises the determined level in the blood of 75, 80, 85, 90, 100, or more tissue-derived glycoproteins derived from a particular tissue of interest.

In one embodiment, a prostate-derived glycoprotein blood fingerprint comprises the determined level in the blood of any one or more of the following glycoproteins: CD91, CD107a, CD143, PSMA-1, and tumor endothelial marker 7-related precursor (see Table 1 and FIG. 4). In a further embodiment, a prostate-derived glycoprotein blood fingerprint comprises the determined level in the blood of any one or more of the following glycoproteins: CD13, CD14, CD26, CD44, CD45, CD56, CD90, CD91, CD107a, CD107b, CD109, CD166, CD143, CD224, PSMA-1, Glutamate carboxypeptidase II, MAC-2 binding protein, metalloproteinase inhibitor 1, and tumor endothelial marker 7-related precursor (see Table 1 and FIG. 4).

In one embodiment, a lymphocyte-derived glycoprotein blood fingerprint comprises the determined level in the blood of any one or more of the following glycoproteins: CD2, CD21, CD49d, CD50, CD62L, CD102, CD124, and interferon-alpha/beta receptor beta chain. In a further embodiment, a lymphocyte-derived glycoprotein blood fingerprint comprises the determined level in the blood of any one or more of the following glycoproteins: CD2, CD13, CD21, CD44, CD45, CD49c, CD49d, CD50, CD54, CD56, CD62L, CD71, CD74, CD90, CD98, CD109, CD166, CD102, CD124, CD224, MAC-2 binding protein, and interferon-alpha/beta receptor beta chain.

In one embodiment, a bladder-derived glycoprotein blood fingerprint comprises the determined level in the blood of any one or more of the following glycoproteins: CD13, CD44, CD56, MAC2-binding protein, and metalloproteinase inhibitor 1.

In another embodiment, a breast-derived glycoprotein blood fingerprint comprises the determined level in the blood of any one or more of the following glycoproteins: CD71, CD98, CD107b, CD155, CD224, MAC-2 binding protein, receptor protein-tyrosine kinase erbB-2, and tumor-associated calcium signal transducer 2. In a further embodiment, a breast-derived glycoprotein blood fingerprint comprises the determined level in the blood of any one or more of the following glycoproteins: CD155, receptor protein-tyrosine kinase erbB-2, and tumor-associated calcium signal transducer 2.

In one embodiment, a liver-derived glycoprotein blood fingerprint comprises the determined level in the blood of any one or more of the following glycoproteins: CD13, CD14, CD44, CD54, CD56, CD90, CD166, MAC-2 binding protein, metalloproteinase inhibitor 1, and receptor protein-tyrosine kinase erbB-4.

It should be noted that in certain circumstances, a tissue-derived glycoprotein blood fingerprint can be defined (in part or entirely) merely by the presence or absence of one or a plurality of tissue-derived glycoproteins, and determining the exact level of each of a plurality of tissue-derived glycoproteins in the blood may not be necessary.

In a further embodiment, the disease-associated (e.g., perturbed) tissue-derived glycoprotein blood fingerprints for a particular tissue are determined by comparing the blood from normal individuals against that from patients with specific diseases at known stages. Thus, the disease-associated fingerprint is a data set comprising the determined level in a blood sample from an individual afflicted with a disease of one or more components of a normal tissue-derived serum glycoprotein set that demonstrates a statistically significant change as compared to the determined normal level (e.g., wherein the level in the disease sample is above or below a predetermined normal range). The data set is compiled from samples from individuals who are determined to have a particular disease using established medical diagnostics for the particular disease. The blood (serum) level of each protein member of a normal tissue-derived serum glycoprotein set as measured in the blood of the diseased sample is compared to the corresponding determined normal level. A statistically significant variation from the determined normal level for one or more members of the normal serum tissue-derived protein set provides diagnostically useful information (disease-associated fingerprint) for that disease. Note that it may be determined for a particular disease or disease state that the level of only a few members of the normal tissue-derived serum protein set change relative to the normal levels. Thus, a disease-associated tissue-derived blood fingerprint may comprise the determined levels in the blood of only a subset of the components of a normal tissue-derived serum glycoprotein set for a given tissue and a particular disease. Thus, a disease-associated tissue-derived blood fingerprint comprises the determined levels in blood (or as noted herein any bodily fluid or tissue sample, however in most embodiments samples from blood are compared with a normal from blood and so on) of N members of a tissue-derived serum glycoprotein set wherein N is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 110 or more or any integer value therebetween., or more members up to the total number of members in a given tissue-derived serum glycoprotein set tissue-derived serum glycoprotein set. In this regard, in certain embodiments, a disease-associated tissue-derived blood fingerprint comprises the determined levels of one or more components of a normal tissue-derived serum glycoprotein set. In one embodiment, a disease-associated tissue-derived blood fingerprint comprises the determined levels of at least two components of a normal tissue-derived serum glycoprotein set. In other embodiments, a disease-associated tissue-derived blood fingerprint comprises the determined levels of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 110 or more or any integer value therebetween components of a normal tissue-derived serum glycoprotein set.

The skilled artisan would readily appreciate that a variety of statistical tests can be used to determine if an altered level of a given protein is significant. The Z-test (Man, M. Z., et al., Bioinformatics, 16: 953-959, 2000) or other appropriate statistical tests can be used to calculate P values for comparison of protein expression levels.

Tissue-derived glycoprotein blood fingerprints can be determined using any of a variety of detection reagents such as described herein and known in the art in the context of a variety of methods for measuring protein levels known in the art and described herein. Any detection reagent that can specifically bind to or otherwise detect tissue-derived glycoproteins as described herein is contemplated as a suitable detection reagent. Illustrative detection reagents are described elsewhere herein and include, but are not limited to antibodies, or antigen-binding fragments thereof, yeast ScFv, DNA or RNA aptamers, isotope labeled peptides, microfluidic/nanotechnology measurement devices and the like.

Methods for measuring tissue-derived glycoprotein levels from blood/serum/plasma include, but are not limited to, immunoaffinity based assays such as ELISAs, Western blots, and radioimmunoassays, and mass spectrometry based methods (matrix-assisted laser desorption ionization (MALDI), MALDI-Time-of-Flight (TOF), Tandem MS (MS/MS), electrospray ionization (ESI), Surface Enhanced Laser Desorption Ionization (SELDI)-TOF MS, liquid chromatography (LC)-MS/MS, etc). Other methods useful in this context include isotope-coded affinity tag (ICAT) followed by multidimensional chromatography and MS/MS. The procedures described herein for analysis of blood tissue-derived glycoprotein fingerprints can be modified and adapted to make use of microfluidics and nanotechnology in order to miniaturize, parallelize, integrate and automate diagnostic procedures (see e.g., U.S. Patent Application Nos. 20040023306, 20050095649, and 20060141528; L. Hood, et al., Science 306:640-643; R. H. Carlson, et al., Phys. Rev. Lett. 79:2149 (1997); A. Y. Fu, et al., Anal. Chem. 74:2451 (2002); J. W. Hong, et al., Nature Biotechnol. 22:435 (2004); A. G. Hadd, et al., Anal. Chem. 69:3407 (1997); I. Karube, et al., Ann. N.Y. Acad. Sci. 750:101 (1995); L. C. Waters et al., Anal. Chem. 70:158 (1998); J. Fritz et al., Science 288, 316 (2000)).

It should be noted that when the term “blood” is used herein, any part of the blood is intended. Accordingly, for determining tissue-derived glycoprotein blood fingerprints, whole blood may be used directly where appropriate, or plasma or serum may be used.

As one of skill in the art could readily appreciate any number of methodologies can be employed to investigate the tissue-derived nucleic acid and polypeptide sequences set forth by the present invention. In addition to protein or nucleic acid array or microarray analysis, other nanoscale analysis may be employed. Such methodologies include, but are not limited to microfluidic platforms, nanowire sensors (Bunimovich et al., Electrocheically Programmed, Spatially Selective Biofunctionalization of Silicon Wires, Langmuir 20, 10630-10638, 2004; Curreli et al., J. Am. Chem. Soc. 127, 6922-6923, 2005). Further, the use of high-affinity protein-capture agents is contemplated. Such capture agents may include DNA aptamers (U.S. Patent Application Pub. No. 20030219801, as well as the use of click chemistry for target-guided synthesis (Lewis et al., Angewandte Chemie-International Edition, 41, 1053-, 2002; Manetsch et al., J. Am. Chem. Soc. 126, 12809-12818, 2004; Ramstrom et al., Nature Rev. Drug Discov. 1, 26-36, 2002).

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, New York, Gait, “Oligonucleotide Synthesis: A Practical Approach” 1984, IRL Press, London, Nelson and Cox (2000), Lehninger, Principles of Biochemistry ³rd Ed., W. H. Freeman Pub., New York, N.Y. and Berg et al. (2002) Biochemistry, 5th Ed., W. H. Freeman Pub., New York, N.Y., all of which are herein incorporated in their entirety by reference for all purposes.

As would be recognized by the skilled artisan, while the tissue- and/or serum-derived glycoproteins, the levels of which make up a given normal or disease-associated fingerprint, need not be isolated, in certain embodiments, it may be desirable to isolate such proteins (e.g., for antibody production or for developing other detection reagents as described herein). As such, the present invention provides for isolated tissue- and/or serum-derived glycoproteins or fragments or portions thereof and polynucleotides that encode such proteins. As used herein, the terms protein and polypeptide are used interchangeably. Also, the isolated glycoproteins may not remain glycoproteins when isolated as isolation may remove glycosylation. Illustrative (glyco)proteins include those provided in the amino acid sequences set forth in in the appended sequence listing. The terms polypeptide and protein encompass amino acid chains of any length, including full-length endogenous (i.e., native) proteins and variants of endogenous polypeptides described herein. Variants are polypeptides that differ in sequence from the polypeptides of the present invention only in substitutions, deletions and/or other modifications, such that either the variants disease-specific expression patterns are not significantly altered or the polypeptides remain useful for diagnostics/detection of glycoproteins and glycosites as described herein. For example, modifications to the polypeptides of the present invention may be made in the laboratory to facilitate expression and/or purification and/or to improve immunogenicity for the generation of appropriate antibodies and other detection agents. Modified variants (e.g., chemically modified) of the (glyco)proteins may be useful herein, (e.g., as standards in mass spectrometry analyses of the corresponding proteins in the blood, and the like). As such, in certain embodiments, the biological function of a variant protein is not relevant for utility in the methods for detection and/or diagnostics described herein. Polypeptide variants generally encompassed by the present invention will typically exhibit at least about 70%, 75%, 80%, 85%, 86%, 87%, 88%, 89%, 90%, 91%, 92%, 93%, 94%, 95%, 96%, 97%, 98%, or 99% or more identity along its length, to a polypeptide sequence set forth herein. Within a polypeptide variant, amino acid substitutions are usually made at no more than 50% of the amino acid residues in the native polypeptide, and in certain embodiments, at no more than 25% of the amino acid residues. In certain embodiments, such substitutions are conservative. A conservative substitution is one in which an amino acid is substituted for another amino acid that has similar properties, such that one skilled in the art of peptide chemistry would expect the secondary structure and hydropathic nature of the polypeptide to be substantially unchanged. In general, the following amino acids represent conservative changes: (1) ala, pro, gly, glu, asp, gin, asn, ser, thr; (2) cys, ser, tyr, thr; (3) val, ile, leu, met, ala, phe; (4) lys, arg, his; and (5) phe, tyr, trp, his. Thus, a variant may comprise only a portion of a native polypeptide sequence as provided herein. In addition, or alternatively, variants may contain additional amino acid sequences (such as, for example, linkers, tags and/or ligands), usually at the amino and/or carboxy termini. Such sequences may be used, for example, to facilitate purification, detection or cellular uptake of the polypeptide.

When comparing polypeptide sequences, two sequences are said to be identical if the sequence of amino acids in the two sequences is the same when aligned for maximum correspondence, as described below. Comparisons between two sequences are typically performed by comparing the sequences over a comparison window to identify and compare local regions of sequence similarity. A comparison window as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, Wis.), using default parameters. This program embodies several alignment schemes described in the following references: Dayhoff, M. O. (1978) A model of evolutionary change in proteins Matrices for detecting distant relationships. In Dayhoff, M. O. (ed.) Atlas of Protein Sequence and Structure, National Biomedical Research Foundation, Washington D.C. Vol. 5, Suppl. 3, pp. 345-358; Hein J. (1990) Unified Approach to Alignment and Phylogenes pp. 626-645 Methods in Enzymology vol. 183, Academic Press, Inc., San Diego, Calif.; Higgins, D. G. and Sharp, P. M. (1989) CABIOS 5:151-153; Myers, E. W. and Muller W. (1988) CABIOS 4:11-17; Robinson, E. D. (1971) Comb. Theor 11:105; Saitou, N. Nei, M. (1987) Mol. Biol. Evol. 4:406-425; Sneath, P. H. A. and Sokal, R. R. (1973) Numerical Taxonomy the Principles and Practice of Numerical Taxonomy, Freeman Press, San Francisco, Calif.; Wilbur, W. J. and Lipman, D. J. (1983) Proc. Natl. Acad., Sci. USA 80:726-730.

Alternatively, optimal alignment of sequences for comparison may be conducted by the local identity algorithm of Smith and Waterman (1981) Add. APL. Math 2:482, by the identity alignment algorithm of Needleman and Wunsch (1970) J. Mol. Biol. 48:443, by the search for similarity methods of Pearson and Lipman (1988) Proc. Natl. Acad. Sci. USA 85: 2444, by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

Illustrative examples of algorithms that are suitable for determining percent sequence identity and sequence similarity include the BLAST and BLAST 2.0 algorithms, which are described in Altschul et al. (1977) Nucl. Acids Res. 25:3389-3402 and Altschul et al. (1990) J. Mol. Biol. 215:403-410, respectively. BLAST and BLAST 2.0 can be used, for example, to determine percent sequence identity for the polynucleotides and polypeptides of the invention. Software for performing BLAST analyses is publicly available through the National Center for Biotechnology Information.

An isolated polypeptide is one that is removed from its original environment. For example, a naturally occurring protein or polypeptide is isolated if it is separated from some or all of the coexisting materials in the natural system. In certain embodiments, such polypeptides are also purified, e.g., are at least about 90% pure by weight of protein in the preparation, in some embodiments, at least about 95% pure by weight of protein in the preparation and in further embodiments, at least about 99% pure by weight of protein in the preparation.

In one embodiment of the present invention, a polypeptide comprises a fusion protein comprising a glycopolypeptide or glycosite as described herein. The present invention further provides fusion proteins that comprise at least one polypeptide as described herein, as well as polynucleotides encoding such fusion proteins. The fusion proteins may comprise multiple polypeptides or portions/variants thereof, as described herein, and may further comprise one or more polypeptide segments for facilitating the expression, purification, detection, and/or activity of the polypeptide(s).

In certain embodiments, the proteins and/or polynucleotides, and/or fusion proteins are provided in the form of compositions, e.g., pharmaceutical compositions, vaccine compositions, compositions comprising a physiologically acceptable carrier or excipient. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

In certain embodiments, wash buffer refers to a solution that may be used to wash and remove unbound material from an adsorbent surface. Wash buffers typically include salts that may or may not buffer pH within a specified range, detergents and optionally may include other ingredients useful in removing adventitiously associated material from a surface or complex.

In certain embodiments, elution buffer refers to a solution capable of dissociating a binding moiety and an associated analyte. In some circumstances, an elution buffer is capable of disrupting the interaction between subunits when the subunits are associated in a complex. As with wash buffers, elution buffers may include detergents, salt, organic solvents and may be used separately or as mixtures. Typically, these latter reagents are present at higher concentrations in an elution buffer than in a wash buffer making the elution buffer more disruptive to molecular interactions. This ability to disrupt molecular interactions is termed “stringency,” with elution buffers having greater stringency that wash buffers.

In general, tissue- and/or serum-derived glycopolypeptides and polynucleotides encoding such polypeptides as described herein, may be prepared using any of a variety of techniques that are well known in the art. For example, a polynucleotide encoding a protein may be prepared by amplification from a suitable cDNA or genomic library using, for example, polymerase chain reaction (PCR) or hybridization techniques. Libraries may generally be prepared and screened using methods well known to those of ordinary skill in the art, such as those described in Sambrook et al., Molecular Cloning: A Laboratory Manual, Cold Spring Harbor Laboratories, Cold Spring Harbor, N.Y., 1989. cDNA libraries may be prepared from any of a variety of organs, tissues, cells, as described herein. Other libraries that may be employed will be apparent to those of ordinary skill in the art upon reading the present disclosure. Primers for use in amplification may be readily designed based on the polynucleotide sequences encoding polypeptides as provided herein, for example, using programs such as the PRIMER3 program (see website: http colon double slash www dash genome dot wi dot mit dot edu slash cgi dash bin slash primer slash primer3 www dot cgi).

Diagnostic/Prognostic Panels

The normal tissue-derived serum glycoprotein and glycosite sets defined herein and the predetermined normal levels of the components that make up the tissue-derived serum glycoprotein or glycosite sets (e.g., the database of predetermined normal serum levels of tissue-derived glycoproteins or glycosites) can be used as a baseline against which one can determine any perturbation of the normal state. Perturbation of the normal biological state is identified by measuring levels of tissue-derived serum glycoproteins or glycosites from a patient and comparing the measured levels against the predetermined normal levels. Any level that is statistically significantly altered from the normal level (i.e., any level from the disease sample that is outside (either above or below) the predetermined normal range) indicates a perturbation of normal and thus, the presence of disease (or effect of a drug or environmental agent, etc.). In this way, the predetermined normal levels of normal tissue-derived serum glycoproteins or glycosites are also used to identify and define disease-associated tissue-derived blood fingerprints. The diagnostic/prognostic panels of the present invention typically comprise detection reagents for detecting proteins, glycosites, or nucleic acid molecules that are tissue-derived glycoproteins, but that may be found in a bodily fluid such as blood, urine, saliva, etc. or a tissue sample.

As used herein, a panel may detect less than the entire set of tissue-derived glycoprotein sequences, or the polynucleotides that encode these proteins, as defined in the tables herein (see e.g., Table 1) for a given tissue. For example, as can be readily appreciated by the skilled artisan, measuring the level of 1 transcript or protein of each tissue may be enough to generally monitor the health of a tissue. However, increasing the number of probes targeting the component (nucleic acid or polypeptide), while not necessary, will add specificity and sensitivity to the assay. Accordingly, in certain aspects at least 5 probes per tissue-derived serum glycoprotein set will be present in the panel, in other aspects at least 10 probes per tissue-derived serum glycoprotein set will be present, yet in others there may be 20, 30, 40, 50 or more probes present per tissue-derived serum glycoprotein set. In certain embodiments, probes per set may include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 110 or any integer value therebetween.

Thus, the present invention provides panels for detecting and measuring the level of tissue-derived glycoproteins and glycosites in serum that can be used in a variety of diagnostic settings. Illustrative glycoproteins and glycosites of the invention are set forth in Table 1 and SEQ ID NOs:1-11,375; illustrative polynucleotides encoding these glycoproteins are set forth in Table 1 and SEQ ID NOs:11,376-14,917. As used herein and discussed further below, “diagnostic panel or prognostic panel” is meant to encompass panels, arrays, mixtures, and kits that may comprise detection reagents or probes specific to a tissue-derived glycoprotein component or a control (control nucleic acid or polypeptide sequences may or may not be a component of a tissue-derived serum glycoprotein set) and any of a variety of associated buffers, solutions, appropriate negative and positive controls, instruction sets, and the like. In certain embodiments, a detection reagent may comprise antibodies (or antigen-binding fragments thereof) either with a secondary detection reagent attached thereto or without, nucleic acid probes, aptamers, click reagents, etc. Further, a “panel” may comprise panels, arrays, mixtures, kits, or other arrangements of proteins, antibodies or antigen-binding fragments thereof to tissue-derived serum glycoproteins, nucleic acid molecules encoding tissue-derived serum glycoproteins, nucleic acid probes that hybridize to nucleic acid sequences encoding tissue-derived serum glycoproteins. Moreover, a panel may be derived from only one tissue or two, three, four, five, six, seven, eight, or more tissues. Certain biological systems such as the cardiovascular system or the central nervous system, comprise numerous tissues. Thus, in certain embodiments, numerous such tissues may be grouped together in a single panel.

The present invention also provides panels for detecting the tissue-derived serum glycoproteins at any given time in a subject. The term “subject” is intended to include any mammal or indeed any vertebrate that may be used as a model system for human disease. Examples of subjects include humans, monkeys, apes, dogs, cats, mice, rats, zebra fish, and transgenic species thereof.

The panels are comprised of a plurality of detection reagents (e.g., at least two) that each specifically detects a tissue-derived serum glycoprotein, or a transcript encoding such a protein), wherein the levels of tissue-derived glycoproteins in blood derived from a particular tissue taken together form a unique pattern that defines the fingerprint. In certain embodiments, detection reagents can be bispecific such that the panel is comprised of a plurality of bispecific detection reagents that may specifically detect more than one tissue-derived blood glycoprotein. The term “specifically” is a term of art that would be readily understood by the skilled artisan to mean, in this context, that the protein or proteins of interest is/are detected by the particular detection reagent but other unrelated proteins are not significantly detected. Specificity can be determined using appropriate positive and negative controls and by routinely optimizing conditions. In certain embodiments, detection reagents specifically detect one or more members of a family of related proteins (or polynucleotides encoding such proteins) but do not significantly detect other unrelated control proteins or transcripts. Thus, as would be understood by the skilled artisan, detection reagents may specifically detect a single variant protein or transcript or may specifically detect a group of related proteins or transcripts encoding such proteins.

The diagnostic panels of the present invention comprise detection reagents wherein each detection reagent binds to one tissue-derived serum glycoprotein. As discussed elsewhere herein, in certain embodiments, the detection reagent may bind to one glycosite present in one or more tissue-derived serum glycoptroteins. As noted above, panels may also comprise controls that are not or may not be specific for a particular tissue-derived protein or transcript. In certain embodiments, the detection reagents of a panel can each bind to tissue-derived proteins from one tissue-derived serum glycoprotein set or from more than one tissue-derived serum glycoprotein set. For example, a particular diagnostic panel may comprise detection reagents that together detect one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, thirty, thirty-one, thirty-two, thirty-three, thirty-four, thirty-five, thirty-six, thirty-seven, thirty-eight, thirty-nine, forty, forty-one, forty-two, forty-three, forty-four, forty-five, forty-six, forty-seven, forty-eight, forty-nine, fifty, sixty, seventy, eighty, ninety, one-hundred or more tissue-derived serum glycoproteins, such as those provided in Table 1. In particular, a diagnostic panel may comprise detection reagents that detect one or more prostate-derived serum glycoproteins or one or more bladder-derived serum glycoproteins as listed in Table 1.

It should be noted that in certain embodiments, the tissue-derived glycoproteins and glycosites as listed in Table 1 that do not overlap with the normal serum glycoprotein or glycosite set are also useful diagnostically. For example, two prostate cancer tissue proteins, prostatic acid phosphatase (PAP) and prostate-specific antigen (PSA) were not found in the plasma dataset. However, the levels of these proteins have been shown to be elevated in the plasma of prostate cancer patients and are unlikely to be detected in plasma of normal donors (Ludwig J A, Weinstein J N. (2005) Biomarkers in cancer staging, prognosis and treatment selection. Nat Rev Cancer 5: 845-856). Accordingly, the present invention also contemplates diagnostic/prognostic panels that detect one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, thirty, thirty-one, thirty-two, thirty-three, thirty-four, thirty-five, thirty-six, thirty-seven, thirty-eight, thirty-nine, forty, forty-one, forty-two, forty-three, forty-four, forty-five, forty-six, forty-seven, forty-eight, forty-nine, fifty, sixty, seventy, eighty, ninety, one-hundred or more tissue-derived glycoproteins, wherein the tissue-derived glycoproteins are derived from the same tissue, such as those listed in Table 1 (e.g., prostate-derived glycoproteins, bladder-derived glycoproteins, ovary-derived glycoproteins, breast-derived glycoproteins, lymphocyte-derived glycoproteins, etc.).

In certain embodiments, the diagnostic/prognostic panels of the present invention comprise detection reagents that specifically bind to the identified glycosites described in Table 1. In this regard, the identified glycosites may map to more than one glycoprotein in the public databases. In other words, multiple glycoproteins contain the same glycosite. Thus, in certain embodiments, it is not necessary to measure the levels of a single glycoprotein that contains the glycosite; it is sufficient to detect and measure the level of all proteins that contain a given glycosite by using detection reagents the specifically bind to the glycosite itself. Differential glycoprotein levels determined in this manner are useful in a variety of diagnostic settings. Thus, the panels of the present invention may comprise detection reagents that bind to one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, thirty, thirty-one, thirty-two, thirty-three, thirty-four, thirty-five, thirty-six, thirty-seven, thirty-eight, thirty-nine, forty, forty-one, forty-two, forty-three, forty-four, forty-five, forty-six, forty-seven, forty-eight, forty-nine, fifty, sixty, seventy, eighty, ninety, one-hundred or more glycosites, wherein the tissue-derived glycosites are derived from the same tissue, such as those listed in Table 1.

Panels of the invention comprise N detection reagents wherein N is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more detection reagents up to the total number of members in a given glycoprotein or glycosite set that are to be detected. As noted above, in certain embodiments, it may be desirable to detect proteins from two or more tissue-derived serum glycoprotein sets. Accordingly, the diagnostic panels of the invention may comprise N detection reagents wherein N is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more detection reagents up to the total number of members in one or more tissue-derived serum glycoprotein sets that are to be detected. Detection reagents of a given diagnostic panel may detect proteins from 1, 2, 3, 4, 5, 6, 7, 8, 9, 10, 11, 12, 13, 14, 15, 16, 17, 18, 19, 20, or more tissue-derived serum glycoprotein sets, such as those provided in Table 1, or normal serum tissue-derived glycoprotein sets thereof.

In certain embodiments, the detection reagents for a diagnostic panel are selected such that the level of at least one of the tissue-derived serum glycoprotein detected by the plurality of detection reagents in a blood sample from a subject afflicted with a disease affecting the tissue or tissues from which the tissue-derived serum glycoprotein are derived is above or below a predetermined normal range. In certain embodiments, the detection reagents for a diagnostic panel are selected such that the level of at least two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, thirty, thirty-one, thirty-two, thirty-three, thirty-four, thirty-five, thirty-six, thirty-seven, thirty-eight, thirty-nine, forty, forty-one, forty-two, forty-three, forty-four, forty-five, forty-six, forty-seven, forty-eight, forty-nine, fifty, sixty, seventy, eighty, ninety, one-hundred or more of the tissue-derived serum glycoprotein detected by the plurality of detection reagents in a biological sample (e.g., blood) from a subject afflicted with a disease affecting the tissue or tissues from which the glycoproteins are derived is above or below a predetermined normal range. Thus, the detection reagents for a diagnostic panel, kit, or array may be selected such that the level of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45,4 6, 47, 48, 49, 50, 60, 70, 80, 90, 100, 110 or any integer value therebetween, or more of the tissue-derived and/or serum glycoproteins or glycosites detected by the plurality of detection reagents in a blood sample from a subject afflicted with a disease affecting the tissue or tissues from which the tissue-derived serum glycoprotein are derived is above or below a predetermined normal range.

Tissue-derived and/or serum glycoproteins or glycosites can be detected and measured using any of a variety of detection reagents in the context of a variety of methods for quantifying protein levels. Any detection reagent that can specifically bind to or otherwise detect a tissue-derived glycoprotein as described herein is contemplated as a suitable detection reagent. Illustrative detection reagents include, but are not limited to antibodies, or antigen-binding fragments thereof, oligopeptides, polynucleotides, oligonucleotide probes/primers, binding organic molecules, yeast ScFv, DNA or RNA aptamers, isotope labeled peptides, receptors, ligands, click reagents, molecular beacons, quantum dots, microfluidic/nanotechnology measurement devices and the like. The “detection reagents” of the present invention may comprise methods for detecting and quantifying proteins, such mass spectrometry based methods (matrix-assisted laser desorption ionization (MALDI), MALDI-Time-of-Flight (TOF), Tandem MS (MS/MS), electrospray ionization (ESI), Surface Enhanced Laser Desorption Ionization (SELDI)-TOF MS, liquid chromatography (LC)-MS/MS, etc). Other methods useful in this context include isotope-coded affinity tag (ICAT) followed by multidimensional chromatography and MS/MS.

The detection reagents of the present invention may comprise any of a variety of detectable labels or reporter groups. The invention contemplates the use of any type of detectable label, including, e.g., visually detectable labels, fluorophores, and radioactive labels. The detectable label may be incorporated within or attached, either covalently or non-covalently, to the detection reagent. Detectable labels or reporter groups may include radioactive groups, dyes, fluorophores, biotin, colorimetric substrates, enzymes, or colloidal compounds. Illustrative detectable labels or reporter groups include but are not limited to, fluorescein, tetramethyl rhodamine, Texas Red, coumarins, carbonic anhydrase, urease, horseradish peroxidase, dehydrogenases and/or colloidal gold or silver. For radioactive groups, scintillation counting or autoradiographic methods are generally appropriate for detection. Spectroscopic methods may be used to detect dyes, luminescent groups and fluorescent groups. Biotin may be detected using avidin, coupled to a different reporter group (commonly a radioactive or fluorescent group or an enzyme). Enzyme reporter groups may generally be detected by the addition of substrate (generally for a specific period of time), followed by spectroscopic or other analysis of the reaction products.

The present invention also contemplates detecting polynucleotides that encode the tissue-derived glycoproteins of the present invention. Accordingly, detection reagents also include polynucleotides, oligonucleotide primers and probes that specifically detect polynucleotides encoding any of the tissue-derived serum glycoproteins as described herein from any of a variety of tissue sources. Thus, the present invention contemplates detection of expression levels by detection of polynucleotides encoding any of the tissue-derived glycoproteins and tissue-derived serum-glycoproteins described herein using any of a variety of known techniques including, for example, PCR, RT-PCR, quantitative PCR, real-time PCR, northern blot analysis, and the like, as further described herein. Oligonucleotide primers for amplification of the polynucleotides encoding tissue-derived glycoproteins and tissue-derived serum-glycoproteins are within the scope of the present invention where polynucleotide-based detection is desired to better detect tissue-derived serum glycoproteins in a diagnostic assay or kit. Oligonucleotide primers for amplification of the polynucleotides encoding tissue-derived serum glycoproteins are also within the scope of the present invention to amplify transcripts in a biological sample. Many amplification methods are known in the art such as PCR, RT-PCR, quantitative real-time PCR, and the like. The PCR conditions used can be optimized in terms of temperature, annealing times, extension times and number of cycles depending on the oligonucleotide and the polynucleotide to be amplified. Such techniques are well known in the art and are described in, for example, Mullis et al., Cold Spring Harbor Symp. Quant. Biol., 51:263, 1987; Erlich ed., PCR Technology, Stockton Press, NY, 1989. Oligonucleotide primers can be anywhere from 8, 9, 10, 11, 12, 13, 14, 15, 16, 17, 18, 19, 20, 21, 22, 23, 24, 25, 26, 27, 28, 29, or 30 nucleotides in length. In certain embodiments, the oligonucleotide primers/probes of the present invention are typically 35, 40, 45, 50, 55, 60, or more nucleotides in length.

The panels may be comprised of a solid phase surface having attached thereto a plurality of detection reagents each attached at a distinct location. As would be recognized by the skilled artisan, the number of detection reagents on a given panel would be determined from the number of glycoprotein components in a tissue-derived serum glycoprotein set to be measured. In this regard, the plurality of detection reagents may be anywhere from about 2 to about 100, 150, 160, 170, 180, 190, 200 or more detection reagents each specific for a tissue-derived serum glycoprotein. In certain embodiments, the diagnostic panels comprise one or more detection reagents. In another embodiment, a diagnostic panel of the invention may comprise two or more detection reagents. Thus, the diagnostic panels of the invention may comprise a plurality of detection reagents. As would be recognized by the skilled artisan, the number of detection reagents on a given panel would be determined from the number of tissue-derived glycoproteins or glycosites or serum glycoproteins or glycosites to be measured. In this regard, the plurality of detection reagents may be anywhere from 2 to 10, 20, 30, 40, 50, 60, 70, 80, 90, 100, 150, 160, 170, 180, 190, 200 or more detection reagents each specific for a tissue-derived serum glycoprotein or glycosite. In specific embodiments, the panel may comprise for example, 10-50 probes per tissue type and probe two, three, four, five, six, seven, eight, nine, ten, twenty, thirty or more tissues. Accordingly, such arrays/panels may comprise 2500 or more probes.

In one embodiment, the panel comprises at least 3, 4, 5, 6, 7, 8, 9, or 10 detection reagents wherein each reagent specifically bind to or otherwise detects one of the plurality of tissue-derived serum glycoproteins or glycosites that make up a given fingerprint. In another embodiment, the panel comprises at least 11, 12, 13, 14, 15, 16, 17, 18, 19, or 20 detection reagents each specific for one of the plurality of tissue-derived blood glycoproteins that make up a given fingerprint. In a further embodiment, the panel comprises at least 21, 22, 23, 24, 25, 26, 27, 28, 29, or 30 detection reagents each specific for one of the plurality of tissue-derived blood glycoproteins that make up a given fingerprint. In an additional embodiment, the panel comprises at least 31, 32, 33, 34, 35, 36, 37, 38, 39, or 40 detection reagents each specific for one of the plurality of tissue-derived blood glycoproteins that make up a given fingerprint. In yet a further embodiment, the panel comprises at least 41, 42, 43, 44, 45, 46, 47, 48, 49, or 50 detection reagents each specific for one of the plurality of tissue-derived blood glycoproteins that make up a given fingerprint. In an additional embodiment, the panel comprises at least 51, 52, 53, 54, 55, 56, 57, 58, 59, or 60 detection reagents each specific for one of the plurality of tissue-derived blood glycoproteins that make up a given fingerprint. In one embodiment, the panel comprises at least 61, 62, 63, 64, 65, 66, 67, 68, 69, or 70 detection reagents each specific for one of the plurality of tissue-derived blood glycoproteins that make up a given fingerprint. In one embodiment, the panel comprises at least 75, 80, 85, 90, 100, 150, 160, 170, 180, 190, 200, or more, detection reagents each specific for one of the plurality of tissue-derived blood glycoproteins that make up a given fingerprint.

Further in this regard, the solid phase surface may be of any material, including, but not limited to, plastic, polycarbonate, polystyrene, polypropylene, polyethlene, glass, nitrocellulose, dextran, nylon, metal, silicon and carbon nanowires, nanoparticles that can be made of a variety of materials and photolithographic materials. In certain embodiments, the solid phase surface is a chip. In another embodiment, the solid phase surface may comprise microtiter plates, beads, membranes, microparticles, the interior surface of a reaction vessel such as a test tube or other reaction vessel. In other embodiments the peptides will be fractionated by one or more one-dimensional columns using size separations, ion exchange or hydrophobicity properties and, for example, deposited in a MALDI 96 or 384 well plate and then injected into an appropriate mass spectrometer.

In one embodiment, the panel is an addressable array. As such, the addressable array may comprise a plurality of distinct detection reagents, such as antibodies or aptamers, attached to precise locations on a solid phase surface, such as a plastic chip. The position of each distinct detection reagent on the surface is known and therefore “addressable”. In one embodiment, the detection reagents are distinct antibodies that each have specific affinity for one of a plurality of tissue-derived glycopolypeptides or glycosites.

In one embodiment, the detection reagents, such as antibodies, are covalently linked to the solid surface, such as a plastic chip, for example, through the Fc domains of antibodies. In another embodiment, antibodies are adsorbed onto the solid surface. In a further embodiment, the detection reagent, such as an antibody, is chemically conjugated to the solid surface. In a further embodiment, the detection reagents are attached to the solid surface via a linker. In certain embodiments, detection with multiple specific detection reagents is carried out in solution.

Methods of constructing protein arrays, including antibody arrays, are known in the art (see, e.g., U.S. Pat. No. 5,489,678; U.S. Pat. No. 5,252,743; Blawas and Reichert, 1998, Biomaterials 19:595-609; Firestone et al., 1996, J. Amer. Chem. Soc. 18, 9033-9041; Mooney et al., 1996, Proc. Natl. Acad. Sci. 93, 12287-12291; Pirrung et al, 1996, Bioconjugate Chem. 7, 317-321; Gao et al, 1995, Biosensors Bioelectron 10, 317-328; Schena et al, 1995, Science 270, 467-470; Lom et al., 1993, J. Neurosci. Methods, 385-397; Pope et al., 1993, Bioconjugate Chem. 4, 116-171; Schramm et al., 1992, Anal. Biochem. 205, 47-56; Gombotz et al., 1991, J. Biomed. Mater. Res. 25, 1547-1562; Alarie et al., 1990, Analy. Chim. Acta 229, 169-176; Owaku et al, 1993, Sensors Actuators B, 13-14, 723-724; Bhatia et al., 1989, Analy. Biochem. 178, 408-413; Lin et al., 1988, IEEE Trans. Biomed. Engng., 35(6), 466-471).

In one embodiment, the detection reagents, such as antibodies, are arrayed on a chip comprised of electronically activated copolymers of a conductive polymer and the detection reagent. Such arrays are known in the art (see e.g., U.S. Pat. No. 5,837,859 issued Nov. 17, 1998; PCT publication WO 94/22889 dated Oct. 13, 1994). The arrayed pattern may be computer generated and stored. The chips may be prepared in advance and stored appropriately. The antibody array chips can be regenerated and used repeatedly.

The present invention can employ solid substrates, including arrays in some preferred embodiments. Methods and techniques applicable to polymer (including protein) array synthesis have been described in U.S. Ser. No. 09/536,841, WO 00/58516, U.S. Pat. Nos. 5,143,854, 5,242,974, 5,252,743, 5,324,633, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846 and 6,428,752, in PCT Applications Nos. PCT/US99/00730 (International Publication No. WO 99/36760) and PCT/US01/04285 (International Publication No. WO 01/58593), which are all incorporated herein by reference in their entirety for all purposes. Patents that describe synthesis techniques in specific embodiments include U.S. Pat. Nos. 5,412,087, 6,147,205, 6,262,216, 6,310,189, 5,889,165, and 5,959,098.

Nucleic acid arrays that are useful in the present invention include those known in the art and that can be manufactured using the cognate sequences to those nucleic acid sequences set forth in Table 1 and the attached sequence listing, as well as those that are commercially available from Affymetrix (Santa Clara, Calif.) under the brand name GeneChip™. Example arrays are shown on the website at affymetrix dot com. Further exemplary methods of manufacturing and using arrays are provided in, for example, U.S. Pat. Nos. 7,028,629; 7,011,949; 7,011,945; 6,936,419; 6,927,032; 6,924,103; 6,921,642; and 6,818,394 to name a few.

The present invention as related to arrays and microarrays also contemplates many uses for polymers attached to solid substrates. These uses include gene expression monitoring, profiling, library screening, genotyping and diagnostics. Gene expression monitoring and profiling methods and methods useful for gene expression monitoring and profiling are shown in U.S. Pat. Nos. 5,800,992, 6,013,449, 6,020,135, 6,033,860, 6,040,138, 6,177,248 and 6,309,822. Genotyping and uses therefore are shown in U.S. Ser. Nos. 10/442,021, 10/013,598 (U.S. Patent Application Publication 20030036069), and U.S. Pat. Nos. 5,925,525, 6,268,141, 5,856,092, 6,267,152, 6,300,063, 6,525,185, 6,632,611, 5,858,659, 6,284,460, 6,361,947, 6,368,799, 6,673,579 and 6,333,179. Other methods of nucleic acid amplification, labeling and analysis that may be used in combination with the methods disclosed herein are embodied in U.S. Pat. Nos. 5,871,928, 5,902,723, 6,045,996, 5,541,061, and 6,197,506.

In certain embodiments the use of click chemistry (e.g., click reagents) to anchor one or more probes/reagents specific to a glycoprotein as set forth herein or transcript as set forth herein to a detection label or to an array or other surface (e.g., nanoparticle). While such chemistries are well known in the art, in short, the chemistries utilized allow bioconjugation by the formation of triazoles that readily associate with biological targets, through hydrogen bonding and dipole interactions. Chemistries such as this are detailed in the art that is incorporated herein by reference in its entirety and includes Kolb and Sharpless, DDT, Vol. 8 (24), 1128-1137, 2003; U.S. Patent Application Publication No. 20050222427.

In certain embodiments, detection with multiple specific detection reagents is carried out in solution.

The detection reagents of the present invention may be provided in a diagnostic kit. As such a diagnostic kit may comprise any of a variety of appropriate reagents or buffers, enzymes, dyes, colorimetric or other substrates, and appropriate containers to be used in any of a variety of detection assays as described herein. Kits may also comprise one or more positive controls, one or more negative controls, and a protocol for identification of the glycoproteins or glycosites of interest using any one of the assays as described herein.

In certain embodiments of the present invention, kits or panels comprise a plurality of nucleic acid molecules or protein sequences that correspond to two, three, four, five, six, seven, eight, nine, ten, eleven, twelve, thirteen, fourteen, fifteen, sixteen, seventeen, eighteen, nineteen, twenty, or more sequences from Tables 1.

In another embodiment of the present invention, there is an array which comprises a plurality of nucleic acid molecules or protein-binding agents (such as immunoglobulins and antigen-binding fragments thereof) that correspond or specifically bind to two, three, four, five, six, seven, eight, nine, ten, eleven, twelve, thirteen, fourteen, fifteen, sixteen, seventeen, eighteen, nineteen, twenty, or more sequences from Tables 1.

In another embodiment of the present invention, there is a kit for monitoring a course of therapeutic treatment of a disease, comprising a) two gene-specific priming means designed to produce double stranded DNA complementary to a gene selected from the group consisting of any sequence from Table 1; wherein a first priming means contains a sequence which can hybridize to RNA, cDNA or an EST complementary to said gene to create an extension product and a second priming means capable of hybridizing to said extension product; b) an enzyme with reverse transcriptase activity c) an enzyme with thermostable DNA polymerase activity and d) a labeling means; wherein said primers are used to detect the quantitative expression levels of said gene in a test subject.

In another embodiment of the present invention, there is a kit for monitoring progression or regression of a disease, comprising: a) two gene-specific priming means designed to produce double stranded DNA complementary to a gene selected from the group consisting of any sequence in Table 1; wherein a first priming means contains a sequence which can hybridize to RNA, cDNA or an EST complementary to said gene to create an extension product and a second priming means capable of hybridizing to said extension product; b) an enzyme with reverse transcriptase activity c) an enzyme with thermostable DNA polymerase activity and d) a labeling means; wherein said primers are used to detect the quantitative expression levels of said gene in a test subject.

In another embodiment of the present invention, there is a diagnostic panel or kit that comprises a plurality of nucleic acid molecules or polypeptide molecules that identify or correspond to two or more sequences from Table 1.

It would be readily understood by review of the instant specification that while some methods are described as gene or nucleic acid based or polypeptide based, that all such methods would be readily interchangeable. Accordingly, where a method is described that could use a polypeptide for detection of another polypeptide in place of nucleic acid to nucleic acid detection and vice versa, such interchangeability is explicitly considered to be a part of the invention described herein. Likewise, wherein blood is described as the prototypic biological component for analysis, it should be understood that any cell sample, tissue sample, or biological fluid sample may be used interchangeably therewith.

As noted elsewhere herein, perturbation of a normal fingerprint can indicate primary disease of the tissue being tested or secondary, indirect affects on that tissue resulting from disease of another tissue. Perturbation from normal may also include the presence of a glycoprotein in a sample of a patient being tested for a perturbed state not present in a given tissue-derived serum glycoprotein set (e.g., when analyzing a certain patient sample such as in the prostate a glycoprotein or transcript not found in the normal prostate set may appear in a perturbed sample) may be an indicator of disease. Further, the absence of a protein or transcript found in the normal tissue-derived serum glycoprotein set may also be an indicator of a perturbed state.

The levels and locations of tissue-derived serum glycoproteins may change as the result of disease. Thus, in certain embodiments, in vivo imaging techniques can be used to visualize the levels and locations of tissue-derived and/or serum-derived glycoproteins or glycosites in bodily fluid. In this embodiment, exemplary in vivo imaging techniques include, but are not limited to PET, SPECT (Sharma et al; Journal of Magnetic Resonance Imaging (2002), 16: 336-351), MALDI (Stoeckli, et al. Nature Medicine (2001) 7: 493-496), and Fluorescence resonance energy transfer (FRET) (Seker et al, The Journal of Cell Biology, 160 5, (2003) 629-633).

Using the methods described herein, a vast array of tissue-derived glycoprotein blood fingerprints can be defined for any of a variety of diseases as described further herein. As such, the present invention further provides information databases comprising data that make up tissue-derived glycoprotein blood fingerprints as described herein. As such, the databases may comprise the defined differential expression levels as determined using any of a variety of methods such as those described herein, of each of the plurality of tissue-derived glycoproteins that make up a given fingerprint in any of a variety of settings (e.g., normal or disease-associated fingerprints).

Antibodies/Binding Oligopeptides/Binding Organic Molecules

The present invention provides anti-tissue-derived glycoprotein or glycosite specific antibodies and anti-tissue-derived serum glycoprotein or glycosite specific antibodies which may find use herein as therapeutic, diagnostic, and/or imaging agents. Exemplary antibodies include polyclonal, monoclonal, humanized, bispecific, and heteroconjugate antibodies.

Thus, the invention provides antibodies which bind, preferably specifically, to any of the polypeptides described herein. Optionally, the antibody is a monoclonal antibody, antigen-binding fragment thereof, chimeric antibody, humanized antibody, single-chain antibody or antibody that competitively inhibits the binding of an anti-tissue- and/or serum-derived glycopolypeptide antibody to its respective antigenic epitope. Antibodies of the present invention may optionally be conjugated to a growth inhibitory agent or cytotoxic agent such as a toxin, including, for example, a maytansinoid or calicheamicin, an antibiotic, a radioactive isotope, a nucleolytic enzyme, or the like. The antibodies of the present invention may optionally be produced in CHO cells or bacterial cells and preferably induce death of a cell to which they bind. For diagnostic purposes, the antibodies of the present invention may be detectably labeled, attached to a solid support, or the like.

Antibodies may be prepared by any of a variety of techniques known to those of ordinary skill in the art. See, e.g., Harlow and Lane, Antibodies: A Laboratory Manual, Cold Spring Harbor Laboratory, 1988. In general, antibodies can be produced by cell culture techniques, including the generation of monoclonal antibodies using well-established techniques known to the skilled artisan or via transfection of antibody genes into suitable bacterial or mammalian cell hosts, in order to allow for the production of recombinant antibodies. In one technique, an immunogen comprising the polypeptide is initially injected into any of a wide variety of mammals (e.g., mice, rats, rabbits, sheep or goats). In this step, the polypeptides of this invention may serve as the immunogen without modification. Alternatively, particularly for relatively short polypeptides, a superior immune response may be elicited if the polypeptide is joined to a carrier protein, such as bovine serum albumin or keyhole limpet hemocyanin. The immunogen is injected into the animal host, usually according to a predetermined schedule incorporating one or more booster immunizations, and the animals are bled periodically. Polyclonal antibodies specific for the polypeptide may then be purified from such antisera by, for example, affinity chromatography using the polypeptide coupled to a suitable solid support.

In one embodiment, multiple target proteins or peptides are used in a single immune response to generate multiple useful detection reagents simultaneously. In one embodiment, the individual specificities are later separated out.

In certain embodiments, antibody can be generated by phage display methods (such as described by Vaughan, T. J., et al., Nat Biotechnol, 14: 309-314, 1996; and Knappik, A., et al., Mol Biol, 296: 57-86, 2000); ribosomal display (such as described in Hanes, J., et al., Nat Biotechnol, 18: 1287-1292, 2000), or periplasmic expression in E. coli (see e.g., Chen, G., et al., Nat Biotechnol, 19: 537-542, 2001.). In further embodiments, antibodies can be isolated using a yeast surface display library. See e.g., nonimmune library of 10⁹ human antibody scFv fragments as constructed by Feldhaus, M. J., et al., Nat Biotechnol, 21: 163-170, 2003. There are several advantages of this yeast surface display compared to more traditional large nonimmune human antibody repertoires such as phage display, ribosomal display, and periplasmic expression in E. coli 1). The yeast library can be amplified 10¹⁰-fold without measurable loss of clonal diversity and repertoire bias as the expression is under control of the tightly GAL1/10 promoter and expansion can be done under non induction conditions; 2) nanomolar-affinity scFvs can be routinely obtained by magnetic bead screening and flow-cytometric sorting, thus greatly simplified the protocol and capacity of antibody screening; 3) with equilibrium screening, a minimal affinity threshold of the antibodies desired can be set; 4) the binding properties of the antibodies can be quantified directly on the yeast surface; 5) multiplex library screening against multiple antigens simultaneously is possible; and 6) for applications demanding picomolar affinity (e.g. in early diagnosis), subsequent rapid affinity maturation (Kieke, M. C., et al., J Mol Biol, 307: 1305-1315, 2001.) can be carried out directly on yeast clones without further re-cloning and manipulations.

A number of diagnostically useful molecules are known in the art which comprise antigen-binding sites that are capable of exhibiting immunological binding properties of an antibody molecule. The proteolytic enzyme papain preferentially cleaves IgG molecules to yield several fragments, two of which (the F(ab) fragments) each comprise a covalent heterodimer that includes an intact antigen-binding site. The enzyme pepsin is able to cleave IgG molecules to provide several fragments, including the F(ab″)₂ fragment which comprises both antigen-binding sites. An Fv fragment can be produced by preferential proteolytic cleavage of an IgM, and on rare occasions IgG or IgA immunoglobulin molecule. Fv fragments are, however, more commonly derived using recombinant techniques known in the art. The Fv fragment includes a non-covalent V_{H}::V_{L} heterodimer including an antigen-binding site which retains much of the antigen recognition and binding capabilities of the native antibody molecule. Inbar et al. (1972) Proc. Nat. Acad. Sci. USA 69:2659-2662; Hochman et al. (1976) Biochem 15:2706-2710; and Ehrlich et al. (1980) Biochem 19:4091-4096.

A single chain Fv (sFv) polypeptide is a covalently linked V_{H}::V_{L} heterodimer which is expressed from a gene fusion including V_{H}- and V_{L}-encoding genes linked by a peptide-encoding linker. Huston et al. (1988) Proc. Nat. Acad. Sci. USA 85(16):5879-5883. A number of methods have been described to discern chemical structures for converting the naturally aggregated but chemically separated light and heavy polypeptide chains from an antibody V region into an sFv molecule which will fold into a three dimensional structure substantially similar to the structure of an antigen-binding site. See, e.g., U.S. Pat. Nos. 5,091,513 and 5,132,405, to Huston et al.; and U.S. Pat. No. 4,946,778, to Ladner et al.

Each of the above-described molecules includes a heavy chain and a light chain CDR set, respectively interposed between a heavy chain and a light chain FR set which provide support to the CDRS and define the spatial relationship of the CDRs relative to each other. As used herein, the term CDR set refers to the three hypervariable regions of a heavy or light chain V region. Proceeding from the N-terminus of a heavy or light chain, these regions are denoted as CDR1, CDR2, and CDR3 respectively. An antigen-binding site, therefore, includes six CDRs, comprising the CDR set from each of a heavy and a light chain V region. A polypeptide comprising a single CDR, (e.g., a CDR1, CDR2 or CDR3) is referred to herein as a molecular recognition unit. Crystallographic analysis of a number of antigen-antibody complexes has demonstrated that the amino acid residues of CDRs form extensive contact with bound antigen, wherein the most extensive antigen contact is with the heavy chain CDR3. Thus, the molecular recognition units are primarily responsible for the specificity of an antigen-binding site.

As used herein, the term FR set refers to the four flanking amino acid sequences which frame the CDRs of a CDR set of a heavy or light chain V region. Some FR residues may contact bound antigen; however, FRs are primarily responsible for folding the V region into the antigen-binding site, particularly the FR residues directly adjacent to the CDRS. Within FRs, certain amino residues and certain structural features are very highly conserved. In this regard, all V region sequences contain an internal disulfide loop of around 90 amino acid residues. When the V regions fold into a binding-site, the CDRs are displayed as projecting loop motifs which form an antigen-binding surface. It is generally recognized that there are conserved structural regions of FRs which influence the folded shape of the CDR loops into certain canonical structures regardless of the precise CDR amino acid sequence. Further, certain FR residues are known to participate in non-covalent interdomain contacts which stabilize the interaction of the antibody heavy and light chains.

In other embodiments of the present invention, the invention provides vectors comprising DNA encoding any of the herein described antibodies. Host cell comprising any such vector are also provided. By way of example, the host cells may be CHO cells, E. coli cells, or yeast cells. A process for producing any of the herein described antibodies is further provided and comprises culturing host cells under conditions suitable for expression of the desired antibody and recovering the desired antibody from the cell culture.

1. Polyclonal Antibodies

Polyclonal antibodies are preferably raised in animals by multiple subcutaneous (sc) or intraperitoneal (ip) injections of the relevant antigen and an adjuvant. It may be useful to conjugate the relevant antigen (especially when synthetic peptides are used) to a protein that is immunogenic in the species to be immunized. For example, the antigen can be conjugated to keyhole limpet hemocyanin (KLH), serum albumin, bovine thyroglobulin, or soybean trypsin inhibitor, using a bifunctional or derivatizing agent, e.g., maleimidobenzoyl sulfosuccinimide ester (conjugation through cysteine residues), N-hydroxysuccinimide (through lysine residues), glutaraldehyde, succinic anhydride, SOCl₂, or R¹N═C′NR, where R and R¹ are different alkyl groups.

Animals are immunized against the antigen, immunogenic conjugates, or derivatives by combining, e.g., 100 μg or 5 μg of the protein or conjugate (for rabbits or mice, respectively) with 3 volumes of Freund's complete adjuvant and injecting the solution intradermally at multiple sites. One month later, the animals are boosted with ⅕ to 1/10 the original amount of peptide or conjugate in Freund's complete adjuvant by subcutaneous injection at multiple sites. Seven to 14 days later, the animals are bled and the serum is assayed for antibody titer. Animals are boosted until the titer plateaus. Conjugates also can be made in recombinant cell culture as protein fusions. Also, aggregating agents such as alum are suitably used to enhance the immune response.

2. Monoclonal Antibodies

Monoclonal antibodies may be made using the hybridoma method first described by Kohler et al., Nature, 256:495 (1975), or may be made by recombinant DNA methods (U.S. Pat. No. 4,816,567).

In the hybridoma method, a mouse or other appropriate host animal, such as a hamster, is immunized as described above to elicit lymphocytes that produce or are capable of producing antibodies that will specifically bind to the protein used for immunization. Alternatively, lymphocytes may be immunized in vitro. After immunization, lymphocytes are isolated and then fused with a myeloma cell line using a suitable fusing agent, such as polyethylene glycol, to form a hybridoma cell (Goding, Monoclonal Antibodies: Principles and Practice, pp. 59-103 (Academic Press, 1986)).

The hybridoma cells thus prepared are seeded and grown in a suitable culture medium which medium preferably contains one or more substances that inhibit the growth or survival of the unfused, parental myeloma cells (also referred to as fusion partner). For example, if the parental myeloma cells lack the enzyme hypoxanthine guanine phosphoribosyl transferase (HGPRT or HPRT), the selective culture medium for the hybridomas typically will include hypoxanthine, aminopterin, and thymidine (HAT medium), which substances prevent the growth of HGPRT-deficient cells.

Preferred fusion partner myelomacells are those that fuse efficiently, support stable high-level production of antibody by the selected antibody-producing cells, and are sensitive to a selective medium that selects against the unfused parental cells. Preferred myeloma cell lines are murine myeloma lines, such as those derived from MOPC-21 and MPC-11 mouse tumors available from the Salk Institute Cell Distribution Center, San Diego, Calif. USA, and SP-2 and derivatives e.g., X63-Ag8-653 cells available from the American Type Culture Collection, Manassas, Va., USA. Human myeloma and mouse-human heteromyeloma cell lines also have been described for the production of human monoclonal antibodies (Kozbor, J. Immunol., 133:3001 (1984); and Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunosorbent assay (ELISA).

The binding affinity of the monoclonal antibody can, for example, be determined by the Scatchard analysis described in Munson et al., Anal. Biochem., 107:220 (1980).

Once hybridoma cells that produce antibodies of the desired specificity, affinity, and/or activity are identified, the clones may be subcloned by limiting dilution procedures and grown by standard methods (Goding, Monoclonal Antibodies: Principles and Practice, pp. 59-103 (Academic Press, 1986)). Suitable culture media for this purpose include, for example, D-MEM or RPMI-1640 medium. In addition, the hybridoma cells may be grown in vivo as ascites tumors in an animal e.g., by i.p. injection of the cells into mice.

The monoclonal antibodies secreted by the subclones are suitably separated from the culture medium, ascites fluid, or serum by conventional antibody purification procedures such as, for example, affinity chromatography (e.g., using protein A or protein G-Sepharose) or ion-exchange chromatography, hydroxylapatite chromatography, gel electrophoresis, dialysis, etc.

DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as E. coli cells, simian COS cells, Chinese Hamster Ovary (CHO) cells, or myeloma cells that do not otherwise produce antibody protein, to obtain the synthesis of monoclonal antibodies in the recombinant host cells. Review articles on recombinant expression in bacteria of DNA encoding the antibody include Skerra et al., Curr. Opinion in Immunol., 5:256-262 (1993) and Pluckthun, Immunol. Revs. 130:151-188 (1992).

In a further embodiment, monoclonal antibodies or antigen-binding fragments thereof can be isolated from antibody phage libraries generated using the techniques described in McCafferty et al., Nature, 348:552-554 (1990). Clackson et al., Nature, 352:624-628 (1991) and Marks et al., J. Mol. Biol., 222:581-597 (1991) describe the isolation of murine and human antibodies, respectively, using phage libraries. Subsequent publications describe the production of high affinity (nM range) human antibodies by chain shuffling (Marks et al., Bio/Technology, 10:779-783 (1992)), as well as combinatorial infection and in vivo recombination as a strategy for constructing very large phage libraries (Waterhouse et al., Nuc. Acids. Res. 21:2265-2266 (1993)). Thus, these techniques are viable alternatives to traditional monoclonal antibody hybridoma techniques for isolation of monoclonal antibodies.

The DNA that encodes the antibody may be modified to produce chimeric or fusion antibody polypeptides, for example, by substituting human heavy chain and light chain constant domain (C.sub.H and C.sub.L) sequences for the homologous murine sequences (U.S. Pat. No. 4,816,567; and Morrison, et al., Proc. Natl Acad. Sci. USA, 81:6851 (1984)), or by fusing the immunoglobulin coding sequence with all or part of the coding sequence for a non-immunoglobulin polypeptide (heterologous polypeptide). The non-immunoglobulin polypeptide sequences can substitute for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

3. Human and Humanized Antibodies

The anti-tissue-and/or serum-derived glycoprotein or glycosite antibodies of the invention may further comprise humanized antibodies or human antibodies. Humanized forms of non-human (e.g., murine) antibodies are chimeric immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers [Jones et al., Nature, 321:522-525 (1986); Riechmann et al., Nature, 332:323-327 (1988); Verhoeyen et al. Science, 239:1534-1536 (1988)], by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The choice of human variable domains, both light and heavy, to be used in making the humanized antibodies is very important to reduce antigenicity and HAMA response (human anti-mouse antibody) when the antibody is intended for human therapeutic use. According to the so-called “best-fit” method, the sequence of the variable domain of a rodent antibody is screened against the entire library of known human variable domain sequences. The human V domain sequence which is closest to that of the rodent is identified and the human framework region (FR) within it accepted for the humanized antibody (Sims et al., J. Immunol. 151:2296 (1993); Chothia et al., J. Mol. Biol., 196:901 (1987)). Another method uses a particular framework region derived from the consensus sequence of all human antibodies of a particular subgroup of light or heavy chains. The same framework may be used for several different humanized antibodies (Carter et al., Proc. Natl. Acad. Sci. USA, 89:4285 (1992); Presta et al., J. Immunol. 151:2623 (1993)).

It is further important that antibodies be humanized with retention of high binding affinity for the antigen and other favorable biological properties. To achieve this goal, according to a preferred method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

Various forms of a humanized anti-tissue-/and/or serum-derived glycoprotein or glycosite antibody are contemplated. For example, the humanized antibody may be an antibody fragment, such as a Fab, which is optionally conjugated with one or more cytotoxic agent(s) in order to generate an immunoconjugate. Alternatively, the humanized antibody may be an intact antibody, such as an intact IgG1 antibody.

As an alternative to humanization, human antibodies can be generated. For example, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (J.sub.H) gene in chimeric and germ-line mutant mice results in complete inhibition of endogenous antibody production. Transfer of the human germ-line immunoglobulin gene array into such germ-line mutant mice will result in the production of human antibodies upon antigen challenge. See, e.g., Jakobovits et al., Proc. Natl. Acad. Sci. USA, 90:2551 (1993); Jakobovits et al., Nature, 362:255-258 (1993); Bruggemann et al., Year in Immuno. 7:33 (1993); U.S. Pat. Nos. 5,545,806, 5,569,825, 5,591,669 (all of GenPharm); U.S. Pat. No. 5,545,807; and WO 97/17852.

Alternatively, phage display technology (McCafferty et al., Nature 348:552-553) can be used to produce human antibodies and antigen-binding fragments thereof in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B-cell. Phage display can be performed in a variety of formats, reviewed in, e.g., Johnson, Kevin S. and Chiswell, David J., Current Opinion in Structural Biology 3:564-571 (1993). Several sources of V-gene segments can be used for phage display. Clackson et al., Nature, 352:624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of probes (including self-antigens) can be isolated essentially following the techniques described by Marks et al., J. Mol. Biol. 222:581-597 (1991), or Griffith et al., EMBO J. 12:725-734 (1993). See, also, U.S. Pat. Nos. 5,565,332 and 5,573,905.

As discussed above, human antibodies may also be generated by in vitro activated B cells (see U.S. Pat. Nos. 5,567,610 and 5,229,275).

4. Antigen-Binding Antibody Fragments

In certain circumstances there are advantages of using antibody fragments, rather than whole antibodies. The smaller size of the fragments allows for rapid clearance, and may lead to improved access to solid tumors.

Various techniques have been developed for the production of antibody fragments. Traditionally, these fragments were derived via proteolytic digestion of intact antibodies (see, e.g., Morimoto et al., Journal of Biochemical and Biophysical Methods 24:107-117 (1992); and Brennan et al., Science, 229:81 (1985)). However, these fragments can now be produced directly by recombinant host cells. Fab, Fv and ScFv antibody fragments can all be expressed in and secreted from E. coli, thus allowing the facile production of large amounts of these fragments. Antibody fragments can be isolated from the antibody phage libraries discussed above. Alternatively, Fab′-SH fragments can be directly recovered from E. coli and chemically coupled to form F(ab′)₂ fragments (Carter et al., Bio/Technology 10:163-167 (1992)). According to another approach, F(ab′)₂ fragments can be isolated directly from recombinant host cell culture. Fab and F(ab′)₂ fragment with increased in vivo half-life comprising a salvage receptor binding epitope residues are described in U.S. Pat. No. 5,869,046. Other techniques for the production of antibody fragments will be apparent to the skilled practitioner. In other embodiments, the antibody of choice is a single chain Fv fragment (scFv). See WO 93/16185; U.S. Pat. No. 5,571,894; and U.S. Pat. No. 5,587,458. Fv and sFv are the only species with intact combining sites that are devoid of constant regions; thus, they are suitable for reduced nonspecific binding during in vivo use. sFv fusion proteins may be constructed to yield fusion of an effector protein at either the amino or the carboxy terminus of an sFv. See Antibody Engineering, ed. Borrebaeck, supra. The antibody fragment may also be a “linear antibody”, e.g., as described in U.S. Pat. No. 5,641,870 for example. Such linear antibody fragments may be monospecific or bispecific.

5. Bispecific Antibodies

Bispecific antibodies are antibodies that have binding specificities for at least two different epitopes. Exemplary bispecific antibodies may bind to two different epitopes of an glycoprotein as described herein. Other such antibodies may combine a tissue-derived or serum derived glycoprotein binding site with a binding site for another protein. Alternatively, an anti-tissue-and/or serum-derived arm may be combined with an arm which binds to a triggering molecule on a leukocyte such as a T-cell receptor molecule (e.g. CD3), or Fc receptors for IgG (FcγR), such as FcγRI (CD64), FcγRII (CD32) and FcγRIII (CD16), so as to focus and localize cellular defense mechanisms to the cell expressing a glycoprotein of interest. Bispecific antibodies may also be used for diagnostic purposes, attaching imaging agents or localizing cytotoxic agents to cells which express glycoproteins of interest. These antibodies possess an arm that binds to the glycoprotein or glycosite of interest and an arm which binds the cytotoxic agent (e.g., saporin, anti-interferon-.alpha., vinca alkaloid, ricin A chain, methotrexate or radioactive isotope hapten). Bispecific antibodies can be prepared as full length antibodies or antibody fragments (e.g., F(ab′)₂ bispecific antibodies).

WO 96/16673 describes a bispecific anti-ErbB2/anti-FcγRIII antibody and U.S. Pat. No. 5,837,234 discloses a bispecific anti-ErbB2/anti-FcγRI antibody. A bispecific anti-ErbB2/Fc .alpha. antibody is shown in WO98/02463. U.S. Pat. No. 5,821,337 teaches a bispecific anti-ErbB2/anti-CD3 antibody.

Methods for making bispecific antibodies are known in the art. Traditional production of full length bispecific antibodies is based on the co-expression of two immunoglobulin heavy chain-light chain pairs, where the two chains have different specificities (Millstein et al., Nature 305:537-539 (1983)). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829, and in Traunecker et al., EMBO J. 10:3655-3659 (1991).

According to a different approach, antibody variable domains with the desired binding specificities (antibody-antigen combining sites) are fused to immunoglobulin constant domain sequences. Preferably, the fusion is with an Ig heavy chain constant domain, comprising at least part of the hinge, C_{H2}, and C_{H3} regions. It is preferred to have the first heavy-chain constant region (C_{H1}) containing the site necessary for light chain bonding, present in at least one of the fusions. DNAs encoding the immunoglobulin heavy chain fusions and, if desired, the immunoglobulin light chain, are inserted into separate expression vectors, and are co-transfected into a suitable host cell. This provides for greater flexibility in adjusting the mutual proportions of the three polypeptide fragments in embodiments when unequal ratios of the three polypeptide chains used in the construction provide the optimum yield of the desired bispecific antibody. It is, however, possible to insert the coding sequences for two or all three polypeptide chains into a single expression vector when the expression of at least two polypeptide chains in equal ratios results in high yields or when the ratios have no significant affect on the yield of the desired chain combination.

In a preferred embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690. For further details of generating bispecific antibodies see, for example, Suresh et al., Methods in Enzymology 121:210 (1986).

According to another approach described in U.S. Pat. No. 5,731,168, the interface between a pair of antibody molecules can be engineered to maximize the percentage of heterodimers which are recovered from recombinant cell culture. The preferred interface comprises at least a part of the C.sub.H3 domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373, and EP 03089). Heteroconjugate antibodies may be made using any convenient cross-linking methods. Suitable cross-linking agents are well known in the art, and are disclosed in U.S. Pat. No. 4,676,980, along with a number of cross-linking techniques.

Techniques for generating bispecific antibodies from antibody fragments have also been described in the literature. For example, bispecific antibodies can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol complexing agent, sodium arsenite, to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Recent progress has facilitated the direct recovery of Fab′-SH fragments from E. coli, which can be chemically coupled to form bispecific antibodies. Shalaby et al., J. Exp. Med. 175: 217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets. Various techniques for making and isolating bispecific antibody fragments directly from recombinant cell culture have also been described. For example, bispecific antibodies have been produced using leucine zippers. Kostelny et al., J. Immunol. 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a V.sub.H connected to a V.sub.L by a linker which is too short to allow pairing between the two domains on the same chain. Accordingly, the V.sub.H and V.sub.L domains of one fragment are forced to pair with the complementary V.sub.L and V.sub.H domains of another fragment, thereby forming two antigen-binding sites. Another strategy for making bispecific antibody fragments by the use of single-chain Fv (sFv) dimers has also been reported. See Gruber et al., J. Immunol., 152:5368 (1994).

Antibodies with more than two valencies are contemplated. For example, trispecific antibodies can be prepared. Tutt et al., J. Immunol. 147:60 (1991).

6. Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells [U.S. Pat. No. 4,676,980], and for treatment of HIV infection [WO 91/00360; WO 92/200373; EP 03089]. It is contemplated that the antibodies may be prepared in vitro using known methods in synthetic protein chemistry, including those involving crosslinking agents. For example, immunotoxins may be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

7. Multivalent Antibodies

A multivalent antibody may be internalized (and/or catabolized) faster than a bivalent antibody by a cell expressing an antigen to which the antibodies bind. The antibodies of the present invention can be multivalent antibodies (which are other than of the IgM class) with three or more antigen binding sites (e.g. tetravalent antibodies), which can be readily produced by recombinant expression of nucleic acid encoding the polypeptide chains of the antibody. The multivalent antibody can comprise a dimerization domain and three or more antigen binding sites. The preferred dimerization domain comprises (or consists of) an Fc region or a hinge region. In this scenario, the antibody will comprise an Fc region and three or more antigen binding sites amino-terminal to the Fc region. The preferred multivalent antibody herein comprises (or consists of) three to about eight, but preferably four, antigen binding sites. The multivalent antibody comprises at least one polypeptide chain (and preferably two polypeptide chains), wherein the polypeptide chain(s) comprise two or more variable domains. For instance, the polypeptide chain(s) may comprise VD1-(X1).sub.n-VD2-(X2).sub.n-Fc, wherein VD1 is a first variable domain, VD2 is a second variable domain, Fc is one polypeptide chain of an Fc region, X1 and X2 represent an amino acid or polypeptide, and n is 0 or 1. For instance, the polypeptide chain(s) may comprise: VH-CH1-flexible linker-VH-CH1-Fc region chain; or VH-CH1-VH-CH1-Fc region chain. The multivalent antibody herein preferably further comprises at least two (and preferably four) light chain variable domain polypeptides. The multivalent antibody herein may, for instance, comprise from about two to about eight light chain variable domain polypeptides. The light chain variable domain polypeptides contemplated here comprise a light chain variable domain and, optionally, further comprise a CL domain.

8. Effector Function Engineering

It may be desirable to modify the antibody of the invention with respect to effector function, e.g., so as to enhance antigen-dependent cell-mediated cyotoxicity (ADCC) and/or complement dependent cytotoxicity (CDC) of the antibody. This may be achieved by introducing one or more amino acid substitutions in an Fc region of the antibody. Alternatively or additionally, cysteine residue(s) may be introduced in the Fc region, thereby allowing interchain disulfide bond formation in this region. The homodimeric antibody thus generated may have improved internalization capability and/or increased complement-mediated cell killing and antibody-dependent cellular cytotoxicity (ADCC). See Caron et al., J. Exp Med. 176:1191-1195 (1992) and Shopes, B. J. Immunol. 148:2918-2922 (1992). Homodimeric antibodies with enhanced anti-tumor activity may also be prepared using heterobifunctional cross-linkers as described in Wolff et al., Cancer Research 53:2560-2565 (1993). Alternatively, an antibody can be engineered which has dual Fc regions and may thereby have enhanced complement lysis and ADCC capabilities. See Stevenson et al., Anti-Cancer Drug Design 3:219-230 (1989). To increase the serum half life of the antibody, one may incorporate a salvage receptor binding epitope into the antibody (especially an antibody fragment) as described in U.S. Pat. No. 5,739,277, for example. As used herein, the term “salvage receptor binding epitope” refers to an epitope of the Fc region of an IgG molecule (e.g., IgG.sub.1, IgG₂, IgG.sub.3, or IgG.sub.4) that is responsible for increasing the in vivo serum half-life of the IgG molecule.

9. Immunoconjugate

The invention also pertains to immunoconjugates comprising an antibody conjugated to a cytotoxic agent such as a chemotherapeutic agent, a growth inhibitory agent, a toxin (e.g., an enzymatically active toxin of bacterial, fungal, plant, or animal origin, or fragments thereof), or a radioactive isotope (i.e., a radioconjugate).

Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²12Bi, ¹³¹I, ¹³¹In, ⁹⁰Y, and ¹⁸⁶Re. Conjugates of the antibody and cytotoxic agent are made using a variety of bifunctional protein-coupling agents such as N-succinimidyl-3-(2-pyridyldithiol)propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis(p-azidobenzoyl)hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyidiethylene triaminepentaacetic acid (MX-DTPA) is an exemplary chelating agent for conjugation of radionucleotide to the antibody. See WO94/11026.

Conjugates of an antibody and one or more small molecule toxins, such as a calicheamicin, maytansinoids, a trichothene, and CC1065, and the derivatives of these toxins that have toxin activity, are also contemplated herein.

10. Immunoliposomes

The antibodies disclosed herein may also be formulated as immunoliposomes. A “liposome” is a small vesicle composed of various types of lipids, phospholipids and/or surfactant which is useful for delivery of a drug to a mammal. The components of the liposome are commonly arranged in a bilayer formation, similar to the lipid arrangement of biological membranes. Liposomes containing the antibody are prepared by methods known in the art, such as described in Epstein et al., Proc. Natl. Acad. Sci. USA 82:3688 (1985); Hwang et al., Proc. Natl. Acad. Sci. USA 77:4030 (1980); U.S. Pat. Nos. 4,485,045 and 4,544,545; and WO97/38731 published Oct. 23, 1997. Liposomes with enhanced circulation time are disclosed in U.S. Pat. No. 5,013,556.

Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al., J. Biol. Chem. 257:286-288 (1982) via a disulfide interchange reaction. A chemotherapeutic agent is optionally contained within the liposome. See Gabizon et al., J. National Cancer Inst. 81(19):1484 (1989).

In another embodiment, the invention provides oligopeptides which bind, preferably specifically, to any of the tissue-derived glycoproteins, glycopeptide or glycosites described herein. Optionally, the oligopeptides of the present invention may be conjugated to a growth inhibitory agent or cytotoxic agent such as a toxin, including, for example, a maytansinoid or calicheamicin, an antibiotic, a radioactive isotope, a nucleolytic enzyme, or the like. The oligopeptides of the present invention may optionally be produced in CHO cells or bacterial cells and preferably induce death of a cell to which they bind. For diagnostic purposes, the binding oligopeptides of the present invention may be detectably labeled, attached to a solid support, or the like.

Binding oligopeptides of the present invention are oligopeptides that bind, preferably specifically, to tissue-derived glycoproteins or glycosites and serum glycoproteins thereof as described herein (see Table 1). Binding oligopeptides may be chemically synthesized using known oligopeptide synthesis methodology or may be prepared and purified using recombinant technology. Binding oligopeptides are usually at least about 5 amino acids in length, alternatively at least about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 amino acids in length or more, wherein such oligopeptides that are capable of binding, preferably specifically, to glycopolypeptide or glycosite as described herein. Binding oligopeptides may be identified without undue experimentation using well known techniques. In this regard, it is noted that techniques for screening oligopeptide libraries for oligopeptides that are capable of specifically binding to a polypeptide target are well known in the art (see, e.g., U.S. Pat. Nos. 5,556,762, 5,750,373, 4,708,871, 4,833,092, 5,223,409, 5,403,484, 5,571,689, 5,663,143; PCT Publication Nos. WO 84/03506 and WO084/03564; Geysen et al., Proc. Natl. Acad. Sci. U.S.A., 81:3998-4002 (1984); Geysen et al., Proc. Natl. Acad. Sci. U.S.A., 82:178-182 (1985); Geysen et al., in Synthetic Peptides as Antigens, 130-149 (1986); Geysen et al., J. Immunol. Meth., 102:259-274 (1987); Schoofs et al., J. Immunol., 140:611-616 (1988), Cwirla, S. E. et al. (1990) Proc. Natl. Acad. Sci. USA, 87:6378; Lowman, H. B. et al. (1991) Biochemistry, 30:10832; Clackson, T. et al. (1991) Nature, 352: 624; Marks, J. D. et al. (1991), J. Mol. Biol., 222:581; Kang, A. S. et al. (1991) Proc. Natl. Acad. Sci. USA, 88:8363, and Smith, G. P. (1991) Current Opin. Biotechnol., 2:668).

In this regard, bacteriophage (phage) display is one well known technique which allows one to screen large oligopeptide libraries to identify member(s) of those libraries which are capable of specifically binding to a polypeptide target. Phage display is a technique by which variant polypeptides are displayed as fusion proteins to the coat protein on the surface of bacteriophage particles (Scott, J. K. and Smith, G. P. (1990) Science 249: 386). The utility of phage display lies in the fact that large libraries of selectively randomized protein variants (or randomly cloned cDNAs) can be rapidly and efficiently sorted for those sequences that bind to a target molecule with high affinity. Display of peptide (Cwirla, S. E. et al. (1990) Proc. Natl. Acad. Sci. USA, 87:6378) or protein (Lowman, H. B. et al. (1991) Biochemistry, 30:10832; Clackson, T. et al. (1991) Nature, 352: 624; Marks, J. D. et al. (1991), J. Mol. Biol., 222:581; Kang, A. S. et al. (1991) Proc. Natl. Acad. Sci. USA, 88:8363) libraries on phage have been used for screening millions of polypeptides or oligopeptides for ones with specific binding properties (Smith, G. P. (1991) Current Opin. Biotechnol., 2:668). Sorting phage libraries of random mutants requires a strategy for constructing and propagating a large number of variants, a procedure for affinity purification using the target receptor, and a means of evaluating the results of binding enrichments. U.S. Pat. Nos. 5,223,409, 5,403,484, 5,571,689, and 5,663,143.

Although most phage display methods have used filamentous phage, lambdoid phage display systems (WO95/34683; U.S. Pat. No. 5,627,024), T4 phagedisplay systems (Ren, Z-J. et al. (1998) Gene 215:439; Zhu, Z. (1997) CAN 33:534; Jiang, J. et al. (1997) can 128:44380; Ren, Z-J. et al. (1997) CAN 127:215644; Ren, Z-J. (1996) Protein Sci. 5:1833; Efimov, V. P. et al. (1995) Virus Genes 10:173) and T7 phage display systems (Smith, G. P. and Scott, J. K. (1993) Methods in Enzymology, 217, 228-257; U.S. Pat. No. 5,766,905) are also known.

Many other improvements and variations of the basic phage display concept have now been developed. These improvements enhance the ability of display systems to screen peptide libraries for binding to selected target molecules and to display functional proteins with the potential of screening these proteins for desired properties. Combinatorial reaction devices for phage display reactions have been developed (WO 98/14277) and phage display libraries have been used to analyze and control bimolecular interactions (WO 98/20169; WO 98/20159) and properties of constrained helical peptides (WO 98/20036). WO 97/35196 describes a method of isolating an affinity ligand in which a phage display library is contacted with one solution in which the ligand will bind to a target molecule and a second solution in which the affinity ligand will not bind to the target molecule, to selectively isolate binding ligands. WO 97/46251 describes a method of biopanning a random phage display library with an affinity purified antibody and then isolating binding phage, followed by a micropanning process using microplate wells to isolate high affinity binding phage. The use of Staphlylococcus aureus protein A as an affinity tag has also been reported (Li et al. (1998) Mol Biotech., 9:187). WO 97/47314 describes the use of substrate subtraction libraries to distinguish enzyme specificities using a combinatorial library which may be a phage display library. A method for selecting enzymes suitable for use in detergents using phage display is described in WO 97/09446. Additional methods of selecting specific binding proteins are described in U.S. Pat. Nos. 5,498,538, 5,432,018, and WO 98/15833.

Methods of generating peptide libraries and screening these libraries are also disclosed in U.S. Pat. Nos. 5,723,286, 5,432,018, 5,580,717, 5,427,908, 5,498,530, 5,770,434, 5,734,018, 5,698,426, 5,763,192, and 5,723,323.

In other embodiments of the present invention, the invention provides vectors comprising DNA encoding any of the herein described oligopeptides. Host cell comprising any such vector are also provided. By way of example, the host cells may be CHO cells, E. coli cells, or yeast cells. A process for producing any of the herein described oligopeptides is further provided and comprises culturing host cells under conditions suitable for expression of the desired oligopeptide and recovering the desired oligopeptide from the cell culture.

In another embodiment, the invention provides small organic molecules which bind, preferably specifically, to any of the glycoproteins or glycosites described herein and listed in Table 1. Optionally, the organic molecules of the present invention may be conjugated to a growth inhibitory agent or cytotoxic agent such as a toxin, including, for example, a maytansinoid or calicheamicin, an antibiotic, a radioactive isotope, a nucleolytic enzyme, or the like. The binding organic molecules of the present invention preferably induce death of a cell to which they bind. For diagnostic purposes, the binding organic molecules of the present invention may be detectably labeled, attached to a solid support, or the like.

Binding organic molecules of the present invention are organic molecules other than oligopeptides or antibodies as defined herein that bind, preferably specifically, to any of the tissue-derived and tissue-derived serum glycoproteins or glycosites described herein and listed in Table 1. Binding organic molecules may be identified and chemically synthesized using known methodology (see, e.g., PCT Publication Nos. WO00/00823 and WO00/39585). Binding organic molecules are usually less than about 2000 daltons in size, alternatively less than about 1500, 750, 500, 250 or 200 daltons in size, wherein such organic molecules that are capable of binding, preferably specifically, to a glycoprotein or glycosites as described herein may be identified without undue experimentation using well known techniques. In this regard, it is noted that techniques for screening organic molecule libraries for molecules that are capable of binding to a polypeptide target are well known in the art (see, e.g., PCT Publication Nos. WO00/00823 and WO00/39585). Binding organic molecules may be, for example, aldehydes, ketones, oximes, hydrazones, semicarbazones, carbazides, primary amines, secondary amines, tertiary amines, N-substituted hydrazines, hydrazides, alcohols, ethers, thiols, thioethers, disulfides, carboxylic acids, esters, amides, ureas, carbamates, carbonates, ketals, thioketals, acetals, thioacetals, aryl halides, aryl sulfonates, alkyl halides, alkyl sulfonates, aromatic compounds, heterocyclic compounds, anilines, alkenes, alkynes, diols, amino alcohols, oxazolidines, oxazolines, thiazolidines, thiazolines, enamines, sulfonamides, epoxides, aziridines, isocyanates, sulfonyl chlorides, diazo compounds, acid chlorides, or the like.

Nucleic Acid Analysis

As would be recognized by the skilled artisan, the level of a particular glycoprotein can also be determed by detecting the level of expression of the polynucleotide encoding the glycoprotein. Illustrative glycoproteins and glycosites of the invention are set forth in Table 1 and SEQ ID NOs:1-11,375; illustrative polynucleotides encoding these glycoproteins are set forth in Table 1 and SEQ ID NOs:11,376-14,917. Note that the sequences set forth in the sequence listing are identified by mapping the identified glycosite sequence to public sequence databases available as of the time of filing. As the skilled artisan would immediately recognize, the disclosed glycoprotein sequences and the corresponding polynucleotide sequences represent the mapped sequences available in the public databases at the time of mapping and these sequences may change slightly over time as sequences in the databases are corrected/updated. Accordingly, as would be recognized by the skilled artisan, updated/corrected sequences are also contemplated for use herein. Further, isoforms and variants of the disclosed sequences are also contemplated for use in the diagnostic/prognostic panels and methods of the present invention.

Accordingly, in one embodiment of the present invention, the invention provides an isolated nucleic acid molecule having a nucleotide sequence that encodes a tissue-derived target glycopolypeptide or fragment thereof.

In certain aspects, the isolated nucleic acid molecule comprises a nucleotide sequence having at least about 80% nucleic acid sequence identity, alternatively at least about 81%, 82%, 83%, 84%, 85%, 86%, 87%, 88%, 89%, 90%, 91%, 92%, 93%, 94%, 95%, 96%, 97%, 98%, 99% or 100% nucleic acid sequence identity, to (a) a polynucleotide molecule encoding a full-length tissue-derived glycopolypeptide having an amino acid sequence as disclosed herein, a tissue-derived glycopolypeptide amino acid sequence lacking the signal peptide as disclosed herein, an extracellular domain of a transmembrane tissue-derived polypeptide, with or without the signal peptide, as disclosed herein or any other specifically defined fragment of a full-length tissue-derived glycoprotein amino acid sequence as disclosed herein, or (b) the complement of the polynucleotide molecule of (a).

In other aspects, the isolated nucleic acid molecule comprises a nucleotide sequence having at least about 80% nucleic acid sequence identity, alternatively at least about 81%, 82%, 83%, 84%, 85%, 86%, 87%, 88%, 89%, 90%, 91%, 92%, 93%, 94%, 95%, 96%, 97%, 98%, 99% or 100% nucleic acid sequence identity, to (a) a polynucleotide molecule comprising the coding sequence of a full-length tissue-derived glycoprotein cDNA as disclosed herein, the coding sequence of a tissue-derived glycoprotein lacking the signal peptide as disclosed herein, the coding sequence of an extracellular domain of a transmembrane tissue-derived glycoprotein, with or without the signal peptide, as disclosed herein or the coding sequence of any other specifically defined fragment of the full-length tissue-derived glycoprotein amino acid sequence as disclosed herein, or (b) the complement of the polynucleotide molecule of (a).

In further aspects, the invention concerns an isolated nucleic acid molecule comprising a nucleotide sequence having at least about 80% nucleic acid sequence identity, alternatively at least about 81%, 82%, 83%, 84%, 85%, 86%, 87%, 88%, 89%, 90%, 91%, 92%, 93%, 94%, 95%, 96%, 97%, 98%, 99% or 100% nucleic acid sequence identity, to (a) a nucleic acid molecule that encodes the same mature polypeptide encoded by the full-length coding region of any of the human protein cDNAs as disclosed herein, or (b) the complement of the nucleic acid molecule of (a).

In other aspects, the present invention is directed to isolated nucleic acid molecules which hybridize to (a) a nucleotide sequence encoding a tissue-derived glycoprotein having a full-length amino acid sequence as disclosed herein or any other specifically defined fragment of a full-length tissue-derived glycoprotein amino acid sequence as disclosed herein, or (b) the complement of the nucleotide sequence of (a). In this regard, an embodiment of the present invention is directed to fragments of a full-length tissue-derived glycoprotein coding sequence, or the complement thereof, as disclosed herein, that may find use as, for example, hybridization probes useful as, for example, diagnostic probes, antisense oligonucleotide probes, or for encoding fragments of a full-length tissue-derived glycoprotein that may optionally encode a polypeptide comprising a binding site for an anti-tissue-derived glycoprotein antibody, a tissue-derived glycoprotein binding oligopeptide or other small organic molecule that binds to a tissue-derived glycoprotein. Illustrative fragments include the glycosites as listed in Table 1. Such nucleic acid fragments are usually at least about 5 nucleotides in length, alternatively at least about 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 nucleotides in length, wherein in this context the term “about” means the referenced nucleotide sequence length plus or minus 10% of that referenced length. It is noted that novel fragments of a tissue-derived glycoprotein-encoding nucleotide sequence may be determined in a routine manner by aligning the tissue-derived glycoprotein-encoding nucleotide sequence with other known nucleotide sequences using any of a number of well known sequence alignment programs and determining which tissue-derived glycoprotein-encoding nucleotide sequence fragment(s) are novel. All of such novel fragments of tissue-derived glycoprotein-encoding nucleotide sequences are contemplated herein. Also contemplated are the tissue-derived glycoprotein fragments encoded by these nucleotide molecule fragments, preferably those tissue-derived glycoprotein fragments that comprise a binding site for an anti-tissue-derived antibody, a tissue-derived binding oligopeptide or other small organic molecule that binds to a tissue-derived glycoprotein or glycosite.

Thus, in addition to detection of glycoproteins that are tissue-derived either in blood, tissue sample or biological fluid, nucleic acid detection techniques offer additional advantages due to sensitivity of detection. RNA can be collected and/or generated from blood, biological fluids, tissues, organs, cell lines, or other relevant sample using techniques known in the art, such as those described in Kingston. (2002 Current Protocols in Molecular Biology, Greene Publ. Assoc. Inc. & John Wiley & Sons, Inc., NY, N.Y. (see, e.g., as described by Nelson et al. Proc Natl Acad Sci USA, 99:11890-11895, 2002) and elsewhere. Further, a variety of commercially available kits for constructing RNA are useful for making the RNA to be used in the present invention. RNA is constructed from organs/tissues/cells procured from normal healthy subjects; however, this invention contemplates construction of RNA from diseased subjects. This invention contemplates using any type of tissue from any type of subject or animal. For test samples RNA may be procured from an individual (e.g., any animal, including mammals) with or without visible disease and from tissue samples, biological fluids (e.g., whole blood) or the like. In some embodiments amplification or construction of cDNA sequences may be helpful to increase detection capabilities. The present invention, as well as the art, provides the requisite level of detail to perform such tasks. In one aspect of the present invention, whole blood is used as the source of RNA and accordingly, RNA stabilizing regeants are optionally used, such as PAX tubes, as described in Thach et al., J. Immunol. Methods. December 283(1-2):269-279, 2003 and Chai et al., J. Clin. Lab Anal. 19(5):182-188, 2005 (both of which are incorporated herein by reference in their entirety).

Complementary DNA (cDNA) libraries can be generated using techniques known in the art, such as those described in Ausubel et al. (2001 Current Protocols in Molecular Biology, Greene Publ. Assoc. Inc. & John Wiley & Sons, Inc., NY, N.Y.); Sambrook et al. (1989 Molecular Cloning, Second Ed., Cold Spring Harbor Laboratory, Plainview, N.Y.); Maniatis et al. (1982 Molecular Cloning, Cold Spring Harbor Laboratory, Plainview, N.Y.) and elsewhere. Further, a variety of commercially available kits for constructing cDNA libraries are useful for making the cDNA libraries of the present invention. Libraries are constructed from organs/tissues/cells procured from normal, healthy subjects.

Amplification or Nucleic Acid Amplification

By “amplification” or “nucleic acid amplification” is meant production of multiple copies of a target nucleic acid that contains at least a portion of the intended specific target nucleic acid sequence. The multiple copies may be referred to as amplicons or amplification products. In certain embodiments, the amplified target contains less than the complete target gene sequence (introns and exons) or an expressed target gene sequence (spliced transcript of exons and flanking untranslated sequences). For example, specific amplicons may be produced by amplifying a portion of the target polynucleotide by using amplification primers that hybridize to, and initiate polymerization from, internal positions of the target polynucleotide. Preferably, the amplified portion contains a detectable target sequence that may be detected using any of a variety of well-known methods.

Many well-known methods of nucleic acid amplification require thermocycling to alternately denature double-stranded nucleic acids and hybridize primers; however, other well-known methods of nucleic acid amplification are isothermal. The polymerase chain reaction (U.S. Pat. Nos. 4,683,195; 4,683,202; 4,800,159; 4,965,188), commonly referred to as PCR, uses multiple cycles of denaturation, annealing of primer pairs to opposite strands, and primer extension to exponentially increase copy numbers of the target sequence. In a variation called RT-PCR, reverse transcriptase (RT) is used to make a complementary DNA (cDNA) from mRNA, and the cDNA is then amplified by PCR to produce multiple copies of DNA. The ligase chain reaction (Weiss, R. 1991, Science 254: 1292), commonly referred to as LCR, uses two sets of complementary DNA oligonucleotides that hybridize to adjacent regions of the target nucleic acid. The DNA oligonucleotides are covalently linked by a DNA ligase in repeated cycles of thermal denaturation, hybridization and ligation to produce a detectable double-stranded ligated oligonucleotide product. Another method is strand displacement amplification (Walker, G. et al., 1992, Proc. Natl. Acad. Sci. USA 89:392-396; U.S. Pat. Nos. 5,270,184 and 5,455,166), commonly referred to as SDA, which uses cycles of annealing pairs of primer sequences to opposite strands of a target sequence, primer extension in the presence of a dNTPαS to produce a duplex hemiphosphorothioated primer extension product, endonuclease-mediated nicking of a hemimodified restriction endonuclease recognition site, and polymerase-mediated primer extension from the 3′ end of the nick to displace an existing strand and produce a strand for the next round of primer annealing, nicking and strand displacement, resulting in geometric amplification of product. Thermophilic SDA (tSDA) uses thermophilic endonucleases and polymerases at higher temperatures in essentially the same method (European Pat. No. 0 684 315). Other amplification methods include: nucleic acid sequence based amplification (U.S. Pat. No. 5,130,238), commonly referred to as NASBA; one that uses an RNA replicase to amplify the probe molecule itself (Lizardi, P. et al., 1988, BioTechnol. 6: 1197-1202), commonly referred to as Qβ replicase; a transcription based amplification method (Kwoh, D. et al., 1989, Proc. Natl. Acad. Sci. USA 86:1173-1177); self-sustained sequence replication (Guatelli, J. et al., 1990, Proc. Natl. Acad. Sci. USA 87: 1874-1878); and, transcription mediated amplification (U.S. Pat. Nos. 5,480,784 and 5,399,491), commonly referred to as TMA. For further discussion of known amplification methods see Persing, David H., 1993, “In Vitro Nucleic Acid Amplification Techniques” in Diagnostic Medical Microbiology: Principles and Applications (Persing et al., Eds.), pp. 51-87 (American Society for Microbiology, Washington, D.C.).

Other suitable amplification methods include transcription amplification (Kwoh et al., Proc. Natl. Acad. Sci. USA 86, 1173 (1989) and WO88/10315), self-sustained sequence replication (Guatelli et al., Proc. Nat. Acad. Sci. USA, 87, 1874 (1990) and WO90/06995), selective amplification of target polynucleotide sequences (U.S. Pat. No. 6,410,276), consensus sequence primed polymerase chain reaction (CP-PCR) (U.S. Pat. No. 4,437,975), arbitrarily primed polymerase chain reaction (AP-PCR) (U.S. Pat. Nos. 5,413,909, 5,861,245) nucleic acid based sequence amplification (NABSA), rolling circle amplification (RCA), multiple displacement amplification (MDA) (U.S. Pat. Nos. 6,124,120 and 6,323,009) and circle-to-circle amplification (C2CA) (Dahl et al. Proc. Natl. Acad. Sci 101:4548-4553 (2004). (See, U.S. Pat. Nos. 5,409,818, 5,554,517, and 6,063,603, each of which is incorporated herein by reference). Other amplification methods that may be used are described in, U.S. Pat. Nos. 5,242,794, 5,494,810, 5,409,818, 4,988,617, 6,063,603 and 5,554,517 and in U.S. Ser. No. 09/854,317, each of which is incorporated herein by reference.

Additional methods of sample preparation and techniques for reducing the complexity of a nucleic sample are described in Dong et al., Genome Research 11, 1418 (2001), in U.S. Pat. Nos. 6,361,947, 6,391,592 and U.S. Ser. Nos. 09/916,135, 09/920,491 (U.S. Patent Application Publication 20030096235), Ser. No. 09/910,292 (U.S. Patent Application Publication 20030082543), and Ser. No. 10/013,598.

In more particular embodiments, the amplification technique used in the methods of the present invention is a transcription-based amplification technique, such as TMA and NASBA.

Illustrative transcription-based amplification systems of the present invention include TMA, which employs an RNA polymerase to produce multiple RNA transcripts of a target region (U.S. Pat. Nos. 5,480,784 and 5,399,491). TMA uses a “promoter-primer” that hybridizes to a target nucleic acid in the presence of a reverse transcriptase and an RNA polymerase to form a double-stranded promoter from which the RNA polymerase produces RNA transcripts. These transcripts can become templates for further rounds of TMA in the presence of a second primer capable of hybridizing to the RNA transcripts. Unlike PCR, LCR or other methods that require heat denaturation, TMA is an isothermal method that uses an RNase H activity to digest the RNA strand of an RNA:DNA hybrid, thereby making the DNA strand available for hybridization with a primer or promoter-primer. Generally, the RNase H activity associated with the reverse transcriptase provided for amplification is used.

In an illustrative TMA method, one amplification primer is an oligonucleotide promoter-primer that comprises a promoter sequence which becomes functional when double-stranded, located 5′ of a target-binding sequence, which is capable of hybridizing to a binding site of a target RNA at a location 3′ to the sequence to be amplified. A promoter-primer may be referred to as a “T7-primer” when it is specific for T7 RNA polymerase recognition. Under certain circumstances, the 3′ end of a promoter-primer, or a subpopulation of such promoter-primers, may be modified to block or reduce primer extension. From an unmodified promoter-primer, reverse transcriptase creates a cDNA copy of the target RNA, while RNase H activity degrades the target RNA. A second amplification primer then binds to the cDNA. This primer may be referred to as a “non-T7 primer” to distinguish it from a “T7-primer”. From this second amplification primer, reverse transcriptase creates another DNA strand, resulting in a double-stranded DNA with a functional promoter at one end. When double-stranded, the promoter sequence is capable of binding an RNA polymerase to begin transcription of the target sequence to which the promoter-primer is hybridized. An RNA polymerase uses this promoter sequence to produce multiple RNA transcripts (i.e., amplicons), generally about 100 to 1,000 copies. Each newly-synthesized amplicon can anneal with the second amplification primer. Reverse transcriptase can then create a DNA copy, while the RNase H activity degrades the RNA of this RNA:DNA duplex. The promoter-primer can then bind to the newly synthesized DNA, allowing the reverse transcriptase to create a double-stranded DNA, from which the RNA polymerase produces multiple amplicons. Thus, a billion-fold isothermic amplification can be achieved using two amplification primers.

“Selective amplification”, as used herein, refers to the amplification of a target nucleic acid sequence according to the present invention wherein detectable amplification of the target sequence is substantially limited to amplification of target sequence contributed by a nucleic acid sample of interest that is being tested and is not contributed by target nucleic acid sequence contributed by some other sample source, e.g., contamination present in reagents used during amplification reactions or in the environment in which amplification reactions are performed.

By “amplification conditions” is meant conditions permitting nucleic acid amplification according to the present invention. Amplification conditions may, in some embodiments, be less stringent than “stringent hybridization conditions” as described herein. Oligonucleotides used in the amplification reactions of the present invention hybridize to their intended targets under amplification conditions, but may or may not hybridize under stringent hybridization conditions. On the other hand, detection probes of the present invention hybridize under stringent hybridization conditions. While the Examples section infra provides preferred amplification conditions for amplifying target nucleic acid sequences according to the present invention, other acceptable conditions to carry out nucleic acid amplifications according to the present invention could be easily ascertained by someone having ordinary skill in the art depending on the particular method of amplification employed.

Oligonucleotides & Primers for Amplification

As used herein, the term “oligonucleotide” or “oligo” or “oligomer” is intended to encompass a singular “oligonucleotide” as well as plural “oligonucleotides,” and refers to any polymer of two or more of nucleotides, nucleosides, nucleobases or related compounds used as a reagent in the amplification methods of the present invention, as well as subsequent detection methods. The oligonucleotide may be DNA and/or RNA and/or analogs thereof. The term oligonucleotide does not denote any particular function to the reagent, rather, it is used generically to cover all such reagents described herein. An oligonucleotide may serve various different functions, e.g., it may function as a primer if it is capable of hybridizing to a complementary strand and can further be extended in the presence of a nucleic acid polymerase, it may provide a promoter if it contains a sequence recognized by an RNA polymerase and allows for transcription, and it may function to prevent hybridization or impede primer extension if appropriately situated and/or modified. Specific oligonucleotides of the present invention are described in more detail below, but are directed to binding the tissue-derived transcript or the tissue-derived transcript encoding the sequences listed in the attached Table 1 or the appended sequence listing. As used herein, an oligonucleotide can be virtually any length, limited only by its specific function in the amplification reaction or in detecting an amplification product of the amplification reaction.

Oligonucleotides of a defined sequence and chemical structure may be produced by techniques known to those of ordinary skill in the art, such as by chemical or biochemical synthesis, and by in vitro or in vivo expression from recombinant nucleic acid molecules, e.g., bacterial or viral vectors. As intended by this disclosure, an oligonucleotide does not consist solely of wild-type chromosomal DNA or the in vivo transcription products thereof.

Oligonucleotides may be modified in any way, as long as a given modification is compatible with the desired function of a given oligonucleotide. One of ordinary skill in the art can easily determine whether a given modification is suitable or desired for any given oligonucleotide of the present invention. Modifications include base modifications, sugar modifications or backbone modifications. Base modifications include, but are not limited to the use of the following bases in addition to adenine, cytidine, guanosine, thymine and uracil: C-5 propyne, 2-amino adenine, 5-methyl cytidine, inosine, and dP and dK bases. The sugar groups of the nucleoside subunits may be ribose, deoxyribose and analogs thereof, including, for example, ribonucleosides having a 2′-O-methyl substitution to the ribofuranosyl moiety. See Becker et al., U.S. Pat. No. 6,130,038. Other sugar modifications include, but are not limited to 2′-amino, 2′-fluoro, (L)-alpha-threofuranosyl, and pentopuranosyl modifications. The nucleoside subunits may by joined by linkages such as phosphodiester linkages, modified linkages or by non-nucleotide moieties which do not prevent hybridization of the oligonucleotide to its complementary target nucleic acid sequence. Modified linkages include those linkages in which a standard phosphodiester linkage is replaced with a different linkage, such as a phosphorothioate linkage or a methylphosphonate linkage. The nucleobase subunits may be joined, for example, by replacing the natural deoxyribose phosphate backbone of DNA with a pseudo peptide backbone, such as a 2-aminoethylglycine backbone which couples the nucleobase subunits by means of a carboxymethyl linker to the central secondary amine. (DNA analogs having a pseudo peptide backbone are commonly referred to as “peptide nucleic acids” or “PNA” and are disclosed by Nielsen et al., “Peptide Nucleic Acids,” U.S. Pat. No. 5,539,082.) Other linkage modifications include, but are not limited to, morpholino bonds.

Non-limiting examples of oligonucleotides or oligomers contemplated by the present invention include nucleic acid analogs containing bicyclic and tricyclic nucleoside and nucleotide analogs (LNAs). See Imanishi et al., U.S. Pat. No. 6,268,490; and Wengel et al., U.S. Pat. No. 6,670,461.) Any nucleic acid analog is contemplated by the present invention provided the modified oligonucleotide can perform its intended function, e.g., hybridize to a target nucleic acid under stringent hybridization conditions or amplification conditions, or interact with a DNA or RNA polymerase, thereby initiating extension or transcription. In the case of detection probes, the modified oligonucleotides must also be capable of preferentially hybridizing to the target nucleic acid under stringent hybridization conditions.

While design and sequence of oligonucleotides for the present invention depend on their function as described below, several variables must generally be taken into account. Among the most critical are: length, melting temperature (Tm), specificity, complementarity with other oligonucleotides in the system, G/C content, polypyrimidine (T, C) or polypurine (A, G) stretches, and the 3′-end sequence. Controlling for these and other variables is a standard and well known aspect of oligonucleotide design, and various computer programs are readily available to screen large numbers of potential oligonucleotides for optimal ones.

The 3′-terminus of an oligonucleotide (or other nucleic acid) can be blocked in a variety of ways using a blocking moiety, as described below. A “blocked” oligonucleotide is not efficiently extended by the addition of nucleotides to its 3′-terminus, by a DNA- or RNA-dependent DNA polymerase, to produce a complementary strand of DNA. As such, a “blocked” oligonucleotide cannot be a “primer.”

As used in this disclosure, the phrase “an oligonucleotide having a nucleic acid sequence ‘comprising,’ ‘consisting of,’ or ‘consisting essentially of’ a sequence selected from” a group of specific sequences means that the oligonucleotide, as a basic and novel characteristic, is capable of stably hybridizing to a nucleic acid having the exact complement of one of the listed nucleic acid sequences of the group under stringent hybridization conditions. An exact complement includes the corresponding DNA or RNA sequence.

The phrase “an oligonucleotide substantially corresponding to a nucleic acid sequence” means that the referred to oligonucleotide is sufficiently similar to the reference nucleic acid sequence such that the oligonucleotide has similar hybridization properties to the reference nucleic acid sequence in that it would hybridize with the same target nucleic acid sequence under stringent hybridization conditions.

One skilled in the art will understand that “substantially corresponding” oligonucleotides of the invention can vary from the referred to sequence and still hybridize to the same target nucleic acid sequence. This variation from the nucleic acid may be stated in terms of a percentage of identical bases within the sequence or the percentage of perfectly complementary bases between the probe or primer and its target sequence. Thus, an oligonucleotide of the present invention substantially corresponds to a reference nucleic acid sequence if these percentages of base identity or complementarity are from 100% to about 80%. In certain embodiments, the percentage is from 100% to about 85%. In other embodiments, this percentage can be from 100% to about 90%; in further embodiments, this percentage is from 100% to about 95%. One skilled in the art will understand the various modifications to the hybridization/annealing conditions that might be required at various percentages of complementarity to allow hybridization to a specific target sequence without causing an unacceptable level of non-specific hybridization.

The term “mRNA” or sometimes refer by “mRNA transcripts” as used herein, include, but not limited to pre-mRNA transcript(s), transcript processing intermediates, mature mRNA(s) ready for translation and transcripts of the gene or genes, or nucleic acids derived from the mRNA transcript(s). Transcript processing may include splicing, editing and degradation. As used herein, a nucleic acid derived from an mRNA transcript refers to a nucleic acid for whose synthesis the mRNA transcript or a subsequence thereof has ultimately served as a template. Thus, a cDNA reverse transcribed from an mRNA, an RNA transcribed from that cDNA, a DNA amplified from the cDNA, an RNA transcribed from the amplified DNA, etc., are all derived from the mRNA transcript and detection of such derived products is indicative of the presence and/or abundance of the original transcript in a sample. Thus, mRNA derived samples include, but are not limited to, mRNA transcripts of the gene or genes, cDNA reverse transcribed from the mRNA, cRNA transcribed from the cDNA, DNA amplified from the genes, RNA transcribed from amplified DNA, and the like.

The term “nucleic acid library” or sometimes refer by “array” as used herein refers to an intentionally created collection of nucleic acids which can be prepared either synthetically or biosynthetically and screened for biological activity in a variety of different formats (for example, libraries of soluble molecules; and libraries of oligos tethered to resin beads, silica chips, or other solid supports). Additionally, the term “array” is meant to include those libraries of nucleic acids which can be prepared by spotting nucleic acids of essentially any length (for example, from 1 to about 1000 nucleotide monomers in length) onto a substrate. The term “nucleic acid” as used herein refers to a polymeric form of nucleotides of any length, either ribonucleotides, deoxyribonucleotides or peptide nucleic acids (PNAs), that comprise purine and pyrimidine bases, or other natural, chemically or biochemically modified, non-natural, or derivatized nucleotide bases. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. Thus the terms nucleoside, nucleotide, deoxynucleoside and deoxynucleotide generally include analogs such as those described herein. These analogs are those molecules having some structural features in common with a naturally occurring nucleoside or nucleotide such that when incorporated into a nucleic acid or oligonucleoside sequence, they allow hybridization with a naturally occurring nucleic acid sequence in solution. Typically, these analogs are derived from naturally occurring nucleosides and nucleotides by replacing and/or modifying the base, the ribose or the phosphodiester moiety. The changes can be tailor made to stabilize or destabilize hybrid formation or enhance the specificity of hybridization with a complementary nucleic acid sequence as desired.

The term “nucleic acids” as used herein may include any polymer or oligomer of pyrimidine and purine bases, preferably cytosine, thymine, and uracil, and adenine and guanine, respectively. See Albert L. Lehninger, PRINCIPLES OF BIOCHEMISTRY, at 793-800 (Worth Pub. 1982). Indeed, the present invention contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally-occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

When referring to arrays and microarrays the term “oligonucleotide” or sometimes refer by “polynucleotide” as used herein refers to a nucleic acid ranging from at least 2, preferable at least 8, and more preferably at least 20 nucleotides in length or a compound that specifically hybridizes to a polynucleotide. Polynucleotides of the present invention include sequences of deoxyribonucleic acid (DNA) or ribonucleic acid (RNA) which may be isolated from natural sources, recombinantly produced or artificially synthesized and mimetics thereof. A further example of a polynucleotide of the present invention may be peptide nucleic acid (PNA). The invention also encompasses situations in which there is a nontraditional base pairing such as Hoogsteen base pairing which has been identified in certain tRNA molecules and postulated to exist in a triple helix. “Polynucleotide” and “oligonucleotide” are used interchangeably in this application.

The term “primer” as used herein refers to a single-stranded oligonucleotide capable of acting as a point of initiation for template-directed DNA synthesis under suitable conditions for example, buffer and temperature, in the presence of four different nucleoside triphosphates and an agent for polymerization, such as, for example, DNA or RNA polymerase or reverse transcriptase. The length of the primer, in any given case, depends on, for example, the intended use of the primer, and generally ranges from 15 to 30 nucleotides. Short primer molecules generally require cooler temperatures to form sufficiently stable hybrid complexes with the template. A primer need not reflect the exact sequence of the template but must be sufficiently complementary to hybridize with such template. The primer site is the area of the template to which a primer hybridizes. The primer pair is a set of primers including a 5′ upstream primer that hybridizes with the 5′ end of the sequence to be amplified and a 3′ downstream primer that hybridizes with the complement of the 3′ end of the sequence to be amplified.

The term “probe” as used herein refers to a surface-immobilized molecule that can be recognized by a particular target. See U.S. Pat. No. 6,582,908 for an example of arrays having all possible combinations of probes with 10, 12, and more bases. Examples of probes that can be investigated by this invention include, but are not restricted to, agonists and antagonists for cell membrane receptors, toxins and venoms, viral epitopes, hormones (for example, opioid peptides, steroids, etc.), hormone receptors, peptides, enzymes, enzyme substrates, cofactors, drugs, lectins, sugars, oligonucleotides, nucleic acids, oligosaccharides, proteins, and monoclonal antibodies.

The present invention provides a diverse population of uniquely labeled probes in which a target specific nucleic acid contains a nucleic acid bound to a unique label. In addition, the invention provides a diverse population of uniquely labeled probes containing two attached populations of nucleic acids, one population of nucleic acids containing thirty or more target specific nucleic acid probes, and a second population of nucleic acids containing a nucleic acid bound by a unique label.

A target specific probe is intended to mean an agent that binds to the target analyte selectively. This agent will bind with preferential affinity toward the target while showing little to no detectable cross-reactivity toward other molecules.

The target analyte can be any type of macromolecule, including a nucleic acid, a protein or even a small molecule drug. For example, a target can be a nucleic acid that is recognized and bound specifically by a complementary nucleic acid including for example, an oligonucleotide or a PCR product, or a non-natural nucleic acid such as a locked nucleic acid (LNA) or a peptide nucleic acid (PNA). In addition, a target can be a peptide that is bound by a nucleic acid. For example, a DNA binding domain of a transcription factor can bind specifically to a particular nucleic acid sequence. Another example of a peptide that can be bound by a nucleic acid is a peptide that can be bound by an aptamer. Aptamers are nucleic acid sequences that have three dimensional structures capable of binding small molecular targets including metal ions, organic dyes, drugs, amino acids, co-factors, aminoglycosides, antibiotics, nucleotide base analogs, nucleotides and peptides (Jayasena, S. D., Clinical Chemistry 45:9, 1628-1650, (1999)) incorporated herein by reference. Further, a target can be a peptide that is bound by another peptide or an antibody or antibody fragment. The binding peptide or antibody can be linked to a nucleic acid, for example, by the use of known chemistries including chemical and UV cross-linking agents. In addition, a peptide can be linked to a nucleic acid through the use of an aptamer that specifically binds the peptide. Other nucleic acids can be directly attached to the aptamer or attached through the use of hybridization. A target molecule can even be a small molecule that can be bound by an aptamer or a peptide ligand binding domain.

The invention further provides a method for detecting a nucleic acid analyte, by contacting a mixture of nucleic acid analytes with a population of target specific probes each attached to a unique label under conditions sufficient for hybridization of the probes to the target and measuring the resulting signal from one or more of the target specific probes hybridized to an analyte where the signal uniquely identifies the analyte.

The nucleic acid analyte can contain any type of nucleic acid, including for example, an RNA population or a population of cDNA copies. The invention provides for at least one target specific probe for each analyte in a mixture. The invention also provides for a target specific probe that contains a nucleic acid bound to a unique label. Furthermore, the invention provides two attached populations of nucleic acids, one population of nucleic acids containing a plurality of target specific nucleic acid probes, and a second population of nucleic acids containing a nucleic acid bound by a unique label. When the target specific probes are attached to unique labels, this allows for the unique identification of the target analytes.

Methods for conducting polynucleotide hybridization assays have been well developed in the art. Hybridization assay procedures and conditions will vary depending on the application and are selected in accordance with the general binding methods known including those referred to in: Maniatis et al. Molecular Cloning: A Laboratory Manual (2nd Ed. Cold Spring Harbor, N.Y., 1989); Berger and Kimmel Methods in Enzymology, Vol. 152, Guide to Molecular Cloning Techniques (Academic Press, Inc., San Diego, Calif., 1987); Young and Davism, P.N.A.S, 80: 1194 (1983). Methods and apparatus for carrying out repeated and controlled hybridization reactions have been described in U.S. Pat. Nos. 5,871,928, 5,874,219, 6,045,996 and 6,386,749, 6,391,623 each of which are incorporated herein by reference

The present invention also contemplates signal detection of hybridization between ligands in certain preferred embodiments. See U.S. Pat. Nos. 5,143,854, 5,578,832; 5,631,734; 5,834,758; 5,936,324; 5,981,956; 6,025,601; 6,141,096; 6,185,030; 6,201,639; 6,218,803; and 6,225,625, in U.S. Ser. No. 10/389,194 and in PCT Application PCT/US99/06097 (published as WO99/47964), each of which also is hereby incorporated by reference in its entirety for all purposes.

Methods and apparatus for signal detection and processing of intensity data are disclosed in, for example, U.S. Pat. Nos. 5,143,854, 5,547,839, 5,578,832, 5,631,734, 5,800,992, 5,834,758; 5,856,092, 5,902,723, 5,936,324, 5,981,956, 6,025,601, 6,090,555, 6,141,096, 6,185,030, 6,201,639; 6,218,803; and 6,225,625, in U.S. Ser. Nos. 10/389,194, 60/493,495 and in PCT Application PCT/US99/06097 (published as WO99/47964), each of which also is hereby incorporated by reference in its entirety for all purposes.

The practice of the present invention may also employ conventional biology methods, software and systems. Computer software products of the invention typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and etc. The computer executable instructions may be written in a suitable computer language or combination of several languages. Basic computational biology methods are described in, for example Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2nd ed., 2001). See U.S. Pat. No. 6,420,108.

The present invention may also make use of various computer program products and software for a variety of purposes, such as probe design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170.

The whole genome sampling assay (WGSA) is described, for example in Kennedy et al., Nat. Biotech. 21, 1233-1237 (2003), Matsuzaki et al., Gen. Res. 14:414-425, (2004), and Matsuzaki, et al. Nature Methods 1:109-111 (2004). Algorithms for use with mapping assays are described, for example, in Liu et al., Bioinformatics 19: 2397-2403 (2003) and Di et al. Bioinformatics 21:1958 (2005). Additional methods related to WGSA and arrays useful for WGSA and applications of WGSA are disclosed, for example, in U.S. Patent Application Nos. 60/676,058 filed Apr. 29, 2005, 60/616,273 filed Oct. 5, 2004, Ser. Nos. 10/912,445, 11/044,831, 10/442,021, 10/650,332 and 10/463,991. Genome wide association studies using mapping assays are described in, for example, Hu et al., Cancer Res.; 65(7):2542-6 (2005), Mitra et al., Cancer Res., 64(21):8116-25 (2004), Butcher et al., Hum Mol Genet., 14(10):1315-25 (2005), and Klein et al., Science, 308(5720):385-9 (2005). Each of these references is incorporated herein by reference in its entirety for all purposes.

Additionally, the present invention may have preferred embodiments that include methods for providing genetic information over networks such as the Internet as shown in U.S. Ser. Nos. 10/197,621, 10/063,559 (United States Publication Number 20020183936), Ser. Nos. 10/065,856, 10/065,868, 10/328,818, 10/328,872, 10/423,403, and 60/482,389.

The term “array” as used herein refers to an intentionally created collection of molecules that can be prepared either synthetically or biosynthetically. The molecules in the array can be identical or different from each other. The array can assume a variety of formats, for example, libraries of soluble molecules; libraries of compounds tethered to resin beads, silica chips, or other solid supports.

Methods of Use

The present invention provides tissue-derived glycoprotein, glycosite and transcript sets and normal serum tissue-derived glycoprotein, glycosite and transcript sets, panels thereof, detection reagents and probes directed thereto and methods for using and identifying the same. The present invention further provides panels, arrays, mixtures, and kits comprising detection reagents or probes for detecting such glycoproteins, glycosites, or polynucleotides that encode them in blood, other bodily fluid, and tissue samples such as biopsy samples from diseased organs.

It should also be understood that the blood glycoprotein and transcript fingerprints constitute assays for the normal tissue and all the diseases of the tissue. Thus all different diseases affecting such tissues either directly or indirectly may be detected or monitored because each different type of disease arises from distinct disease-perturbed networks that change the levels of different combinations of glycoproteins whose synthesis they control. The present invention is not claiming disease-specific glycoproteins, rather the fingerprints report the tissue status for all different normal and disease tissue conditions. Thus, the diagnostic panels and generally, methods used for detecting normal serum tissue-derived glycoproteins, can be used to define/identify disease-associated tissue-derived serum glycoprotein fingerprints.

The present invention provides methods for identifying tissue- and plasma-derived glycosites and the glycoproteins containing those glycosites and methods for identifying tissue-derived serum glycoprotein fingerprints. The present invention further provides panels/arrays of detection reagents for detecting tissue-derived glycoproteins and glycosites and tissue-derived serum glycoprotein or glycosite sets thereof. The present invention also provides defined tissue-derived glycoprotein blood fingerprints for normal and disease settings. As such, the present invention provides methods of detecting and diagnosing diseases. The invention further provides methods for stratifying disease types and for monitoring the progression of a disease. The present invention also provides for following responses to therapy in a variety of disease settings and methods for detecting the disease state in humans using the visualization of nanoparticles with appropriate reporter groups, antibodies or aptamers.

The present invention can be used as a standard screening test. In this regard, one or more of the diagnostic/prognostic panels described herein can be run on an individual and any statistically significant deviation from a normal tissue-derived glycoprotein blood fingerprint would indicate that disease-related perturbation was present. Thus, the present invention provides a standard or “normal” blood fingerprint for any given tissue. In certain embodiments, a normal blood fingerprint is determined by measuring the normal range of levels of the individual protein members of a fingerprint. Any deviation therefrom or perturbation of the normal fingerprint that is outside the standard deviation (normal range) has diagnostic utility (see also U.S. Patent Application No. 0020095259). As would be recognized by the skilled artisan, the significance of any deviation in the levels of (e.g., a significantly altered level of one or more of) the individual protein members of a fingerprint can be determined using statistical methods known in the art and described herein. As noted elsewhere herein, perturbation of the normal fingerprint can indicate primary disease of the tissue being tested or secondary, indirect affects on that tissue resulting from disease of another tissue.

In an additional embodiment, the present invention can be used to determine distinct normal tissue-derived glycoprotein blood fingerprints, such as in different populations of people. In this regard, distinct normal patterns of tissue-derived glycoprotein blood fingerprints may have differences in populations of patients that permit one to stratify patients into classes that would respond to a particular therapeutic regimen and those which would not.

In a further embodiment, the present invention can be used to determine the risk of developing a particular biological condition. A statistically significant alteration (e.g., increase or decrease) in the levels of one or more members of a particular tissue-derived glycoprotein blood fingerprint may signify a risk of developing a particular disease, such as a cancer, an autoimmune disease, or other biological condition.

To monitor the progression of a disease, or monitor responses to therapy, one or more tissue-derived glycoprotein blood fingerprints are detected/measured as described herein using any of the methods as described herein at one time point and detected/measured again at subsequent time points, thereby monitoring disease progression or responses to therapy.

The present invention further provides methods of identifying new drug targets for a disease or indication by detecting specific up-regulation of a transcript or polypeptide in a diseased state. In addition, the present invention contemplates using such targets for imaging or drug targeting such that a probe to a disease specific glycoprotein or transcript may be utilized alone as a targeting agent or coupled to another therapeutic or diagnostic imaging agent.

The normal tissue-derived glycoprotein blood fingerprints of the present invention can be used as a baseline for detecting any of a variety of diseases (or the lack thereof). In certain embodiments, the tissue-derived glycoprotein blood fingerprints of the present invention can be used to detect cancer. As such, the present invention can be used to detect, monitor progression of, or monitor therapeutic regimens for any cancer, including melanoma, non-Hodgkin's lymphoma, Hodgkin's disease, leukemias, plasmocytomas, sarcomas, adenomas, gliomas, thymomas, breast cancer, prostate cancer, colo-rectal cancer, kidney cancer, renal cell carcinoma, uterine cancer, pancreatic cancer, esophageal cancer, brain cancer, lung cancer, ovarian cancer, cervical cancer, testicular cancer, gastric cancer, multiple myeloma, hepatoma, acute lymphoblastic leukemia (ALL), acute myelogenous leukemia (AML), chronic myelogenous leukemia (CML), and chronic lymphocytic leukemia (CLL), or other cancers.

In certain embodiments, the tissue-derived glycoprotein blood fingerprints of the present invention can be used to detect, to monitor progression of, or monitor therapeutic regimens for diseases of the heart, kidney, ureter, bladder, urethra, liver, prostate, heart, blood vessels, bone marrow, skeletal muscle, smooth muscle, various specific regions of the brain (including, but not limited to the amygdala, caudatenucleus, cerebellum, corpuscallosum, fetal, hypothalamus, thalamus), spinal cord, peripheral nerves, retina, nose, trachea, lungs, mouth, salivary gland, esophagus, stomach, small intestines, large intestines, hypothalamus, pituitary, thyroid, pancreas, adrenal glands, ovaries, oviducts, uterus, placenta, vagina, mammary glands, testes, seminal vesicles, penis, lymph nodes, thymus, and spleen. The present invention can be used to detect, to monitor progression of, or monitor therapeutic regimens for cardiovascular diseases, neurological diseases, metabolic diseases, respiratory diseases, autoimmune diseases. As would be recognized by the skilled artisan, the present invention can be used to detect, monitor the progression of, or monitor treatment for, virtually any disease wherein the disease causes perturbation in tissue-derived serum glycoproteins.

In certain embodiments, the tissue-derived glycoprotein blood fingerprints of the present invention can be used to detect autoimmune disease. As such, the present invention can be used to detect, monitor progression of, or monitor therapeutic regimens for autoimmune diseases such as, but not limited to, rheumatoid arthritis, multiple sclerosis, insulin dependent diabetes, Addison's disease, celiac disease, chronic fatigue syndrome, inflammatory bowel disease, ulcerative colitis, Crohn's disease, Fibromyalgia, systemic lupus erythematosus, psoriasis, Sjogren's syndrome, hyperthyroidism/Graves disease, hypothyroidism/Hashimoto's disease, Insulin-dependent diabetes (type 1), Myasthenia Gravis, endometriosis, scleroderma, pernicious anemia, Goodpasture syndrome, Wegener's disease, glomerulonephritis, aplastic anemia, paroxysmal nocturnal hemoglobinuria, myelodysplastic syndrome, idiopathic thrombocytopenic purpura, autoimmune hemolytic anemia, Evan's syndrome, Factor VIII inhibitor syndrome, systemic vasculitis, dermatomyositis, polymyositis and rheumatic fever.

In certain embodiments, the tissue-derived glycoprotein blood fingerprints of the present invention can be used to detect diseases associated with infections with any of a variety of infectious organisms, such as viruses, bacteria, parasites and fungi. Infectious organisms may comprise viruses, (e.g., RNA viruses, DNA viruses, human immunodeficiency virus (HIV), hepatitis A, B, and C virus, herpes simplex virus (HSV), cytomegalovirus (CMV) Epstein-Barr virus (EBV), human papilloma virus (HPV)), parasites (e.g., protozoan and metazoan pathogens such as Plasmodia species, Leishmania species, Schistosoma species, Trypanosoma species), bacteria (e.g., Mycobacteria, in particular, M. tuberculosis, Salmonella, Streptococci, E. coli, Staphylococci), fungi (e.g., Candida species, Aspergillus species), Pneumocystis carinii, and prions.

One of ordinary skill in the art could readily conclude that the present invention is useful in defining the normal parameters for any number of tissues in the body. To that end, the present invention may also be used to define subclinical perturbations from normal during annual screenings that could be utilized to initiate therapy or more aggressive examinations at an earlier date. Further, defining normal for two, three, or more related tissues can be accomplished by the present invention. Such groupings would be clear to those of skill in the art and could be any of a variety, include those related to cardiovascular health, including the heart, lungs, liver, etc. as well as looking at groupings of liver and blood for infectious and parasitic diseases such as malaria, HIV, and the like.

Using the diagnostic panels and methods described herein, a vast array of disease-associated blood fingerprints can be defined for any of a variety of diseases as described further herein. As such, the present invention further provides information databases comprising data that make up blood fingerprints as described herein. As such, the databases may comprise the defined differential expression levels as determined using any of a variety of methods such as those described herein, of each of the plurality of tissue-derived glycoproteins or glycosites that make up a given fingerprint in any of a variety of settings (e.g., normal or disease fingerprints).

In a still further embodiment, the invention concerns a composition of matter comprising a glycoprotein or glycosite as described herein and listed in the Tables herein, a chimeric glycoprotein or glycosite as described herein, an anti-tissue-derived and/or serum-derived glycoprotein or glycosite antibody as described herein, an oligopeptide as described herein, or an organic molecule as described herein, in combination with a carrier. Optionally, the carrier is a pharmaceutically acceptable carrier.

In yet another embodiment, the invention concerns an article of manufacture comprising a container and a composition of matter contained within the container, wherein the composition of matter may comprise a glycoprotein or glycosite as described herein such as those listed in Table 1, a chimeric tissue- and/or serum-derived glycoprotein or glycosite as described herein, an anti-tissue- and/or serum-derived glycoprotein or glycosite antibody as described herein, a tissue- and/or serum-derived glycoprotein or glycosite oligopeptide as described herein, or a tissue-and/or serum derived glycoprotein or glycosite binding organic molecule as described herein. The article may further optionally comprise a label affixed to the container, or a package insert included with the container, that refers to the use of the composition of matter for the therapeutic treatment or diagnostic detection of a tumor.

Another embodiment of the present invention is directed to the use of glycoprotein or glycosite as described herein, a chimeric glycoprotein or glycosite as described herein, an anti-glycoprotein or glycosite antibody as described herein, a glycoprotein or glycosite binding oligopeptide as described herein, or a glycoprotein or glycosite binding organic molecule as described herein, for the preparation of a medicament useful in the treatment of a condition which is responsive to the glycoprotein or glycosite, chimeric glycoprotein or glycosite, anti-glycoprotein or glycosite antibody, glycoprotein or glycosite binding oligopeptide, or glycoprotein or glycosite binding organic molecule.

Another embodiment of the present invention is directed to a method for inhibiting the growth of a cell that expresses a tissue-derived serum glycoprotein, wherein the method comprises contacting the cell with an antibody, an oligopeptide or a small organic molecule that binds to the tissue-derived serum glycoprotein, and wherein the binding of the antibody, oligopeptide or organic molecule to the tissue-derived serum glycoprotein causes inhibition of the growth of the cell expressing the tissue-derived serum glycoprotein. In preferred embodiments, the cell is a cancer cell or disease harboring cell and binding of the antibody, oligopeptide or organic molecule to the tissue-derived serum glycoprotein causes death of the cell expressing the tissue-derived serum glycoprotein. Optionally, the antibody is a monoclonal antibody, antibody fragment, chimeric antibody, humanized antibody, or single-chain antibody. Antibodies, tissue-derived serum glycoprotein binding oligopeptides and tissue-derived serum glycoprotein binding organic molecules employed in the methods of the present invention may optionally be conjugated to a growth inhibitory agent or cytotoxic agent such as a toxin, including, for example, a maytansinoid or calicheamicin, an antibiotic, a radioactive isotope, a nucleolytic enzyme, or the like. The antibodies and binding oligopeptides employed in the methods of the present invention may optionally be produced in CHO cells or bacterial cells.

Yet another embodiment of the present invention is directed to a method of therapeutically treating a mammal having cancerous cells or disease containing cells or tissues comprising cells that express a tissue-derived serum glycoprotein, wherein the method comprises administering to the mammal a therapeutically effective amount of an antibody, an oligopeptide or a small organic molecule that binds to the tissue-derived serum glycoprotein, thereby resulting in the effective therapeutic treatment of the tumor. Optionally, the antibody is a monoclonal antibody, antibody fragment, chimeric antibody, humanized antibody, or single-chain antibody. Antibodies, binding oligopeptides and binding organic molecules employed in the methods of the present invention may optionally be conjugated to a growth inhibitory agent or cytotoxic agent such as a toxin, including, for example, a maytansinoid or calicheamicin, an antibiotic, a radioactive isotope, a nucleolytic enzyme, or the like. The antibodies and oligopeptides employed in the methods of the present invention may optionally be produced in CHO cells or bacterial cells.

Yet another embodiment of the present invention is directed to a method of determining the presence of any 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100, 200, 300, or more of the glycoproteins or glycosites described herein, such as those listed in Table 1, in a sample suspected of containing the glycoproteins or glycosites, wherein the method comprises exposing the sample to an antibody, oligopeptide or small organic molecule that binds to the glycoprotein or glycosite and determining binding of the antibody, oligopeptide or organic molecule to the glycoprotein or glycosite in the sample, wherein the presence of such binding is indicative of the presence of the glycoprotein or glycosite in the sample. Optionally, the sample may contain cells (which may be cancer cells) suspected of expressing the glycoprotein. The antibody, binding oligopeptide or binding organic molecule employed in the method may optionally be detectably labeled, attached to a solid support, or the like. As such, the present invention provides for a method of determining the presence of any of the glycoproteins or glycosites described herein in a sample suspected of containing the glycoproteins or glycosites, wherein the method comprises exposing the sample to a diagnostic/prognostic panel as described herein and determining binding of the detection reagents of the panel to the glycoprotein or glycosite in the sample, wherein the presence of such binding is indicative of the presence of the glycoprotein or glycosite in the sample.

A further embodiment of the present invention is directed to a method of diagnosing the presence of a tumor in a mammal, wherein the method comprises detecting the level of expression of a gene encoding a glycoprotein or glycosite as described herein (see e.g., Table 1) (a) in a test sample of tissue or cells obtained from said mammal, and (b) in a control sample of known normal non-cancerous cells of the same tissue origin or type, wherein a statistically significant higher or lower level of expression of the gene encoding a glycoprotein or glycosite in the test sample, as compared to the control sample, is indicative of the presence of tumor in the mammal from which the test sample was obtained. The method can be carried out using the diagnostic/prognostic panels as described herein.

Another embodiment of the present invention is directed to a method of diagnosing the presence of a tumor in a mammal, wherein the method comprises (a) contacting a test sample comprising tissue cells obtained from the mammal with an antibody, oligopeptide or small organic molecule that binds to a glycoprotein or glycosite as described herein and (b) detecting the formation of a complex between the antibody, oligopeptide or small organic molecule and the glycoprotein or glycosite in the test sample, wherein the formation of a complex is indicative of the presence of a tumor in the mammal. Optionally, the antibody, binding oligopeptide or binding organic molecule employed is detectably labeled, attached to a solid support, or the like, and/or the test sample of tissue cells is obtained from an individual suspected of having a cancerous tumor. As such, in certain embodiments, the diagnostic/prognostic panels as described herein are used in the method of diagnosing the presence of a tumor in a mammal.

Yet another embodiment of the present invention is directed to a method for treating or preventing a cell proliferative disorder associated with altered, in certain embodiments, increased, expression or activity of a glycoprotein as described herein (see e.g., those listed in Table 1), the method comprising administering to a subject in need of such treatment an effective amount of an antagonist of the glycoprotein. Preferably, the cell proliferative disorder is cancer and the antagonist of the glycopolypeptide is an anti-glycopolypeptide antibody, binding oligopeptide, binding organic molecule or antisense oligonucleotide. Effective treatment or prevention of the cell proliferative disorder may be a result of direct killing or growth inhibition of cells that express a tissue-and/or serum derived glycoprotein or by antagonizing the cell growth potentiating activity of a glycoprotein as described herein.

Yet another embodiment of the present invention is directed to a method of binding an antibody, oligopeptide or small organic molecule to a cell that expresses a glycopolypeptide or glycosite as described herein, wherein the method comprises contacting a cell that expresses the glycoprotein with said antibody, oligopeptide or small organic molecule under conditions which are suitable for binding of the antibody, oligopeptide or small organic molecule to said glycopolypeptide and allowing binding therebetween.

In another embodiment of the present invention, there is a method of diagnosing or prognosing a disease in an individual, comprising the steps of: a) determining the level of one or more glycoprotein as described herein such as in Table 1, or gene transcripts encoding said one or more glycoprotein, in blood obtained from said individual suspected of having a disease, and b) comparing the level of each of said one or more transcripts or glycoproteins in said blood according to step a) with the level of each of said one or more transcripts or protein in blood from one or more individuals having a disease, wherein detecting the same levels of each of said one or more transcripts or proteins in the comparison of step b) is indicative of a disease in the individual of step a).

In another embodiment of the present invention, there is a method of determining a stage of disease progression or regression in an individual having a disease, comprising the steps of: a) determining the level of one or more glycoproteins as described herein such as in Table 1, or gene transcripts encoding said one or more glycoproteins, in blood obtained from said individual having a disease, and b) comparing the level of each of said one or more glycoproteins or gene transcripts in said blood according to step a) with the level of each of said glycoproteins or gene transcripts encoding said glycoproteins in blood obtained from one or more individuals who each have been diagnosed as being at the same progressive or regressive stage of a disease, wherein the comparison from step b) allows the determination of the stage of a disease progression or regression in an individual.

In another embodiment of the present invention, there is a method of diagnosing or determining the prognosis of a disease in an individual, comprising the steps of: a) determining the level of one or more glycoproteins as described herein, such as in Table 1, or gene transcripts encoding said one or more glycoproteins, in blood obtained from said individual suspected of having a disease, and b) comparing the level of each of said one or more transcripts or glycoproteins in said blood according to step a) with a predetermined normal level of each of said one or more transcripts or glycoproteins in blood; wherein detecting a statistically significant altered level (either an increase or a decrease) of each of said one or more transcripts or proteins in the comparison of step b) is indicative of a disease in the individual of step a).

When comparing two or more samples for differences, results are reported as statistically significant when there is only a small probability that similar results would have been observed if the tested hypothesis (i.e., the genes are not expressed at different levels) were true. A small probability can be defined as the accepted threshold level at which the results being compared are considered significantly different. The accepted lower threshold is set at, but not limited to, 0.05 (i.e., there is a 5% likelihood that the results would be observed between two or more identical populations) such that any values determined by statistical means at or below this threshold are considered significant.

When comparing two or more samples for similarities, results are reported as statistically significant when there is only a small probability that similar results would have been observed if the tested hypothesis (i.e., the genes are not expressed at different levels) were true. A small probability can be defined as the accepted threshold level at which the results being compared are considered significantly different. The accepted lower threshold is set at, but not limited to, 0.05 (i.e., there is a 5% likelihood that the results would be observed between two or more identical populations) such that any values determined by statistical means above this threshold are not considered significantly different and thus similar.

Identification of glycoproteins, glycosites, or transcripts encoding such glycoproteins or glycosites as described herein that are differentially expressed in blood samples from patients with disease as compared to healthy patients or as compared to patients without said disease is determined by statistical analysis of the gene or protein expression profiles from healthy patients or patients without disease compared to patients with disease using the Wilcox Mann Whitney rank sum test. Other statistical tests can also be used, see for example (Sokal and Rohlf (1987) Introduction to Biostatistics 2nd edition, W H Freeman, New York), which is incorporated herein in their entirety.

In order to facilitate ready access, e.g., for comparison, review, recovery and/or modification, the expression profiles of patients with disease and/or patients without disease or healthy patients can be recorded in a database, whether in a relational database accessible by a computational device or other format, or a manually accessible indexed file of profiles as photographs, analogue or digital imaging, readouts spreadsheets etc. Typically the database is compiled and maintained at a central facility, with access being available locally and/or remotely.

As would be understood by a person skilled in the art, comparison as between the expression profile of a test patient with expression profiles of patients with a disease, expression profiles of patients with a certain stage or degree of progression of said disease, without said disease, or a healthy patient so as to diagnose or determine the prognosis of said test patient can occur via expression profiles generated concurrently or non concurrently. It would be understood that expression profiles can be stored in a database to allow said comparison.

As additional test samples from test patients are obtained, through clinical trials, further investigation, or the like, additional data can be determined in accordance with the methods disclosed herein and can likewise be added to a database to provide better reference data for comparison of healthy and/or non-disease patients and/or certain stage or degree of progression of a disease as compared with the test patient sample. These and other methods, including those described in the art (e.g., U.S. Patent Application Pub No. 20060134637) can be used in the context of the sequences disclosed.

Business Methods

A further embodiment of the present invention comprises business methods for manufacturing one or more of the detection reagents, panels, arrays as described herein as well as providing diagnostic services for analyzing and/or comparing fingerprints or individual proteins (or nucleic acid molecules) from a subject with one, two or more glycoproteins or glycosites as described herein or nucleic acid molecules described herein, identifying disease-associated fingerprints or glycoproteins, glycosites or nucleic acid molecules that vary or become present with disease, identifying fingerprints or proteins or nucleic acid molecule levels perturbed from normal, providing manufacturers of genomics devices the use of the detection reagents, panels, arrays, tissue-derived serum glycoprotein fingerprints or specific glycoproteins or nucleic acid probes for nucleic acid molecules encoding the same described herein to develop diagnostic devices, where the genomics device includes any device that may be used to define differences in a sample between the normal and disturbed state resulting from one or more effects, providing manufacturers of proteomics devices the use of the detection reagents, panels, arrays, tissue-derived serum glycoproteins or glycosites described herein to develop diagnostic devices, where the proteomics device includes any device that may be used to define differences in a sample between the normal and disturbed state resulting a disease, disorder or therapy, providing manufacturers of imaging devices detection reagents, panels, arrays, lateral flow devices, glycoproteins, glycosites or nucleic acid molecules or probes thereto described herein to develop diagnostic devices, where the proteomics devices include any device that may be used to define differences in a blood sample between the normal and disturbed state resulting from disease, drug side-effects, or therapeutic interventions, providing manufacturers of molecular imaging devices the use of the detection reagents, panels, arrays, or blood fingerprints described herein to develop diagnostic devices, where the proteomics device includes any device that may be used to define differences in a blood sample between the normal and disturbed state and marketing to healthcare providers the benefits of using the detection reagents, panels, arrays, and diagnostic services of the present invention to enhance diagnostic capabilities and thus, to better treat patients.

Also provided is an aspect of the invention to utilize databases to store data and analysis of panels and glycoprotein or glycosite sets as described herein and individual components thereof for certain ethnic populations, genders, etc. and for analysis over a lifetime for individuals based upon the data from millions or more individuals. In addition, the present invention contemplates the storage an access to such information via an appropriate secured and private setting wherein HIPAA standards are followed.

Another aspect of the invention relates to a method for conducting a business, which includes: (a) manufacturing one or more of the detection reagents, panels, arrays, (b) providing services for analyzing tissue-derived serum glycoprotein molecular blood fingerprints and (c) marketing to healthcare providers the benefits of using the detection reagents, panels, arrays, and services of the present invention to enhance capabilities to detect disease or disease progression and thus, to better treat patients.

Another aspect of the invention relates to a method for conducting a business, comprising: (a) providing a distribution network for selling the detection reagents, panels, arrays, diagnostic services, and access to glycoprotein or glycosite molecular blood fingerprint databases (b) providing instruction material to physicians or other skilled artisans for using the detection reagents, panels, arrays, and blood fingerprint databases to improve the ability to detect disease, analyze disease progression, or stratify patients.

For instance, the subject business methods can include an additional step of providing a sales group for marketing the database, or panels, or arrays, to healthcare providers.

Another aspect of the invention relates to a method for conducting a business, comprising: (a) preparing one or more normal tissue- and/or serum-derived glycoprotein or glycosite fingerprints and (b) licensing, to a third party, the rights for further development and sale of panels, arrays, and information databases related to the fingerprints of (a).

The business methods of the present application relate to the commercial and other uses, of the methodologies, panels, arrays, glycoproteins or glycosites (e.g., including the glycoproteins and glycosited described in Table 1 and diagnostic/prognostic panels thereof), blood fingerprints, and databases comprising identified fingerprints of the present invention. In one aspect, the business method includes the marketing, sale, or licensing of the present invention in the context of providing consumers, i.e., patients, medical practitioners, medical service providers, and pharmaceutical distributors and manufacturers, with all aspects of the invention described herein, (e.g., the methods for identifying tissue-derived and/or serum-derived glycoproteins, detection reagents for such proteins, molecular blood fingerprints, etc., as provided by the present invention).

In a particular embodiment of the present invention, a business method or diagnostic method relating to providing expression information related to the glycoproteins and glycosites described herein, or transcripts encoding such glycoproteins or glycosites, a plurality thereof, or a fingerprint of a plurality (e.g., levels of the glycoproteins that make up a given fingerprint), method of determining same or levels thereof or fingerprints of the same and sale of panels comprising same. In a specific embodiment, that method may be implemented through the computer systems of the present invention. For example, a user (e.g. a health practitioner such as a physician or a diagnostic laboratory technician) may access the computer systems of the present invention via a computer terminal and through the Internet or other means. The connection between the user and the computer system is preferably secure.

In practice, the user may input, for example, information relating to a patient such as the patient“s disease state and/or drugs that the patient is taking, e.g., levels determined for the glycoproteins or glycosites of interest or that make up a given molecular blood fingerprint using a panel or array of the present invention. The computer system may then, through the use of the resident computer programs, provide a diagnosis, detect changes in disease states, stratify patients, or determination of drug side-effects that fits with the input information by matching the parameters of (e.g., expression levels of) particular glycoprotein, glycosite or panel thereof with a database of fingerprints.

A computer system in accordance with a preferred embodiment of the present invention may be, for example, an enhanced IBM AS/400 mid-range computer system. However, those skilled in the art will appreciate that the methods and apparatus of the present invention apply equally to any computer system, regardless of whether the computer system is a complicated multi-user computing apparatus or a single user device such as a personal computer or workstation. Computer systems suitably comprise a processor, main memory, a memory controller, an auxiliary storage interface, and a terminal interface, all of which are interconnected via a system bus. Note that various modifications, additions, or deletions may be made to the computer system within the scope of the present invention such as the addition of cache memory or other peripheral devices.

The processor performs computation and control functions of the computer system, and comprises a suitable central processing unit (CPU). The processor may comprise a single integrated circuit, such as a microprocessor, or may comprise any suitable number of integrated circuit devices and/or circuit boards working in cooperation to accomplish the functions of a processor.

In a preferred embodiment, the auxiliary storage interface allows the computer system to store and retrieve information from auxiliary storage devices, such as magnetic disk (e.g., hard disks or floppy diskettes) or optical storage devices (e.g., CD-ROM). One suitable storage device is a direct access storage device (DASD). A DASD may be a floppy disk drive that may read programs and data from a floppy disk. It is important to note that while the present invention has been (and will continue to be) described in the context of a fully functional computer system, those skilled in the art will appreciate that the mechanisms of the present invention are capable of being distributed as a program product in a variety of forms, and that the present invention applies equally regardless of the particular type of signal bearing media to actually carry out the distribution. Examples of signal bearing media include: recordable type media such as floppy disks and CD ROMS, and transmission type media such as digital and analog communication links, including wireless communication links.

The computer systems of the present invention may also comprise a memory controller, through use of a separate processor, which is responsible for moving requested information from the main memory and/or through the auxiliary storage interface to the main processor. While for the purposes of explanation, the memory controller is described as a separate entity, those skilled in the art understand that, in practice, portions of the function provided by the memory controller may actually reside in the circuitry associated with the main processor, main memory, and/or the auxiliary storage interface.

Furthermore, the computer systems of the present invention may comprise a terminal interface that allows system administrators and computer programmers to communicate with the computer system, normally through programmable workstations. It should be understood that the present invention applies equally to computer systems having multiple processors and multiple system buses. Similarly, although the system bus of the preferred embodiment is a typical hardwired, multidrop bus, any connection means that supports bidirectional communication in a computer-related environment could be used.

The main memory of the computer systems of the present invention suitably contains one or more computer programs relating to the molecular blood fingerprints and an operating system. Computer program is used in its broadest sense, and includes any and all forms of computer programs, including source code, intermediate code, machine code, and any other representation of a computer program. The term “memory” as used herein refers to any storage location in the virtual memory space of the system. It should be understood that portions of the computer program and operating system may be loaded into an instruction cache for the main processor to execute, while other files may well be stored on magnetic or optical disk storage devices. In addition, it is to be understood that the main memory may comprise disparate memory locations.

As should be clear to the skilled artisan from the above, the present invention provides databases, readable media with executable code, and computer systems containing information comprising predetermined normal serum levels of glycoprotein and glycosites sets as described herein. Further, the present invention provides databases of information comprising disease-associated fingerprints as well as panels and in some embodiments, levels thereof.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Further, the following examples are offered by way of illustration, and not by way of limitation.

### EXAMPLES

### Example 1

This Example demonstrates that tissue-derived proteins are both present and detectable in plasma via direct mass spectrometric analysis of captured glycopeptides, and thus provides a conceptual basis for plasma protein biomarker discovery and analysis. Further, this Example provides tissue-derived proteins detectable in plasma that have utility in a variety of diagnostic settings.

Materials and Reagents

For chromatography procedures, HPLC-grade reagents from Fisher Scientific (Pittsburgh, Pa.) were used. PNGase F was purchased from New England Biolabs (Beverly, Mass.) and hydrazide resin from Bio-Rad (Hercules, Calif.). All other chemicals used in this study were purchased from Sigma (St. Louis, Mo.). The SK-BR-3, Ramos, and Jurkat cells were obtained from ATCC (American Type Culture Collection, Manassas, Va.). Human tissue specimens were obtained from organs surgically removed because of cancer under a human subject approval for prostate and bladder cancer biomarker discovery project supported by the Early Detection Research Network from the National Cancer Institute.

Purification and Fractionation of N-Linked Glycopeptides from Plasma

The N-linked glycosites identified from plasma were generated from data from four separate resources of human serum or plasma. Two of the plasma samples were from a study performed as part of the HUPO plasma proteome project (Omenn G S, States D J, Adamski M, et al. (2005) Proteomics 5: 3226-3245). One of these HUPO plasma samples was an equal mix (v/v) of plasma from one male and one post-menopausal female Caucasian-American donors. These samples were collected with sodium citrate as anticoagulant (BD Diagnostics). The second HUPO plasma sample was from the UK National Institute of Biological Standards and Control (NIBSC) provided as a lyophilized citrated plasma standard from a pool of 25 donors (Omenn G S, States D J, Adamski M, et al. (2005) Proteomics 5: 3226-3245). The third sample source for this study was generated at the Institute for Systems Biology (ISB) from a pool of serum samples collected from 7 healthy male donors and 3 healthy female donors. Following approval by Human Subject Institutional Review Board of ISB, trained phlebotomists collected blood from each donor into evacuated blood collection tubes. Blood was allowed to clot for 1 hr at room temperature. Sera were collected by centrifugation at 3000 rpm. It should be noted that using these collection procedures for plasma and serum samples, contamination from breakage of platelet or other blood cells cannot be totally ruled out. Formerly N-linked glycosylated peptides were isolated using N-linked glycopeptide capture procedure as described previously (Zhang H, Li X J, Martin D B, Aebersold R. (2003) Identification and quantification of N-linked glycoproteins using hydrazide chemistry, stable isotope labeling and mass spectrometry. Nat Biotechnol 21: 660-666; Desiere F, Deutsch E W, Nesvizhskii A I, et al. (2005) Integration with the human genome of peptide sequences obtained by high-throughput mass spectrometry. Genome Biol 6: R9; Deutsch E W, Eng J K, Zhang H, et al. (2005) Human Plasma PeptideAtlas. Proteomics 5: 3497-3500). For these studies, 750 μl of serum or plasma was used for N-linked glycopeptide isolation. The fourth set of data used for this study was generated from a previously published study of N-linked plasma glycopeptides from Biological Systems Analysis and Mass Spectrometry group at Pacific Northwest National Laboratory (PNNL) in Richland, Wash. (Liu T, Qian W J, Gritsenko M A, et al. (2005) Human plasma N-glycoproteome analysis by immunoaffinity subtraction, hydrazide chemistry, and mass spectrometry. J Proteome Res 4: 2070-2080).

Purification and Fractionation of N-Linked Glycopeptides from Cells and Solid Tissues

Proteins from SK-BR-3 breast cancer cells were extracted via homogenization and fractionation of cell lysates. At confluence, SK-BR-3 cells were rinsed 5 times with serum-free medium, followed by incubation in serum-free McCoy's 5a for 24 h at 37° C. in a humidified incubator at 5% CO₂. Cells were homogenized in 0.32M sucrose, 100 mM sodium phosphate, pH7.5, and separated into three fractions by sequential centrifugations (1,000×g pellet, 17,000×g pellet, and 17,000×g supernatant) (Han D K, Eng J, Zhou H, Aebersold R. (2001) Quantitative profiling of differentiation-induced microsomal proteins using isotope-coded affinity tags and mass spectrometry. Nat Biotechnol 19: 946-951). Protein extraction from solid tissues was performed using cell-free supernatant after an initial digestion of the tissues with collagenase. The tissues was sliced into pieces in serum-free cell culture medium and collagenase was added at a final concentration of 1 mg/ml. Tissues were digested overnight at room temperature with stirring and a cell-free supernatant was obtained by centrifugation (Liu A Y, Zhang H, Sorensen C M, Diamond D L. (2005) Analysis of prostate cancer by proteomics using tissue specimens. J Urol 173: 73-78; Zhang H, Li X J, Martin D B, Aebersold R. (2003) Identification and quantification of N-linked glycoproteins using hydrazide chemistry, stable isotope labeling and mass spectrometry. Nat Biotechnol 21: 660-666). One mg aliquots of protein extracted from cultured breast cells and solid tissue samples was used for glycopeptide capture (Zhang H, Li X J, Martin D B, Aebersold R. (2003) Identification and quantification of N-linked glycoproteins using hydrazide chemistry, stable isotope labeling and mass spectrometry. Nat Biotechnol 21: 660-666).

Isolation of glycopeptides from the plasma membrane of lymphocytes was by a modification of the glycopeptide-capture method (Zhang H, Li X J, Martin D B, Aebersold R. (2003) Identification and quantification of N-linked glycoproteins using hydrazide chemistry, stable isotope labeling and mass spectrometry. Nat Biotechnol 21: 660-666) that allows for specific labeling/isolation of just plasma membrane glycoproteins (Wollscheid et al. manuscript in preparation). In brief, this was accomplished by the use of a biotinylated hydrazide instead of a solid-phase hydrazide to label only the cell surface glycoproteins on live B and T lymphocytes in culture. After labeling, total membrane proteins were again isolated from the cells (Han D K, Eng J, Zhou H, Aebersold R. (2001) Quantitative profiling of differentiation-induced microsomal proteins using isotope-coded affinity tags and mass spectrometry. Nat Biotechnol 19: 946-951) which were then proteolyzed with trypsin. Capture of plasma membrane-derived biotinylated glycopeptides was achieved via streptavidin-affinity isolation (Gygi S P, Rist B, Gerber S A, Turecek F, Gelb M H, Aebersold R. (1999) Quantitative analysis of complex protein mixtures using isotope-coded affinity tags. Nat Biotechnol 17: 994-999), and the N-linked glycopeptides once again recovered following cleavage with PNGase F.

Analysis of Peptides by Mass Spectrometry

Off-line fractionation of peptides isolated from human plasma samples by strong cation-exchange chromatography prior to analysis of each fraction via LC-MS/MS was performed as described previously (Han D K, Eng J, Zhou H, Aebersold R. (2001) Quantitative profiling of differentiation-induced microsomal proteins using isotope-coded affinity tags and mass spectrometry. Nat Biotechnol 19: 946-951). Peptides from other sources were analyzed by online reverse phase LC-MS/MS without further sample fractionation.

Fractionated peptides from plasma samples were analyzed using both an LCQ and LTQ ion-trap mass spectrometer (Thermo Finnigan, San Jose, Calif.) as well as with electrospray ionization quadrupole-time-of-flight (ESI-qTOF) mass spectrometer (Waters, Milford, Mass.) according to standard practices and manufacturers' instructions (Zhang H, Yi E C, Li X J, et al. (2005) High throughput quantitative analysis of serum proteins using glycopeptide capture and liquid chromatography mass spectrometry. Mol Cell Proteomics 4: 144-155).

Peptides isolated from solid tissues and breast cancer cells were identified using an LCQ or LTQ ion trap mass spectrometer. The peptides were injected in three aliquots into a homemade peptide cartridge packed with Magic C18 (Michrom Bioresources, Auburn, Calif.) using a FAMOS autosampler (DIONEX, Sunnyvale, Calif.), and then passed through a 10 cm×75 μm i.d. microcapillary HPLC column packed with Magic C18 resin. A linear gradient of acetonitrile from 5%-32% over 100 min at a flow rate of ˜300 nl/min was applied. MS/MS spectra were acquired in a data-dependent mode.

Peptides isolated from B and T lymphocyte plasma membranes were analyzed on an LCQ ion trap mass spectrometer as previously described (Gygi S P, Rist B, Gerber S A, Turecek F, Gelb M H, Aebersold R. (1999) Quantitative analysis of complex protein mixtures using isotope-coded affinity tags. Nat Biotechnol 17: 994-999).

Acquired MS/MS spectra were searched against the International Protein Index (IPI) human protein database (version 2.28, containing 40,110 entries) using SEQUEST software (Eng J, McCormack A L, Yates J R, 3rd. (1994) An approach to correlate tandem mass spectral data of peptides with amino acid sequences in a protein database. J. Am. Soc. Mass Spectrom. 5: 976-989). The database search parameters were set to the following modifications: carboxymethylated cysteines, oxidized methionines, and a (PNGase F-catalyzed) conversion of Asn to Asp that occurs at the original site of carbohydrate attachment to the peptide/protein (i.e the N-glycosite). No other constraints were included for database searches.

Database search results were then statistically analyzed using PeptideProphet, which effectively computes a probability for the likelihood of each identification being correct (on a scale of 0 to 1) in a data-dependent fashion (Keller A, Nesvizhskii A I, Kolker E, Aebersold R. (2002) Empirical statistical model to estimate the accuracy of peptide identifications made by MS/MS and database search. Anal Chem 74: 5383-5392). A PeptideProphet probability score of ≧0.9 was used as a filter to remove low probability peptides identifications. This filtering step represented an estimated peptide sequence assignment error rate of 2% or less for all datasets as calculated by PeptideProphet. Although the majority of N-linked glycosylation occurs at a consensus N—X—S/T sequon (where X is any amino acid except proline) (Bause E. (1983) Structural requirements of N-glycosylation of proteins. Studies with proline peptides as conformational probes. Biochem J 209: 331-336.), ˜20% of identified peptides did not contain such a sequon. These peptide identifications likely resulted from false positive identifications from the database search, non-specific isolation of N-linked glycosites, and from the isolation of atypical N-linked glycosites (i.e., not containing the N—X—S/T motif) of which we do not have sufficient understanding to predict. Thus, to reduce the false positive rate of the identified N-linked glycosites and to focus on those N-linked glycosites we could be most confident about, the peptide sequences were additionally filtered to remove non-motif-containing peptides. Finally, peptide sequences were analyzed with respect to individual unique N—X—S/T sequons such that overlapping sequences containing the same N—X—S/T sequon (i.e. redundant N-linked glycopeptides for the same N-linked glycosite) were resolved in favor of those peptide sequences that contained the greater number of tryptic cleavage termini.

Sub-Cellular Localization of Identified Proteins

In order to predict the likely sub-cellular localization of identified peptides/proteins, we utilized freely available prediction software for determination of (secretion) signal peptides and likely cell membrane-spanning sequences. Signal peptides were predicted using SignalP 2.0 (Nielsen H, Engelbrecht J, Brunak S, von Heijne G. (1997) A neural network method for identification of prokaryotic and eukaryotic signal peptides and prediction of their cleavage sites. Int J Neural Syst 8: 581-599) and transmembrane (TM) regions were predicted using TMHMM (version 2.0) (Krogh A, Larsson B, von Heijne G, Sonnhammer E L. (2001) Predicting transmembrane protein topology with a hidden Markov model: application to complete genomes. J Mol Biol 305: 567-580) for protein topology and the number of TM helices. Information from both SignalP and TMHMM were combined to allow for sorting of the identified N-glycosylated proteins into the following categories: i) cell surface—proteins that contained predicted non-cleavable signal peptides and no predicted TM segments; ii) secreted—proteins that contained predicted cleavable signal peptides and no predicted TM segments; iii) transmembrane—proteins that contained predicted TM segments and extracellular loops and intracellular loops; and iv) intracellular—proteins that contained neither predicted signal peptides nor predicted TM segments.

Results:

The goal of this study was to test whether bona fide peptides derived from a variety of cell or tissue types were also detectable in blood plasma and to identify tissue-derived serum glycoproteins for use in diagnostic panels. Since cell surface and secreted proteins are both likely to be deposited into the blood and most of them are also glycosylated, the glycoprotein sub-proteome that could be readily identified from both selected cultured cell lines and solid tumor samples was targeted. It was then determined whether a significant subset of these cell- and tissue-derived glycoproteins were indeed similarly detectable and thus present in blood plasma.

The general approach employed for these analyses is summarized in FIG. 1 and consists of four basic steps: 1) Protein extraction. Proteins were extracted from cells via homogenization and differential centrifugations (Han D K, Eng J, Zhou H, Aebersold R. (2001) Quantitative profiling of differentiation-induced microsomal proteins using isotope-coded affinity tags and mass spectrometry. Nat Biotechnol 19: 946-951). For protein extraction from solid tissues, tissues were digested with collagenase to obtain a cell-free supernatant (Liu A Y, Zhang H, Sorensen C M, Diamond D L. (2005) Analysis of prostate cancer by proteomics using tissue specimens. J Urol 173: 73-78.). 2) Glycopeptide capture. Proteins from tissues/cells and plasma were processed by the recently described solid-phase-based method for the isolation of N-linked glycopeptides (Zhang H, Li X J, Martin D B, Aebersold R. (2003) Identification and quantification of N-linked glycoproteins using hydrazide chemistry, stable isotope labeling and mass spectrometry. Nat Biotechnol 21: 660-666.). The end-product for this procedure is the isolation of de-glycosylated peptides that originally contain N-linked carbohydrates in the native protein (Zhang H, Li X J, Martin D B, Aebersold R. (2003) Identification and quantification of N-linked glycoproteins using hydrazide chemistry, stable isotope labeling and mass spectrometry. Nat Biotechnol 21: 660-666). This also results in the conversion of the formerly glycosylated Asn to an Asp side chain. 3) Peptide identification. Isolated peptides were analyzed by automated LC-MS/MS. SEQUEST database search was performed for peptide sequence identification (Eng J, McCormack A L, Yates J R, 3rd. (1994) An approach to correlate tandem mass spectral data of peptides with amino acid sequences in a protein database. J. Am. Soc. Mass Spectrom. 5: 976-989) followed by implementation of PeptideProphet (Keller A, Nesvizhskii A I, Kolker E, Aebersold R. (2002) Empirical statistical model to estimate the accuracy of peptide identifications made by MS/MS and database search. Anal Chem 74: 5383-5392) for statistical determination of the peptide identifications most likely to be correct. 4) Peptide comparison. Peptides identified from the different samples were compared against each other to determine the peptides in common between different cell- and tissue-types, as well as to peptides identified from plasma to determine which cell/tissue-derived proteins/peptides were also detectable in plasma (see Table 1).

Table 1 associated with this application is provided on CD-ROM in lieu of a paper copy, and is hereby incorporated by reference into the specification. Identified peptide sequences were first assigned to proteins in the IPI database (version 2.28). Assigned proteins were then mapped to RNA sequences in the RefSeq database (NCBI build number 36) using connections stored in the IPI database and in EntrezGene database (modified on Sep. 18, 2006).

The legend to Table 1 is outlined below:

TABLE 1A
Legend

| Column Header | Information contained in the column |
|---|---|
| PP | Peptide Prophet Score |
| BLCT | Bladder Cancer Tissue |
| BRCC | Breast Cancer Cell |
| BRCT | Breast Cancer Tissue |
| LCT | Liver Cancer Tissue |
| LY | Lymphocyte |
| OCC | Ovarian Cancer Cell |
| OCT | Ovarian Cancer Tissue |
| PCC | Prostate Cancer Cell |
| PCT | Prostate Cancer Tissue |
| PL | Plasma |
| GlyID | Identified Glycosite SEQ ID NO |
| Glycosite | Identified Glycosite amino acid sequence |

TABLE 1B
Legend

| Column Header | Information contained in the column |
|---|---|
| GlyID | Identified Glycosite SEQ ID NO |
| IPI Access | IPI Accession Number |
| PRSEQID | Protein Sequence SEQ ID NO |
| Prot Descr | Protein Description (from IPI) |
| Prot Loc | Protein Localization |
| REFSEQAcc | RefSeq Acession Number for the mapped |
| | nucleic acid sequence |
| PNSEQID | RefSeq Polynucleotide SEQ ID NO: |

Since the general isolation procedures used here specifically targeted N-linked glycosylation and since there is a known consensus sequence for this modification (N—X—S/T, X can be any amino acid except P), the comparisons were limited solely to the identified peptide sequences that contained at least one such N-linked glycosylation motif in order to simplify and to further reduce false positive rates.

Glycoproteins expressed on the surface of two human lymphocyte cell lines were characterized, one of B cell and one of T cell lineage (Ramos and Jurkat, respectively). Since lymphocytes naturally circulate in the blood, they come in contact with the blood plasma as much or more than any other cell type, thus maximizing the likelihood of their proteins being deposited into the plasma.

N-linked glycopeptides were isolated and identified from the plasma membranes of both Jurkat and Ramos cells for comparison to a previously compiled list of identified N-linked glycosites derived from plasma glycoproteins (Desiere F, Deutsch E W, Nesvizhskii A I, et al. (2005) Integration with the human genome of peptide sequences obtained by high-throughput mass spectrometry. Genome Biol 6: R9; Deutsch E W, Eng J K, Zhang H, et al. (2005) Human Plasma PeptideAtlas. Proteomics 5: 3497-3500; Liu T, Qian W J, Gritsenko M A, et al. (2005) Human plasma N-glycoproteome analysis by immunoaffinity subtraction, hydrazide chemistry, and mass spectrometry. J Proteome Res 4: 2070-2080). A total of 384 N-linked glycosites from B and T cell-surface glycoproteins were identified with a PeptideProphet score of ≧0.9. When compared with previously compiled data on 1105 identified N-linked glycosites from plasma proteins (similarly scoring ≧0.9 with PeptideProphet), 77 of the N-linked glycosites were in common with those already identified from plasma (FIG. 2 and Table 1). This represented a significant portion (20%) of the total identifications from the B and T lymphocyte cell plasma membranes, thus confirming that lymphocyte-derived glycoproteins are both present and readily detectable in plasma when using this fairly simple glycoprotein/glycopeptide enrichment protocol upstream of identification by LC-MS/MS.

Since these identifications were achieved using cells grown in culture media supplemented with bovine serum, there was no potential for human blood contamination for these samples. However, some identifications could be attributed to bovine proteins should there be sufficient sequence homologies with human. To investigate this possibility, the sequences of the 77 N-linked glycosites representing this lymphocyte/plasma overlap were submitted to a search of the bovine protein database (internet address: bovine dot nci dot 20051213). These results indicated that only 10 of the 77 N-linked glycosites were conserved between human and bovine. For these 10 N-linked glycosites, the source of origin could not be reliably assigned. However, for the remaining 67 N-linked glycosites that were not conserved, it can be concluded that they could only have originated from the human cells under study, thus indicating that most or all of the plasma membrane glycoproteins identified from the human lymphocytes originated from the cells themselves rather than the culture medium. Thus, these data combined clearly indicated that glycoproteins expressed on the surface of lymphocytes were indeed detectable in the blood via solid-phase based isolation and LC-MS analysis of N-linked glycopeptides.

Since blood cells such as B and T lymphocytes and platelets naturally circulate in the blood, it was also possible that proteins could have been artificially introduced from such cells into the plasma during the blood/plasma collection rather than by natural release into the blood in vivo. While this eventuality was difficult to experimentally exclude completely during the serum/plasma collection process, a clue as to whether this was generally a problem might be inferable from microarray data. To this end, proteins identified in both prostate and plasma in this study were compared with the transcriptional profiling data of these proteins in whole blood from available published microarray analyses (Nielsen H, Engelbrecht J, Brunak S, von Heijne G. (1997) A neural network method for identification of prokaryotic and eukaryotic signal peptides and prediction of their cleavage sites. Int J Neural Syst 8: 581-599.; Su A I, Cooke M P, Ching K A, et al. (2002) Large-scale analysis of the human and mouse transcriptomes. Proc Natl Acad Sci USA 99: 4465-4470). Transcription data was found for 162 out of 202 N-linked glycosites that were identified in both prostate tissue and plasma (FIG. 2 and Table 1), of which 78 were not detected in blood cells (an average difference value of 200 was used as threshold to make present/absent calls (Su A I, Cooke M P, Ching K A, et al. (2002) Large-scale analysis of the human and mouse transcriptomes. Proc Natl Acad Sci USA 99: 4465-4470). For 84 N-linked glycosites that were shown to be present in blood cells, genes for 20 N-linked glycosites were highly expressed in blood cells (expression in blood cells was 5-fold of the median value for 64 tissues or cells used). Therefore, the tissue origin of these 20 N-linked glycosites can not be determined. On the other hand, a number of N-linked glycosites identified in both prostate tissue and plasma were preferentially expressed in prostate tissue but not in blood cells shown by microarray analyses. These included CD26, lumican, MAC-2 binding protein, basement membrane-specific heparan sulfate proteoglycan core protein, and desmoglein (Table 1). These observations suggest that the majority of proteins that were detected in both tissues and plasma were likely deposited into the plasma from tissues in vivo.

Next, it was tested whether the observation of such an overlap between N-linked glycosites identified from both lymphocytes and blood plasma could be extended to other cell types and tissues whose cells do not circulate in the blood stream. For this, four different but representative cell/tissue types pertinent to cancer biomarker discovery were selected to determine whether the N-linked glycosites identifiable from these sources are also present in the larger plasma dataset. Specifically, we chose SK-BR-3 breast cancer cells, primary bladder and prostate cancer tissue, and a liver metastasis of prostate cancer.

N-linked glycopeptides from the cultured SK-BR-3 breast cancer cells were isolated from a whole-cell lysate via conventional solid-phase glycoprotein/glycopeptide enrichment method. Similarly, hydrazide-based isolation of N-linked glycopeptides from tissues was carried out with cell-free supernatants of collagenase-digested prostate, bladder, and liver metastasis tissue specimens (FIG. 1) (Zhang H, Li X J, Martin D B, Aebersold R. (2003) Identification and quantification of N-linked glycoproteins using hydrazide chemistry, stable isotope labeling and mass spectrometry. Nat Biotechnol 21: 660-666; Liu A Y, Zhang H, Sorensen C M, Diamond D L. (2005) Analysis of prostate cancer by proteomics using tissue specimens. J Urol 173: 73-78). The identification of isolated N-linked glycopeptides was via LC-MS/MS and the results similarly compared with the plasma dataset (Zhang H, Loriaux P, Eng J, et al. (2006) UniPep, a database for human N-linked glycosites: A Resource for Biomarker Discovery. Genome Bio 7: R73). When combined with the lymphocyte data, these data showed that of the total 1,257 N-linked glycosites identified in the two cell and three tissue types, 832 of these were identified in only one of the sample types (Table 1). FIG. 2 summarizes the total number of N-linked glycosites identified in each cell/tissue type, the number of these that were unique to each specific cell or tissue type, as well as the subsets of these that additionally overlapped with the plasma-derived N-liked glycosite dataset.

Similar to the comparison between lymphocytes and plasma, all four of these additional datasets showed a significant overlap with the plasma dataset. As can be seen from FIG. 2, some of the N-linked glycosites identified in both a particular cell/tissue and plasma were unique to that cell/tissue type. For example, of the 286 N-linked glycosites in common between plasma and breast cancer cells, 123 were not identified in any of the other cell/tissue samples evaluated. These results again support the contention that glycoproteins originating from cells or tissues are detectable in plasma using the relatively simple methodological approach of LC-MS analysis of enriched N-linked glycoproteins. Furthermore, they indicate that glycoproteins from all or most cell and tissue types are likely to be found in the blood and be present at detectable levels for such an analytic approach.

In the above studies, proteins were identified by LC-MS/MS. In this method, not all proteins from cells, tissues or plasma are identified due to the random sampling of peptide precursor ions during the analytical process. Therefore, we focused this study on the proteins commonly detected in both cell/tissue and plasma, and put less value on the proteins only detected in specific tissues (tissue specificity). In addition, tumor cells and tissues were used to isolate the cell/tissue N-linked glycopeptides whereas the dataset for plasma proteins was derived from samples obtained from non-cancer patient donors. Therefore, without quantitative comparison of protein concentration in normal and cancer plasma, we cannot confirm that the N-linked glycosites identified in common between tissues/cells and plasma shown here are associated with cancer. Conversely, N-linked glycosites identified from cancer cells/tissues but not detected in the current plasma dataset could be potential cancer biomarkers for detection in plasma of cancer patients. For example, two prostate cancer tissue proteins, prostatic acid phosphatase (PAP) and prostate-specific antigen (PSA) were not found in the plasma dataset. The levels of these proteins have been shown to be elevated in the plasma of prostate cancer patients and are unlikely to be detected in plasma of normal donors (Ludwig J A, Weinstein J N. (2005) Biomarkers in cancer staging, prognosis and treatment selection. Nat Rev Cancer 5: 845-856).

Unlike cultured cells, tissues are vascularized. One would thus expect that some contamination of the tissue glycoproteins by common circulating blood glycoproteins would inevitably occur. To investigate this possibility, the cell/tissue-derived data was examined to see if the overlap of N-linked glycosites detected in both plasma and the respective tissue sources could be explained by simple contamination from blood proteins. If this were the case, then it would be expected that such contaminating plasma-derived glycoproteins would be a general effect and thus be detected in multiple tissues.

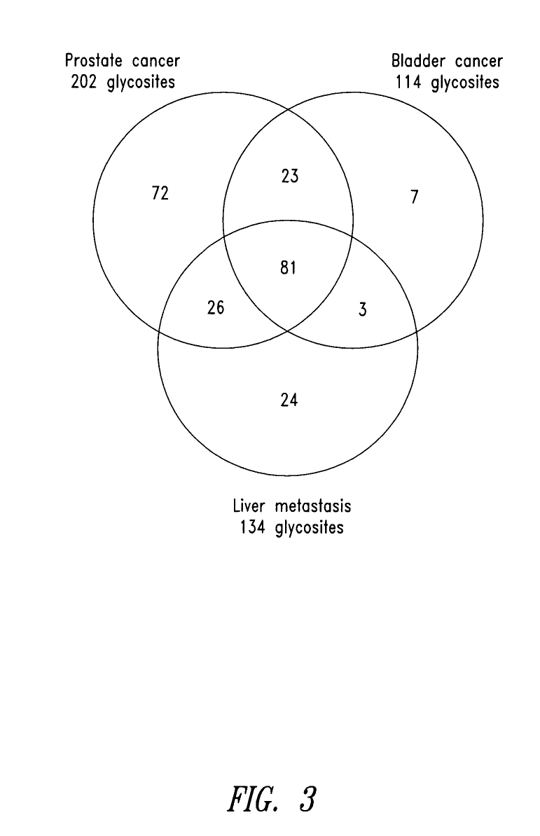

When this comparison was made, it was found that a significant number identified N-linked glycosites were indeed common to multiple tissues (FIG. 3 and Table 1). For example, 202 unique N-linked glycosites were identified in both prostate tissue and plasma. By referencing available database annotations for these proteins, it was determined that 94 of these N-linked glycosites likely originated from proteins made by prostatic cells, with another 96 to originate from blood. The remaining 12 N-linked glycosites were annotated as hypothetical proteins whose origin could not be determined. Furthermore, when the N-linked glycosites identified were compared from both prostate cancer tissue and plasma with the N-linked glycosites identified from the other two tissues (bladder cancer and liver metastasis) and plasma, it was found that 81 of the N-linked glycosites identified were shared among all 3 tissues. Of these, 57 (70%) were annotated as classical plasma proteins (FIG. 3, Table 1). In contrast, it would be expected that the peptides identified from only one of these tissues would be far more likely to represent bona fide tissue-derived proteins. Indeed, for the 129 N-linked glycosites that were uniquely identified in prostate cancer tissue, it was found that only 7 N-linked glycosites (5%) were annotated as classical plasma proteins. These observations again suggested that this technique enabled the identification of significant numbers of genuine tissue-derived glycoproteins in both tissue and plasma samples, without being overwhelmed by high abundance plasma proteins.

The initial premise for specifically targeting N-linked glycosites in this study was two-fold. First, the reduction in sample complexity achieved by selectively focusing on the sub-proteome of N-linked glycopeptides was expected to improve the detection sensitivity in mass spectrometric analysis of the resulting sample mixtures. Second, the vast majority of intracellular proteins are non-glycosylated, whereas a significant proportion of plasma membrane-bound, extracellular and secreted proteins, including plasma proteins, are glycosylated. Thus glycoproteins should represent an ideal class of proteins to target for the discovery of new markers of disease that are detectable and quantifiable in the blood.

To test whether sampling did indeed include these expected categories of proteins in our analyses, an informatics approach was applied for the prediction of likely sub-cellular localization for the glycoproteins identified in the various tissues and cells studied, classifying them into four general groups: 1) cell surface proteins, 2) secreted proteins, 3) transmembrane proteins and 4) intracellular proteins. Glycoproteins would be expected to fall into one of the first 3 of these groups and, not surprisingly, this analyses confirmed that 1168 out of a total of 1257 (93%) N-linked glycosites identified from tissues, cells, or plasma were classified as such (see Table 1). Indeed, the true percentage of such proteins in this dataset was likely even higher than 93% since some of the N-linked glycosites predicted as intracellular proteins were in fact immunoglobulin isoforms, proteins known to be secreted in actuality. In contrast, applying the same informatic methodology to all 40,110 entries in the human protein sequence database that was used for searching the MS/MS data showed that about a third of proteins in the database could be similarly classified (data not shown). These observations thus confirmed the initial premise that the targeted isolation and identification of N-linked glycoproteins and glycopeptides significantly enriched for the desired secreted, extracellular and cell membrane proteins, i.e., proteins that likely represent good candidates for both markers of disease and their quantification in the blood. To further reduce the false positive identification of N-linked glycosites, the protein subcellular location for the identified N-linked glycosites can be further used as a filter to remove the N-linked glycosites from intracellular proteins.

Another largely unanswered question relating to blood biomarker discovery was whether the simple, robust and affordable methodologies required for the necessary high throughput screens were able to access the lower abundance proteins that are generally assumed to be of greater significance for predictive or diagnostic purposes. The data presented here also indicated that by targeting the identification of N-linked glycosites, enabled access to lower-abundance plasma proteins that also might have originated from specific tissues. A representative list of such proteins is shown in FIG. 4 (see also Table 1), including 217 N-linked glycosites from cluster designation (CD) cell surface antigens. Of these, 56 N-linked glycosites from CD antigens were also identified from plasma samples, and 140 of the N-linked glycosites from CD antigens were identified from lymphocyte membranes (Table 1). This high proportion of detection in lymphocytes was to be expected since CD antigens were originally characterized as white blood cell surface proteins (True L D, Liu A Y. (2003) A challenge for the diagnostic immunohistopathologist. Adding the CD phenotypes to our diagnostic toolbox. Am J Clin Pathol 120: 13-15), many of which are now used routinely for typing lymphocytes. However, the expression of many CD antigens is not restricted only to lymphocytes, or cells of the hematopoietic system. In this study, 77 N-linked glycosites from CD antigens were also identified in tissues or cells other than lymphocytes (Table 1). Since the expression of some CD antigens on cancer cells has been shown to differ from their normal counterparts, cancer-specific CD antigens found in plasma might also serve as markers for the detection of cancer of specific tissues (Liu A Y, Roudier M P, True L D. (2004) Heterogeneity in primary and metastatic prostate cancer as defined by cell surface CD profile. Am J Pathol 165: 1543-1556). To confirm that these N-linked glycosites from CD antigens identified from tissues were in fact derived from the tissues themselves rather than via contamination from infiltrating lymphocyte proteins present in the tissues, the available immunohistochemistry (IHC) data for some of these CD molecules were examined, and it was found that in cases where MS identification had been made from a tissue sample, the IHC data were supportive of those findings (FIG. 4).

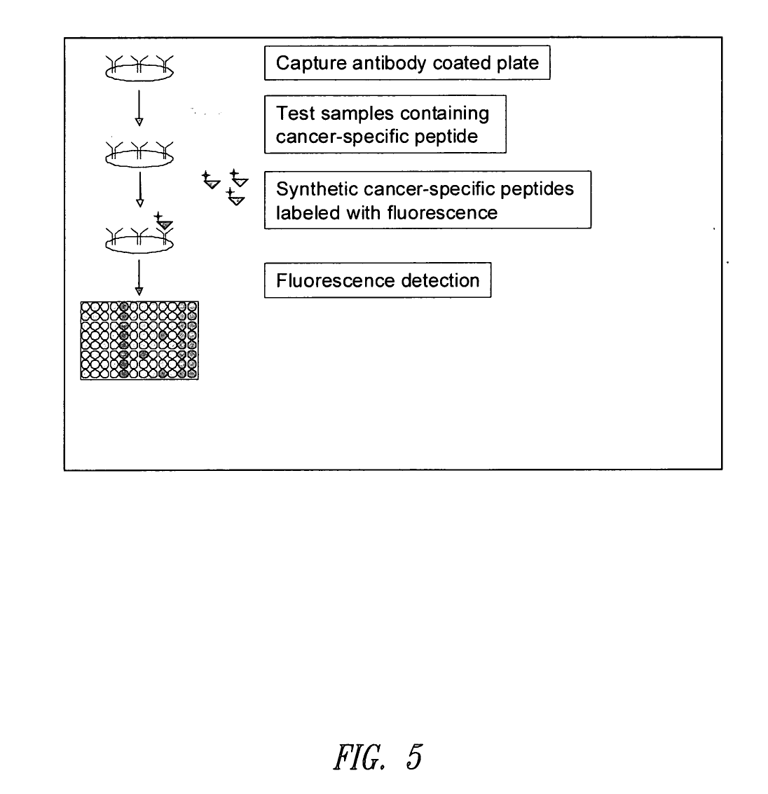

As an additional test of the sensitivity of this approach towards the identification of lower abundance proteins from cells, tissues, and plasma, the N-linked glycosite dataset was compared to recently published literature-derived lists of proteins that have been linked to both cardiac disease and cancer and could thus also represent candidate biomarkers; datasets that also included reported blood concentrations for some of the proteins where also published (Anderson L. (2005) Candidate-based proteomics in the search for biomarkers of cardiovascular disease. J Physiol 563: 23-60; Anderson L, Polanski M. (2006) A list of candidate cancer biomarkers for targeted proteomics. Biomarker Insights In press). When these two published datasets were compared with the N-linked glycosite dataset presented here, it was found that 314 N-linked glycosites were from 141 candidate biomarkers (Table 1). Of these, normal plasma concentrations were also reported for 56 of these proteins. Several of these proteins detected in both cell/tissue and plasma in this study were known to be present in normal plasma at concentrations in the ng/ml to low μg/ml range. Such proteins included prothrombin, tissue inhibitor of metalloproteinase 1, von Willebrand factor, tenascin, L-selectin, CD54 and others (Table 1). FIG. 5 shows a histogram for these known protein concentrations in normal plasma for the proteins we had also detected in both cells/tissues and plasma or cells/tissues alone. As expected, the proteins identified for which normal blood concentrations were also reported were indeed biased towards the more abundant proteins present in the blood. However, these data also showed that despite this, we were nevertheless still able to sample N-glycosylated plasma proteins spanning a wide concentration range spanning at least the top 8 orders of magnitude of the full plasma protein concentration range. From these results, it was concluded that through targeting N-linked glycopeptide enrichment identification via LC-MS/MS, we were able to access the lower abundance tissue- and cell-derived proteins that many believe constitute the richest source of potentially new disease markers.

Thus, through the application of solid-phase glycopeptide enrichment and LC-MS, this method clearly enables detection of cell-surface CD antigens in plasma as well as other molecules known to reflect important physiological information about the state of a particular tissue or cell type. In fact, expression patterns of some CD molecules have already been correlated to disease states of certain tissues, including cancer of the colon, thyroid and prostate (Weichert W, Knosel T, Bellach J, Dietel M, Kristiansen G. (2004) ALCAM/CD166 is overexpressed in colorectal carcinoma and correlates with shortened patient survival. J Clin Pathol 57: 1160-1164; Kholova I, Ryska A, Ludvikova M, Pecen L, Cap J. (2003) [Dipeptidyl peptidase IV (DPP IV, CD 26): a tumor marker in cytologic and histopathologic diagnosis of lesions of the thyroid gland]. Cas Lek Cesk 142: 167-171; Kristiansen G, Pilarsky C, Wissmann C, et al. (2003) ALCAM/CD166 is up-regulated in low-grade prostate cancer and progressively lost in high-grade lesions. Prostate 54: 34-43). Two other proteins identified in this study, the MAC-2 binding protein and metalloproteinase inhibitor 1, have also been identified as potential cancer markers from multiple tissue types, with their quantification in blood being of use in monitoring cancer progression (Marchetti A, Tinari N, Buttitta F, et al. (2002) Expression of 90K (Mac-2 BP) correlates with distant metastasis and predicts survival in stage I non-small cell lung cancer patients. Cancer Res 62: 2535-2539; Liu A Y, Zhang H, Sorensen C M, Diamond D L. (2005) Analysis of prostate cancer by proteomics using tissue specimens. J Urol 173: 73-78).

In a related study, the prostate marker CD90 was further investigated using IHC. The data showed that CD90 is a marker for stromal cells in the prostate. The stromal cells of tumors were stained more intensely than those of benign tissue. This increased CD90 staining appeared to be a common feature for nearly every tumor specimen analyzed. The pronounced CD90 staining could serve to delineate tumor foci, as this staining difference did not appear to extend beyond the tumor area.

While not all the proteins identified from certain tissue/cell are specific to that tissue/cell, this does not preclude them as candidate tissue-specific disease markers, either on their own, or more so as part of a marker panel. In fact, any protein that changes in response to a disease or alteration in physiological state could have value as part of a panel of biomarkers for a specific disease or state, regardless of its ubiquity. Thus taken together, these data suggest that: 1) analyses of glycoproteins from tissue/cell can determine both common and tissue-specific protein profiles for cell surface and secreted proteins from disease tissues; 2) specific cell surface or secreted glycoproteins from tissue/cell are released into circulation at levels detectable by glycopeptide enrichment and MS; 3) certain disease-related changes in the expression patterns of cell surface and secreted proteins from tissue/cell should similarly be detectable in blood.

In conclusion, in this present study, N-linked glycopeptides were isolated from tissues, cells and plasma, and the peptide sequences and proteins that they represent were identified via MS-based proteomics. Glycoproteins identified from the individual tissue and cell types were compared with those identified from plasma. In each case, a significant overlap was observed between the tissue/cell glycoproteins and those observed in plasma. Taken together, these data demonstrate that extracellular glycoproteins originating from tissues and cells are released into the blood at levels that are detectable by MS. They also demonstrate that the use of a single, simple solid-phase based enrichment of glycoproteins/glycopeptides from blood plasma, upstream of LC-MS analysis, is sufficient to allow for measurement and profiling of such tissue-derived and cellular proteins in plasma. Thus this example demonstrated that the largely untested assumption that MS-based proteomic screens are able to detect tissue/cell-derived proteins in the blood is indeed correct, identifed tissue-derived serum glycoproteins useful in a variety of diagnostic settings, and described a methodology capable of accessing such proteins and potential biological and physiological insights they promise.

### Example 2

### Database to Display Identified and Predicted N-Linked Glycopeptides

The large number of N-linked glycopeptides identified in plasma from our study were mapped to all of the theoretical tryptic N-linked glycosylation sequons from the human IPI database (version 2.28). A web interface, UniPep (www dot unipep dot org) was developed to display these theoretical N—X—S/T sequon-containing peptides in the human IPI database along with their corresponding experimentally identified N-linked glycopeptides. This is of particular relevance with respect to those genes or proteins that have been shown to change their abundance in disease tissues compared to normal tissues using either genomic or proteomic approaches. The detection of these proteins in plasma, especially ones that are secreted or expressed on cell surfaces and are therefore most likely to make their way into blood plasma, is a critical step in the development of these proteins as potential disease biomarkers. Gene differential expression analysis has shown that many of the genes up-regulated in ovarian cancer represent surface or secreted proteins such as claudin-3 and -4, HE4, mucin-1, epithelial cellular adhesion molecule, and mesothelin, making surface or secreted proteins from these genes attractive candidate biomarkers that are likely detectable in body fluids (35, 68). In this case, the potential N-linked glycopeptides are selected via UniPep, and heavy isotopic labeled peptides can then be synthesized as standards to determine their presence and to further quantify their abundance in blood.

For each protein in the UniPep database, the database displays three different types of information to allow selection of potential N-linked glycopeptides when scanning the IPI protein database. First, the subcellular location of the protein is predicted. Since N-linked glycosylation is likely to occur in extracellular surface or secreted proteins, we predicted the subcellular localization of each one using a commercial version of the TMHMM algorithm (69), a combination of hidden Markov model (HMM) algorithms (70) and transmembrane (TM) region predictions. By so doing, we were able to categorize each protein as being either extracellular, secreted, transmembrane, or intracellular. The predicted protein subcellular localization is displayed in UniPep along with other protein information from database annotations, and the signal peptides and transmembrane sequences are highlighted in the protein sequence to give a general indication of protein topology. Second, the sequences of all potential N-linked glycopeptides within each protein are displayed as predicted N-linked glycopeptides. For the predicted peptides that have also been experimentally identified in our dataset, the probability score of the peptide identification is indicated. This allows one to select a potential glycopeptide based on its experimental identification or its predicted glycosylation site. Third, we determined the uniqueness of each predicted N-linked glycopeptide by searching for each sequence within the entire IPI protein database. Peptides present in multiple proteins are indicated by multiple database hits (FIG. 5, number of other proteins with the peptide). Uniqueness of a peptide sequence mapping to a particular protein within the human IPI database is taken to be a necessary condition for assigning a peptide to a protein identification and subsequent quantification (63).

### Example 3

### Quantitative Analysis of Proteins Secreted into the Extracellular Space of Prostate Cancer Tissues using SPEG and LC-MS/MS

Proteins present in the extracellular matrix contain proteins secreted from cells that are likely deposited into the blood. To identify proteins in the cell-free extracellular matrix of prostate cancer, samples (0.1 g) from patient-matched prostate cancer and adjacent control prostate tissues were processed by collagenase digestion into single cell suspensions, and the cell-free digestion media, containing secreted proteins in extracellular matrix, was analyzed. The samples were run on an SDS-PAGE gel. Silver staining showed minimal protein degradation, and a PSA Western blot showed a prominent reacting band at the expected molecular weight for PSA. To eliminate the analysis of abundant cytoplasmic proteins released from dead cells, the glycoproteins were isolated from the cell-free digestion media using SPEG. The isotopic labeled glycopeptides isolated from control and cancer tissues were then identified by LC-MS/MS. The MS/MS spectra were searched against the human database using SEQUEST. The identified proteins were quantified using the stable isotope quantification software, ASAPRatio (Li, X. J., Zhang, H., Ranish, J. A., and Aebersold, R. (2003) Anal Chem 75, 6648-6657). The results showed that all identified proteins were known to be secreted, thus validating the capture approach, and that the more abundant prostatic proteins of PAP and PSA were readily found. Other identified proteins included Igγ-2C, lumican, serum amyloid A-4, α-1-antitrypsin, plasma protease C1 inhibitor, complement C3, α-2-macroglobulin, haptoglobins, AMBP, α-1-antichymotrypsin, carboxypeptidase N chain, α-1-acid glycoprotein, TIMP1, complement C4, apolipoprotein B-100, kininogen, inter-α-trypsin inhibitor H4, complement C1q subcomponent, peptidoglycan recognition protein L, membrane copper amine oxidase, microfibril-associated glycoprotein 4, collagen α1, laminin γ1, acid ceramidiase, and zinc-α2-glycoprotein (ZAG). The protein with the best statistical score for differential expression in this experiment was TIMP1. The level of the identified glycopeptide from TIMP1 in cancer tissue was only 0.255 fold of that in control tissue.

Differential TIMP1 expression was next verified by Western blotting of cell-free media from cancer and normal prostate tissues using an anti-TIMP1 monoclonal antibody (clone 7-6C1, Chemicon). Equal amounts of protein (100 μg) from cell-free media of cancer and control tissues were separated on a 4-15% SDS-polyacrylamide gel (Bio-Rad), and transferred to Hybond-P membranes (Amersham Biosciences). The membranes were probed with anti-TIMP1. Anti-ZAG, (shown to be present in the same amount in cancer and control prostate samples by isotopic labeling and MS/MS analysis) (clone H-21, Santa Cruz Biotechnology) and anti-PSA (clone A67-B/E3, Santa Cruz Biotechnology) were also used to ensure equal loading of samples.

The amount of detectable TIMP1 in cancer tissue was several fold less than that in control tissue. A control blot using an antibody to ZAG showed that this protein was not differentially expressed between cancer and control tissue. Next, immunohistochemistry was carried out with this antibody. The staining result showed that TIMP1 was localized to luminal cells of benign glands (99-022H); tumor tissue had patchy or no staining of the cancer cells in the two cases with cancer (99-044A and 99-066C). The biological function of TIMP1 and other members of this class of inhibitors is to modulate the metalloproteinases (MMP) (Visse, R., and Nagase, H. (2003) Circ Res 92, 827-839). This finding correlates well with a published report on an increased ratio of MMP/TIMP1 in extracts of cancer vs. non-cancer prostate tissues (Jung, K., Lein, M., Ulbrich, N., Rudolph, B., Henke, W., Schnorr, D., and Loening, S. A. (1998) Prostate 34, 130-136). The imbalance is therefore due primarily to lowered TIMP-1 expression in cancer. As a consequence, the increased MMP activity may promote a number of processes that favor a cancerous state. These include degradation of extracellular matrix, tissue remodeling, release of factors beneficial to tumor establishment and growth, and neovascularization of the tumor tissue (McCawley, L. J., and Matrisian, L. M. (2000) Mol Med Today 6, 149-15). Not surprisingly, it has been shown that induced expression of TIMP1 in prostate cancer cells could suppress their invasive activity (Tachibana, K., Shimizu, T., Tonami, K., and Takeda, K. (2002) Biochem Biophys Res Commun 295, 489-494).

### Example 4

### Quantitative Analysis of Plasma Proteins with SPEG and LC-MS—Reducing the Complexity of Plasma-Derived Peptide Mixture and Increasing Sensitivity and Throughput

The selective isolation of the N-linked glycosylated peptides using SPEG results in a substantial improvement in the number of proteins detected and the concentration limit of detection since the complexity of the analyzed sample is significantly reduced. This is because the number of peptides per protein isolated by SPEG is significantly reduced. At constant detection sensitivity for the mass spectrometer used, the concentration limit for detection is directly dependent on the amount of sample applied to the capillary column of the LC-MS system. To estimate the extent of sample complexity reduction achieved by SPEG compared to the total unfractionated tryptic peptides, we analyzed plasma tryptic peptide samples generated with and without glycopeptide selection. The peptides were detected by a liquid chromatography electrospray ionization quadrupole-time-of-flight (LC-ESI-QTOF), in which the tryptic peptides from 50 nl of serum was applied. Fifty nl of plasma contains approximately 4 μg of protein, which represents the upper limit of loading capacity for the 75 μm i.d. capillary column used here. Indeed, the considerable streaking of highly abundant peptides in the horizontal axis indicated that the column capacity has already been reached or exceeded (Li, X. J., Pedrioli, P. G., Eng, J., Martin, D., Yi, E. C., Lee, H., and Aebersold, R. (2004) Anal Chem 76, 3856-3860), even at this low sample load. On the other hand, an equivalent display of a LC-MS run in which peptides recovered by SPEG from 5 μl of plasma sample were analyzed. From these data, it was immediately apparent that the pattern was much cleaner with better resolved peptides. Since 5 μl of plasma contains approximately 400 μg of protein, the glycopeptide capture strategy therefore allows for the analysis of 100 times more plasma in a single LC-MS analysis and thus the detection of lower abundance species compared to whole plasma analysis.

### Example 5

### Detection of Tumor-Specific P53 Sequences in Blood of Women with Ovarian Cancer

Investigators have been searching for molecular signatures from patients' blood to detect cancer early to improve patient's survival rate for ovarian cancer. Gene analyses of cancer have shown that alterations of several genes have been identified in a significant fraction of cancer patients, and tumor-specific DNA can be detected in cancer patients' blood samples for several cancer types (Nawroz, H., Koch, W., Anker, P., Stroun, M., and Sidransky, D. (1996) Nat Med 2, 1035-1037; Esteller, M., Sanchez-Cespedes, M., Rosell, R., Sidransky, D., Baylin, S. B., and Herman, J. G. (1999) Cancer Res 59, 67-70; Mulcahy, H. E., Lyautey, J., Lederrey, C., qi Chen, X., Anker, P., Alstead, E. M., Ballinger, A., Farthing, M. J., and Stroun, M. (1998) Clin Cancer Res 4, 271-275). p53 mutations are the most common single somatic alteration in ovarian cancer and occur in early as well as advanced staged disease (Okamoto, A., Sameshima, Y., Yokoyama, S., Terashima, Y., Sugimura, T., Terada, M., and Yokota, J. (1991) Cancer Res 51, 5171-5176; Kohler, M. F., Kerns, B. J., Humphrey, P. A., Marks, J. R., Bast, R. C., Jr., and Berchuck, A. (1993) Obstet Gynecol 81, 643-650). Mutations in p53 may be a sensitive indicator of the presence of circulating tumor DNA (Hibi, K., Robinson, C. R., Booker, S., Wu, L., Hamilton, S. R., Sidransky, D., and Jen, J. (1998) Cancer Res 58, 1405-1407; Silva, J. M., Dominguez, G., Garcia, J. M., Gonzalez, R., Villanueva, M. J., Navarro, F., Provencio, M., San Martin, S., Espana, P., and Bonilla, F. (1999) Cancer Res 59, 3251-3256). Using the tumor tissues and patient-matched blood samples collected by the University of Washington Gynecologic Oncology Tissue Bank, it has been found that somatic p53 mutations were detected in 69 of 137 tumors (50%). Forty-eight (70%) mutations were missense, occurring exclusively in exons 5-8. Twenty-one (30%) mutations were null mutations, consisting of 10 nonsense (14%), nine deletion (13%), and two splice site (3%) mutations. Twelve (17%) mutations occurred in exons 4 (N=7), 9 (N=2) or 10 (N=3).

Using ligase detection reaction for the 69 cases with somatic p53 mutations, the tumor-specific p53 sequences were detected in 21 plasma or serum samples (30%) from women with epithelial ovarian cancer. The results showed that the tumor DNA in plasma or serum was associated with patient prognosis and found that overall survival was significantly reduced in cases with tumor DNA in plasma (87). This indicated that free tumor DNA in plasma or serum was present in one-third of women with advanced ovarian cancer and was a strong independent predictor of decreased survival. The quantity of total DNA among women with ovarian cancer did not predict the presence of tumor-derived DNA sequences in plasma. Thus, simply quantifying DNA in plasma does not predict survival nor substitute for specific assays that identify tumor-derived sequences. Free tumor DNA in blood may represent a new biomarker in ovarian cancer. However, the poor sensitivity of circulating tumor DNA for identifying women with even advanced ovarian cancer points out the necessity of developing new protein-based biomarkers to create a blood-based test for ovarian cancer screening.

### Example 6

### High-Throughput Validation of Target Peptides in Plasma by Mass Spectrometry using Stable Isotope Labeled Synthetic Peptides

Once glycopeptides and proteins are identified from disease tissues, they will be detected and quantified in blood. Traditionally, antibodies recognizing these candidate proteins need to be used to detect the proteins. A mass spectrometry-based screening technology was developed that allows specific targeting of certain peptides/proteins with biological significance in a complex sample for identification and quantification. For each potential peptide identified from tissues, the identified formerly N-linked glycopeptide was chemically synthesized, labeled with at least one heavy isotope amino acid, and spiked in peptides isolated from plasma using SPEG. During MS analysis, this representative stable isotope labeled peptide standard distinguishes itself from the corresponding native peptide by a mass difference corresponding to the stable isotope label. Knowing the exact mass, sequence and quantity of the standard peptide, the peptide standard and its isotopic pair isolated from plasma can be located and selectively sequenced for identification, the quantification being achieved by the abundance ratio of spiked peptide to native peptide. Using specific mass matching to search the MS spectra, the spot (or spots) containing the peptide pairs was located. By examining the MS spectrum, the paired peaks (spiked and native) were determined. The identification of the peptides was further confirmed by MS/MS and SEQUEST database searching. The concentration of the native peptide was estimated from the abundance ratio of the peptide pair. Since this approach directly focuses on interesting peptides/proteins for identification and quantification, and the separation of peptide mixture for MALDI-TOF/TOF is done offline of a mass spectrometer, it technically increases the sample loading capacity, avoids some difficult issues associated with sample complexity, and thus significantly improves the throughput and sensitivity.

### Example 7

### Specific Enrichment of Target Peptides from Complex Samples to Increase Sensitivity using VICAT

VICAT reagents are a set of three related reagents, each with its own purpose (Bottari, P., Aebersold, R., Turecek, F., and Gelb, M. H. (2004) Bioconjug Chem 15, 380-388; Lu, Y., Bottari, P., Turecek, F., Aebersold, R., and Gelb, M. H. (2004) Anal Chem 76, 4104-4111). Each reagent contains an iodoacetamido group for selective attachment to the Cys sulfhydryl groups of peptides, and a biotinyl moiety for selective capture of tagged peptides using solid-phase streptavidin. One of the VICAT reagents, ¹⁴C-VICAT_{SH} (−28) is made “visible” by the fact that it contains a ¹⁴C-labeled methyl group. This facilitates our ability to track peptides or proteins tagged with these reagents using scintillation counting or autoradiography. Additionally, the ¹⁴C reagent is 28 mass units lighter than the non-radiolabeled VICAT_{SH} reagent, owing to the fact that the latter contains a diaminobutane linker rather than the ethylenediamine linker of the former. The third reagent VICAT_{SH} (+6) is chemically identical to VICAT_{SH} but is 6 mass units heavier due to the presence of 4 carbon-13 and 2 nitrogen-15 atoms in the diaminobutane linker. These mass differences are such that for a mixture of a single peptide labeled with all three, when run on an HPLC system, the VICAT_{SH}(+6) and VICAT_{SH} labeled peptides will co-migrate, but the ¹⁴C-VICAT_{SH}(−28) will resolve away from them by virtue of a shorter carbon chain. Finally, these reagents contain a photocleavable linker for release of tagged peptides from solid-phase streptavidin. After photocleavage, only a small fragment of the tag (including the isotope tag but not the radiolabel) is left attached to the cysteine SH group of the peptide (CH₂CONHCH₂CH₂CH₂CH₂NH₂ in the case of peptides tagged with VICAT_{SH}), and this group has 3 different masses so that the same peptide tagged with the 3 different VICAT_{SH} reagents are distinguishable in the mass spectrometer.

Preliminary data have proven this approach successful and superior to immunoblotting for absolute protein quantification, such as determining the absolute abundance of human group V phospholipase A2 (hGV) in human lung macrophages (Lu, Y., Bottari, P., Turecek, F., Aebersold, R., and Gelb, M. H. (2004) Anal Chem 76, 4104-4111). While immunoblot analyses were inconclusive, the application of VICAT allowed for isolation of hGV from whole cell lysate by following ¹⁴C-VICAT-labeled hGV peptides, and subsequent MS determination of an hGV concentration of 50 fmol per 100 μg of cell protein. By identification of potential cancer markers using large scale analysis of cancer tissues and plasma, the VICAT strategy can be used to enrich the target peptides from plasma and verify their association with cancer progression and with disease and control states, and for those of sufficient informational quality, provide invaluable absolute quantitative information (both concentration and range) to enable more rapid development of ELISA-based assays.

### Example 8

### Software Tools for Proteomic Data Analysis

Software tools for the analysis of the data generated by mass spectrometry have been generated. They include the following:

Peptide ProPhet: A tool that calculates accurate probabilities that a peptide has been correctly identified (Keller, A., Nesvizhskii, A. I., Kolker, E., and Aebersold, R. (2002) Anal Chem 74, 5383-5392).

Protein ProPhet: A tool that calculates accurate probabilities that a protein has been correctly identified based on the peptides matching to that protein (Nesvizhskii, A. I., Keller, A., Kolker, E., and Aebersold, R. (2003) Anal Chem 75, 4646-4658).

ASAPRatio: A tool for accurate quantification of peptides and proteins based on stable isotope ratios (Li, X. J., Zhang, H., Ranish, J. A., and Aebersold, R. (2003) Anal Chem 75, 6648-6657).

SpecArray: A tool to deconvolute the features detected by LC-MS into unique peptides and record each peak in three-dimensions (retention time, m/z, and intensity), to match peptides obtained from multiple analyses of different samples using LC-MS, and to quantify the matched peptides (Li, X. J., Yi, E. C., Kemp, C. J., Zhang, H., and Aebersold, R. (2005) Mol Cell Proteomics 4, 1328-1340).

PeptideAtlas and Plasma PeptideAtlas: A database mapping peptides derived from diverse proteomic experiments using tandem mass spectrometry (MS) data to eukaryotic genomes (PeptideAtlas) (Desiere, F., Deutsch, E. W., Nesvizhskii, A. I., Mallick, P., King, N. L., Eng, J. K., Aderem, A., Boyle, R., Brunner, E., Donohoe, S., Fausto, N., Hafen, E., Hood, L., Katze, M. G., Kennedy, K. A., Kregenow, F., Lee, H., Lin, B., Martin, D., Ranish, J. A., Rawlings, D. J., Samelson, L. E., Shiio, Y., Watts, J. D., Wollscheid, B., Wright, M. E., Yan, W., Yang, L., Yi, E. C., Zhang, H., and Aebersold, R. (2005) Genome Biol 6, R9), and a database mapping peptides identified from human plasma using tandem mass spectrometry data (Plasma PeptideAtlas) (Deutsch, E. W., Eng, J. K., Zhang, H., King, N. L., Nesvizhskii, A. I., Lin, B., Lee, H., Yi, E. C., Ossola, R., and Aebersold, R. (2005) Proteomics 5, 3497-3500).

### Example 9

### Determination of Peptides that are Ovary Tissue-Derived and Detectable from Blood using Glycopeptide Capture and Mass Spectrometry

Cancer cells differ from normal cells by the molecular and structural signatures that contribute to the cancer syndrome. The circulation of these molecular signatures may aid in monitoring cancer progression (as surrogate markers through their detection in body fluids). Secreted proteins and cell surface proteins from cancer cells are likely released into systemic circulation at low abundance and can be detected in blood. However, blood samples from individuals are expected to be more heterogeneous than cancer tissues since blood content can be affected by different physiological conditions such as age, sex, diet, and the time of the day at which the samples were collected. Due to these factors, identifying ovarian cancer biomarkers in plasma requires more targeted analyses of tissue-derived proteins in the background of other variations in the plasma proteome using a platform with high reproducibility and sensitivity.

General outline of the method: The reduced complexity and increased sensitivity (100-fold compared to unfractionated tryptic peptides of plasma proteins), throughput (96 sample preparations per week using the robotic system, and 30 sample analyses per week per mass spectrometer using LC-MS) and reproducibility (median CV <25% ((47)) using the robotic system for glycopeptide capture and automatic LC-MS analysis can be used to detect ovarian cancer-specific proteins in blood (47). Twenty pairs of ovarian cancer tissues and patient-matched blood samples collected prior to surgical therapy are analyzed. N-linked glycopeptides are analyzed from tissues and plasma samples, peptide patterns are generated by LC-MS or a list of identified peptides by LC-MS/MS, align and analyze the pattern for each patient, determine the common peptides from both tissue and plasma, and identify the peptide sequences. A list of peptides from each ovarian cancer tissue is generated with peptide characteristics such as mass, retention time, intensity, detectability in plasma, the stages at the surgery, and the clinical outcomes and other patient's information as related to the cancer case of each cancer tissue. A database will be established to store and query this information. This database provides the candidate proteins that can be further followed in a larger scale study using cancer tissues and blood samples collected longitudinally following primary surgical treatment. Since the same SPEG will be used in both tissue and plasma, the peptides and proteins can be compared in order to identify the maximum number of overlapping proteins present in the blood and the cancer tissue from the same patient.

Clinical samples: Twenty tissue-plasma pairs will be selected representing each stage of ovarian cancer (stage I to IV) and all of the common epithelial histologies (serous, mucinous, endometrioid, clear cell and undifferentiated). Tumors were surgically staged according to the International Federation of Obstetrics and Gynecology (FIGO) criteria (92). Blood was drawn pre-operatively and plasma frozen at −80C., All tissues will be from primary ovarian cancers without previous chemotherapy exposure.

Sample Preparation:

Purification of formerly N-linked glycosylated peptides from plasma using SPEG as described herein. Briefly, proteins from 200 μl of plasma samples in coupling buffer (100 mM NaAc and 150 mM NaCl, pH 5.5) are oxidized in 10 mM of sodium periodate at room temperature for 1 hour. After removal of sodium periodate by desalting column, the sample is conjugated to the hydrazide resin at room temperature for 10-24 hours. Non-glycoproteins are then removed by washing the resin 6 times with an equal volume of urea solution (8M urea/0.4M NH₄HCO₃, pH 8.3). After the last wash and removal of the urea solution, the resin is diluted with 3 bed volumes of water. Trypsin is added at a concentration of 1 μg of trypsin/200 μg of protein and digested at 37° C. overnight. The peptides are reduced by adding 8 mM TCEP (PIERCE, Rockford, Ill.) at room temperature for 30 min, and alkylated by adding 10 mM iodoacetamide at room temperature for 30 min. The trypsin-released peptides are removed by washing the resin three times with 1.5 M NaCl, 80% Acetonitrile, 100% methanol, and six times with 0.1 M NH₄HCO₃. N-linked glycopeptides are then released from the resin by addition of PNGase F (at a concentration of 1 μl of PNGase F/40 mg of protein) overnight. The released peptides are dried and resuspended in 0.4% acetic acid for MS analysis.

Cell surface and secreted proteins from tissues: The tissue is homogenized in 100 mM phosphate buffer (pH7.5) with 150 mM NaCl and 1% Triton X-100 on ice. The protein amounts will be measured using a BCA protein analysis kit (Pierce, Rockford, Ill.). Membrane proteins and secreted extracellular proteins will be specifically enriched from the total tissue lysate using SPEG described above to avoid the analysis of cytoplasmic proteins since surface proteins and secreted proteins are mostly glycosylated but cysoplasmic proteins are not. The same amounts of crude extracellular proteins will be used to isolate N-linked glycopeptides from each tissue sample.

Identify glycopeptides by LC-MS and LC-MS/MS from tissues and plasma samples and determine whether tissue-derived peptides can be detected in patient matched plasma sample

The isolated formerly N-linked glycopeptides (20 samples from tissues and 20 from patient-matched plasma) will be analyzed in three repeated analyses by LC-MS/MS using a linear ion trap mass spectrometer (LTQ, ThemoFinnigan, 120 runs) to achieve the highest sensitivity for sequencing of peptides present in tissues and plasma samples. MS/MS spectra obtained for these peptides will be used to identify the peptides by searching sequence databases using the SEQUEST software (48). The peptides identified only in tissue or plasma, and in both tissue and plasma can be determined by comparing the identified peptide lists and mass/retention time of peptide ions.

The glycopeptides isolated from plasma and tissues will also be analyzed by MALDI-TOF/TOF (ABI 4700 Proteomics Analyzer, Applied Biosystems) after front-end separation of peptides using reversed phase chromatography. The advantage of this platform is its high mass accuracy, resolution, throughput, sensitivity, and the ability to do targeted MS/MS analysis on peptides of interest. Since the separation is performed off-line, more peptide samples can be loaded onto the separation columns in order to increase the sensitivity. Multiple plates can also be spotted and analyzed by MALDI-TOF/TOF to increase the throughput. This platform will also be used in the direct follow up analysis of potential peptides during the cancer treatment using heavy isotope labeled synthetic peptide standards. Nano scale HPLC pumps will be used in both instruments for reproducible peptide elution patterns using reversed phase separation. The mass, retention time, and intensity of each identified peptide is determined using our recently developed SpecArray program (62). After pattern analysis, all the peptides from tissue and the common features in patient-matched plasma samples will be identified. The same MALDI plate will be reanalyzed and MS/MS spectra will be acquired at spots where the common peptides have been located from plasma sample for targeted MS/MS analysis using MALDI-TOF/TOF instrument.

Database for Identified Ovarian Cancer Tissue-Derived Peptides

A database will be established to allow exploration of each glycopeptide identified from ovarian cancer tissues. The database will display the identified peptide sequences and their proteins, their characteristics such as mass, retention time, intensity, their detectability in patient plasma, the stages of cancer in which the peptides are identified, and the cancer progression and clinical outcome for each cancer case. This database can be developed from our existing UniPep database, which displays all the potential and identified N-linked glycosylaltion sites for all proteins in protein database with additional fields for ovarian related information. The database will be linked to other protein and gene databases such as SwissProt, GeneCard, and EST database (dbEST) to allow users to explore the function of the protein, tissue specific expression, and any known relevant studies related to the disease.

### Example 10

### Mass Spectrometry-Independent Tests to Detect Ovarian Cancer Associated Proteins with Blood Samples and Improved Ability for Early Detection of Ovarian Cancer in the Relapsed Patient Population

In order to validate the candidate markers from ovarian tissues in large population of patients and determine the specificity and sensitivity of the candidate markers for ovarian cancer diagnosis prognosis, an assay for clinical use is developed. The results can be compared with the CA125 test in the same population of patients.

General outline of the method: Antibody-based detection methods are widely used in the clinical lab for CA125 test. A similar platform will be developed to detect the candidate cancer proteins using patients' blood samples longitudinally collected before and after therapy. Antibodies against the candidate proteins will be developed and used to test the protein in parallel with CA125 with blood samples. The capability to detect cancer at an earlier time of recurrence for better prognosis will be used to assess the value of the new test. If the protein of the candidate peptide can not be detected by an immunodetection method, the protein glycosylation changes (not total protein abundance) may be responsible for the detected difference. If this is the case, detection of the identified formerly N-linked peptides will be developed. We will assemble a test kit that includes the necessary reagents, plates with immobilized antibodies or peptides for clinical use.

ELISA test for proteins: Most serum tests are based on ELISA tests. The assay system utilizes two antibodies directed against different antigenic regions of the candidate protein. When the antibodies to the candidate protein are available, we will test whether the total protein amount is associated with cancer by developing an assay using ELISA. For example, a monoclonal antibody directed against a distinct antigenic determinant on the intact candidate protein is used for solid phase immobilization on the microtiter wells. A detection antibody conjugated to horseradish peroxidase (HRP) or fluorescence tag recognizes the candidate protein with different region of the same protein. The candidate protein reacts simultaneously with the two antibodies, resulting in the protein being sandwiched between the solid phase and detection antibody. The detection antibody can be visualized by color metric fluorescence analysis.

Test for peptides: In the case that 1) the formerly N-linked glycopeptide, but not the protein, is associated with ovarian cancer progression, or 2) two antibodies against the same proteins are not available or difficult to generate, we plan to develop tests for the cancer-specific candidate peptides identified and validated as described herein. In certain cases, the common sandwich ELISA test for proteins may not be applied to peptide antigens due to the small size of peptides to generate two antibodies against to the same short peptide sequence. In these cases, we plan to develop tests for the formerly N-linked glycopepetides as shown in FIG. 5.

The procedure has the following steps: 1) immobilize a certain amount of antibody against the specific peptide on the microtiter plate through immunoglobulin's carbohydrate groups leaving the antigen binding sites exposed to the surface, 2) dispense isolated peptides (from plasma of patients or controls), peptide antigen standards (with different concentrations) into appropriate wells and incubate, 3) add fluorescence labeled peptide antigen into each well and incubate, 4) wash the wells and read the plate with fluorescence plate reader. Optionally, the isolated peptides or peptide antigen standards can be labeled with different fluorescence tags before dispensing to the plate in step 2. Two different fluorescent colors can then be detected simultaneously for sensitive and accurate measurement (FIG. 5).

Test the candidate proteins/peptides with the plasma samples collected during the cancer therapy of ovarian cancer patients to determine their ability to detect cancer recurrence early: Once the test is developed, the complete reagents as a testing kit are made that can be used in clinical labs. The tests will be applied to plasma samples from retrospectively collected plasma samples, and the prospective plasma samples collected during the project. The sensitivity of detecting recurrent cancer at earlier timepoints compared to CA125 and the ability of the new marker to complement CA125 will be used to assess the value of the new tests. In samples obtained at diagnosis, the candidate markers can also be tested for prognostic value taking into account other prognostic factors (stage, age, adequacy of surgical cytoreduction).

### Example 11

### Direct Follow-Up Analysis of Overlapping Peptides in Blood to Determine Response to Primary Cancer Therapy and Association with Cancer Recurrence using Synthetic Heavy Isotope Labeled Peptides

A list of formerly N-linked glycopeptides detected in both ovarian cancer tissues and their patient-matched plasma samples from different clinical stages and outcome of cancer progression will be identified as described herein. These peptides have the potential to be blood biomarkers to detect ovarian cancer. They can be derived from normal ovary cells, early curable stage and chemo-sensitive ovarian tumor cells, or late stage and chemo-resistant ovarian cancer cells. They will be further investigated in blood samples from normal and ovarian cancer patients along the following lines: 1) the identified peptides and proteins are verified using different platforms than the original LC-MS-based discovery approach. 2) The relationship of each peptide in blood with ovarian cancer progression after primary surgical therapy is established. 3) The specificity and sensitivity of candidate markers is determined by screening suitable populations of human plasma samples from patients with ovarian cancer and appropriate controls. These require a high throughput analysis of a large number of proteins identified from tissue and blood. Immuno assays using specific antibodies are commonly used in validation studies of proteins. However, in certain embodiments, it may be desirable to use synthetic peptides with heavy isotope labeling for the following reasons 1) the abundance of glycopeptides identified from tissues and blood samples reflects the abundance of the a glycoprotein and the occupancy of a specific glycosylation site of the peptide, therefore total protein analysis using antibody against the protein may not detect the relevance of the specific glycopeptides identified; 2) Antibodies may not available to all proteins; 3) The synthetic peptide maintains the same characteristics of the native peptide; the chromatographic retention time and the MS/MS spectrum of the synthetic peptide can be used to identify a specific peptide while the heavy isotope labeling allows the quantification of the peptide using mass spectrometry.

General Outline of the Method:

The peptides identified from ovarian cancer tissues are tested to determine if the the peptides are biomarkers in blood. Longitudinally collected blood samples from 50 patients are analyzed and compared to the performance of the potential proteins with serum CA125, which is measured from the same patients

We will quantify and identify every selected glycopeptide identified in both ovarian cancer tissues and patient-matched plasma using plasma samples before and after primary surgical therapy. The heavy isotope-labeled version of the selected peptides will be synthesized and spiked into glycopeptides isolated from plasma samples. The peptides then can be separated and analyzed by LC-MS and LC-MS/MS as shown previously (61)

Prospective collection of clinical samples: We will enroll 50 cases with advanced ovarian cancer (stage III or IV). Approximately 60% of women with advanced ovarian cancers will be optimally debulked (residual tumor <1 cm in greatest diameter) at the time of initial surgery. Thus, we expect to enroll 30 women with optimally debulked disease and 20 women with suboptimally debulked (residual tumor >1 cm in diameter) disease. Blood will be collected pre-operatively, three months after surgery and then every six months after surgery until clinical diagnosis of recurrence. Patient clinical follow-up will be obtained until death. We will send subjects blood collection and shipping kits prior to each blood draw. The blood samples of greatest utility for testing potential diagnostic markers are those obtained during clinical remission at defined intervals prior to recurrence. The most useful samples are from women who have a complete response to chemotherapy and then to have a recurrence. Rate of chemotherapy response (CR) and recurrence varies based on the adequacy of surgical cytoreduction from optimal and suboptimal disease (94, 95). Of those 50 enrolled cases, we would expect 39 women to have complete chemotherapy response (13 from suboptimal disease and 26 from optimal disease) and 27 of these women with recur within 36 months of the study interval (FIG. 18). If 10% of women drop off the study we should have approximately 25 women who recur during the study interval and approximately 200 blood samples collected from these women. Blood from 100 age-matched normal individuals without history of previous cancer will also be collected as normal controls.

Synthesis and Labeling of Peptide Standards:

Candidate peptides to be synthesized and validated are selected using the following criteria: 1) the peptide presents in most tissue and plasma pairs at a specific stage; 2) the peptides are ovarian cancer cell derived rather than from classic plasma proteins from blood circulation; 3) peptides from proteins that have shown to be ovary-specific from literature or database will be given priority. During the chemical synthesis, the peptide is labeled with heavy ¹³C-and ¹⁵N-labeled D in the position where the deglycosylated D is generated from formerly N-linked glycosylated N. Since all the formerly N-linked glycopeptides contain D in the previous N—X—T/S motif, all the heavy isotope-labeled synthetic peptides will obtain a mass differential of 5 mass units from the normal peptides in plasma.

Quantitative analysis of the ovarian cancer tissue-derived peptides in plasma samples using heavy isotope labeled peptides and mass spectrometry: The synthetic peptides will be used as standards to quantify the candidate peptides from plasma samples (96). A mixture of 100 synthetic peptides with 10 fmole of each peptide is spiked into the peptides isolated from plasma samples. The peptides are spotted on MALDI plate from reversed phase separation. In this case, the mass spectrometer (MALDI-TOF/TOF) will be used to acquire a MS scan of the peptides. The known peptide mass of spiked standard heavy peptides and their light isotopic pairs isolated from plasma samples will be included in the inclusion list to acquire MS/MS spectra. The specific peptides are identified using SEQUEST search (96). Since multiple isotopically labeled synthetic peptides with known sequences, amount of peptide, retention time, and MS/MS spectrum can be used in each LC-MS and LC-MS/MS analysis to identify and quantify the peptides isolated from plasma, this method increases the throughput by allowing multiplexing.

A representative peptide corresponding to plasma membrane-associated protein was spiked into glycopeptides isolated from ovarian tissue where this peptide was originally identified and analyzed the sample by LC-MS and LC-MS/MS to validate the identification and quantification of the peptide. The synthetic peptide maintained the same characteristics as the normal peptide including the same chromatographic retention time and MS/MS spectra. The fragmentation of the synthetic peptide matched with the MS/MS spectrum derived from a normal peptide isolated from ovarian cancer tissue (97), save for the mass difference required for accurate quantification. Thus such heavy isotope labeled standard peptides could be used to verify and quantify many plasma proteins via MS using a high-throughput platform as recently demonstrated (61) on account of 1) the co-elution of the heavy isotope synthetic peptide and its light native form, 2) the similarity of the MS/MS spectra, and 3) and abundance ratio of light and heavy peptides. For this purpose, we have synthesized heavy isotope-labeled peptides that represent over 300 glycosylation sites, and they were listed with the corresponding proteins in UniPep database (63)). This is a gel-free and antibody-free approach for high-throughput peptide detection and quantification of previously identified peptides from tissues in plasma using synthetic peptides and mass spectrometry.

Data Analysis

We will analyze the relative abundance of each potential peptide identified in both ovarian tissue and plasma and quantitatively determine the response of each peptide in terms of clinical outcome during the disease development after primary surgical therapy and during chemotherapy. It is expected that ovarian tissue-derived peptides can have different responses during cancer progression: 1) Ubiquitously expressed proteins-the relative abundance of their peptides stays relatively unchanged after surgery (3 month after surgery and treatment vs 0 month before surgery) and no significant differences in case (0 month) vs control groups; 2) Ovary-specific but not cancer-associated -the relative abundance of their peptides decreases after surgical removal of ovary (3 month after surgery and treatment vs 0 month), but there is no significant difference in case (0 month) vs control groups; 3) Ovary-specific proteins associated with treatable disease-the relative abundance of their peptides decreases after surgical removal of ovarian cancer and stay low during chemotherapy; The level of proteins is higher in case vs control. These proteins may also be detected in patients with early stage cancer and the group of patients without cancer recurrence; 4) Ovary-specific proteins associated with resistant disease: the relative abundance of the peptide decreases after surgical removal of ovarian cancer and come back during chemotherapy after initial decrease due to the surgery. The level of the peptides is higher in case vs control.

### Example 12

### Improved Detection Limit of Low Abundance Tissue-Derived Peptides that are Undetectable in Blood via Direct Mass Spectrometry Analysis

The glycopeptides identified from ovarian cancer tissue but not detected in plasma using direct MS analysis may represent low abundant proteins released in small amounts from cancer tissues (see Table 1). Detecting these low abundance proteins in blood may increase the capability of detecting a cancer marker in an early stage of cancer, which is critical for cancer screening. To detect these ovarian cancer tissue-derived peptides that are not detectable in plasma by direct LC-MS analysis, a more sensitive method or targeted enrichment is used to increase the sensitivity of detecting these peptides in plasma.

General outline of the method: Immunoassays combined with fluorescence detection can be a sensitive method to detect proteins, if the antibodies are available. In this case, an enzyme-linked immunosorbent assay (ELISA) can be developed. In the case of peptides identified from cancer tissue need to be detected in blood, the specific peptide can be further enriched from peptide mixture isolated from plasma using the physico-chemical properties of the peptide or affinity reagents developed for the peptide.

The enzyme-linked immunosorbent assay (ELISA) system represents a reliable and sensitive method for detection and monitoring of a protein in blood and can be developed into a standard clinical laboratory assay. It requires pair-wise, well-characterized, high-affinity antibodies directed against a distinct antigenic determinant on the protein or peptide.

Immunoaffinity capture of glycopeptides can be used to increase the sensitivity and specificity of detecting candidate peptides in plasma samples, if further simplification beyond the SPEG method is required for detecting candidate peptides in plasma samples. This method has been shown to provide enrichment of specific peptides (97, 98, 99). Antibodies are generated against formerly N-linked glycopeptides from each candidate peptide. The antibody will be used to capture specific (glyco)peptides from a peptide mixture isolated from plasma using SPEG as well as the heavy isotopic labeled synthetic peptide standard spiked in the peptide mixture. The detection and quantification process can be described as the following steps: 1) The identified formerly N-linked glycopeptides are synthesized; 2) The synthetic peptides are used to produce antibodies; 3) The antibodies are immobilized on solid support; 4) Peptides from plasma are purified using SPEG; 5) Known amounts of heavy isotope tag-labeled peptides are spiked to the light isotope tag-labeled peptides isolated from plasma; 6) The immobilized antibodies for each glycopeptide are incubated with a binding solution containing peptides from step 5, and the resin is washed to remove peptides with nonspecific binding; 7) The affinity-captured peptides are detected by mass spectrometry; 8) The presence of light isotopic peptides and the ratio of biological light and in vitro-added heavy isotope tagged peptides are determined. Alternatively, the standard peptide can be labeled with fluorescence and spiked into the glycopeptides isolated from plasma. After affinity isolation, the peptide present in plasma can be quantified using a fluorometer (see e.g., FIG. 5).

Many protein biomarkers in the early stage of cancer development are present at exceedingly low concentrations. The detection of these proteins is generally difficult because of the “top down” operation mode of most current proteomics techniques. The antibody to a potential peptide marker can specifically capture the peptide of interest and remove other peptides from the analysis. This increases the sensitivity of the analysis. In addition, because the mass of the peptide from each enrichment is known, the mass spectrometer can focus on only scanning for the known mass, and therefore increase the sensitivity 10- to 100-fold. The detection of a known peptide mass from each affinity capture eliminates the detection of other peptides that bind to the antibody non-specifically, increasing the specificity and accuracy of quantification. The introduction of the heavy isotope-tagged peptides in the analysis also increases the accuracy of quantification, and serves as a positive control for the detection of the light isotopic form of a peptide in the biological sample. This differentiates real biological variation from experimental variation, and increases the confidence of the results.

Enrichment and verification of candidate markers using VICAT. The complexity of peptides isolated by SPEG can be further simplified by using VICAT reagents as described in preliminary results. VICAT will be employed in the following way. The amino groups of (glyco)peptides isolated by SPEG will be thioacetylated to 2-sulfhydryl-acetamido group, which then can be tagged by VICAT reagents (88). This step is necessary, since most formerly N-linked glycopeptides isolated by SPEG do not contain Cys, which are required for VICAT tagging. After thioacetylation of amino groups of synthetic peptides and of peptides isolated from plasma samples, the peptides isolated from plasma samples will be tagged with the VICAT_{SH} reagent. A known amount of a synthetic peptide standard, with the sequence of the target candidate peptide, will be tagged with VICAT_{SH}(+6). The same synthetic peptide will also be tagged with ¹⁴C-VICAT_{SH} (−28). A sufficient quantity of the latter standard, referred to as the chromatographic marker, is added to ensure that it can be tracked during chromatographic or electrophoretic separation. After peptide tagging with VICAT reagents, peptides isolated from plasma samples, the standard peptide, and the chromatographic marker are mixed and separated by isoelectric focusing (IEF) or other separation methods. The peptide fraction containing the target peptides visualized via the radioactively labeled chromatographic marker will be collected and peptides will be analyzed by mass spectrometry. Only the fraction that contains the targeted peptide is collected and further analyzed, it will significantly simplify the peptide complexity and make it possible to detect lower abundance specifically tagged peptides in highly complex plasma protein mixtures.

### Example 13

### Detection of Low Abundant Peptides in Blood and Early Detection of Disease by their Association with Primary Cancer Therapy and Cancer Recurrence

The low abundance tissue-derived peptides present in plasma may come from proteins released in small amount from cancer tissues. The increased sensitivity using the method developed herein will allow us to detect these peptides and determine whether they are associated with primary cancer therapy and can be used as markers to diagnose cancer at early stage or as indicator of progressive disease.

Once a specific enrichment method is developed for each peptide and the peptide can be detected in plasma using the improved method, we will determine the association of the these peptides with therapy and disease recurrence. These can be achieved using the same glycopeptides isolated from plasma samples longitudinally collected from cancer patients before and after primary cancer surgery. The only difference in this case is that a specific enrichment method for the target peptide or protein will be used to analyze the samples from plasma. Once a candidate marker is identified, a specific assay to detect the marker in plasma is developed as described elsewhere herein.

### Example 14

### Improvements to the Glycocapture Method: Glycoprotein Capture Versus Glycopeptide Capture

This Example describes the comparison of the glycocapture method essentially as described in US Patent Application Publication 20040023306 and a glycopeptide capture method. The results indicate that the glycopeptide capture method provides significant improvements in overall yield as well as specificity of capture.

Solid phase capture of glycosylated peptides can be achieved either from intact glycoproteins or glycopeptides. It is thought that glycopeptide capture is better, since there is no steric hinderance preventing binding of multiple glycosylation sites (as with intact glycoproteins). Another advantage to glycopeptide capture is that hydrophobic membrane proteins generally are not very soluble during glycoprotein capture. However, glycopeptides derived from the same membrane proteins will more likely exhibit favorable solubility thereby enabling enhanced capture.

The comparison between glycoprotein capture and glycopeptide capture was carried out as follows:

Reagents:

10× coupling buffer: 50 mM EDTA, 400 mM Tris pH 8.0.

Sixty uL multiple affinity removal system (MARS) depleted serum (600 ugs) was diluted with 20 uL 10× coupling buffer, 6 uL fetuin and 110 uL water. Four uL 500 mM TCEP (10 mM final concentration) was added and the mixture incubated at room temperature (RT) for 30 minutes. 96 mg urea was added and the mixture incubated for 30 minutes at RT. 4 uL of 250 mM iodoacetamide was added and the mixture incubated for an additional 30 min at RT. 0.5 uL 1M DTT was added and the mixture incubated for 20 min at RT. The urea in the sample was diluted by adding 1 mL 40 mM Tris pH 8.0. 10 ug of sequencing grade trypsin was added and the sample incubated with constant mixing overnight at 37° C. The sample was then acidified by adding 25 uL 10% TFA. The pH was checked using paper strips.

The sample was then cleaned up by reverse phase as follows: C-18 spin columns (Macrospin column from Harvard Apparatus, Holliston, Mass.) were hydrated with 500 uL 60% ACN 0.1% TFA. Columns are then washed three times with 500 uL 2% ACN 0.1% TFA. The sample was loaded and spun. The sample was passaged twice to collect all the protein. The columns were then washed three times with 200 uL 0.1% TFA. The proteins were eluted from the column with 3×75 uL of 60% ACN, 0.1% TFA. The eluate was collected and dried using a speedvac. The dried peptides were resuspended in 160 uL 1× coupling buffer.

Forty uL 10 mg/mL sodium periodate was added for 30 minutes at RT. The oxidized sample was added to 500 uL of pre-equilibrated hydrazide beads (50% slurry in coupling buffer) and incubated at RT overnight with constant mixing. The unbound fractions were collected and stored. The bound proteins (resin) were washed twice with 1 mL of water, 1.5 M NaCl, methanol, 80% ACN, 100 mM ammonium bicarbonate (AMBIC).

After the final wash, the beads were resuspended in 300 uL of 100 mM AMBIC containing 1 uL PNGaseF ((peptide: N-glycosidase F [EC 3.5.15.2, N-linked-glycopeptide-(N-acetyl-beta-D-glucosaminyl)-L-asparagine amidohydrolase]) is an amidase which cleaves between the innermost GlcNAc and asparagine residues of high mannose, hybrid and complex oligosaccharides from N-linked glycoproteins). The beads are then incubated overnight at 37° C. with constant agitation.

Following the overnight incubation, the supernatant fraction is collected and transfered to fresh tubes. The resin was washed twice with 100 uL 80% CAN. The washes were collected each time and transferred to eluted fraction. The sample was then dried down in a speed-vac.

The samples were resuspended in water and desalted using a reverse phase column prior to cation exchange and MS analyses.

The comparison experiment was designed as follows: The commonly used glycoprotein control, Fetuin, was spiked into two background protein mixtures (CL1 cell lysate and serum) such that fetuin was 5% by weight. Each sample (CL1 and serum) was split into two fractions where one was subjected to the usual glycoprotein capture as described in US Patent Application Publication No. 20040023306 and the other was subjected to the glycopeptide capture method described above. Ninety-six pmol of a stable isotope labelled fetuin peptide (LCPDCPLLAPLDDSR (SEQ ID NO:14,918), with carbamidomethylated cysteine and ¹³C and ¹⁵N labelling of the C-terminal R) containing the N-linked site (but with the N converted to D) was spiked into the samples that contained 1092 pmol of fetuin. The samples containing the internal standard were subjected to solid phase extraction prior to Maldi-Tof analysis. Comparing the ratios of ion abundances of the internal standard versus fetuin peptide for glycopeptide and glycoprotein capture showed that the glycopeptide capture had a 20-30 fold higher yield (same results for serum or CL1 background). Similar results were obtained when analyzed by LC-Maldi.

The serum glycoprotein and glycopeptide captures were also analyzed by LCMSMS using the 4800 Maldi TofTof, and the resulting MSMS spectra obtained by data dependent analysis. The MSMS spectra were identified using Mascot.

The results showed that there are a large number of non-glycosylated peptides in the serum glycoprotein capture, but very few in the glycopeptide capture (ie, the selectivity of the glycopeptdie capture is higher). Also, the probability scores in the glycopeptide capture are much higher than for the same peptides in the glycoprotein capture, which is most likely due to higher intensity precursor ions resulting from higher capture yields. It should be noted that although glycopeptides containing N-terminal Ser or Thr are present in the glycoprotein capture list, they are absent from the glycopeptide list. This is most likely due to oxidation of the vicinal amino and hydroxyl groups. This reaction could be eliminated by first derivatizing amino groups.

In summary, these experiments indicate that glycopeptide capture is superior to glycoprotein capture with respect to yield and specificity of capture. Indeed, a direct comparison of the two procedures indicates a 20-30 fold higher yield than the glycoprotein method. The absolute yield for each of the procedures remains to be determined.

With respect to the specificity of glycopeptide identification, the peptides derived from the top twenty identified proteins from each procedure from a serum sample were examined. Glycoprotein capture resulted in the identification of 40 peptides with high confidence, of these 13 contained the N—X—S glycosylation motif, a specificity of 33%. Glycopeptide capture identified 50 peptides containing a consensus glycosylation site from 45 identified peptides (90% specificity). A more pronounced difference was observed for CL1 whole cell lysates, where none of the peptides from a glycoprotein capture experiment contained N-linked consensus sites, whereas nearly the opposite was true for glycopeptide capture (only 2 out of 27 were not glycopeptides). Both of these findings (higher yield and specificity) are a significant advancement to the technology of glycocapture. As noted above, glycopeptides containing N-terminal Ser or Thr cannot be identified by the glycopeptide capture approach, since periodate converts the Ser or Thr to an aldehyde that either is dispersed via reactions with side chains from other peptides, or is permanently attached to the hydrazide bead. As such, no N-terminal Ser nor Thr containing peptides were identified by this method. Furthermore, data exists showing the presence of the oxidized Ser on specific peptides (both MS and MSMS).

### REFERENCES

1. R. Etzioni et al., Nat Rev Cancer 3, 243 (April 2003).

2. E. E. Schadt et al., Nat Genet 37, 710 (July 2005).

3. H. Dai et al., Cancer Res 65, 4059 (May 15, 2005).

4. N. L. Anderson, N. G. Anderson, Mol Cell Proteomics 1, 845 (November 2002).

5. R. S. Tirumalai et al., Mol Cell Proteomics 2, 1096 (October 2003).

6. D. Nedelkov, U. A. Kiernan, E. E. Niederkofler, K. A. Tubbs, R. W. Nelson, Proc Natl Acad Sci USA 102, 10852 (Aug. 2, 2005).

7. E. P. Diamandis, Mol Cell Proteomics 3, 367 (April 2004).

8. H. Zhang, X. J. Li, D. B. Martin, R. Aebersold, Nat Biotechnol 21, 660 (June 2003).

9. C. D. Hough et al., Cancer Res 60, 6281 (Nov. 15, 2000).

10. C. D. Hough, K. R. Cho, A. B. Zonderman, D. R. Schwartz, P. J. Morin, Cancer Res 61, 3869 (May 15, 2001).

11. J. Eng, A. L. McCormack, J. R. Yates, 3rd, J. Am. Soc. Mass Spectrom. 5, 976 (1994).

12. X. J. Li, E. C. Yi, C. J. Kemp, H. Zhang, R. Aebersold, Mol Cell Proteomics 4, 1328 (September 2005).

13. A. Krogh, B. Larsson, G. von Heijne, E. L. Sonnhammer, J Mol Biol 305, 567 (Jan. 19, 2001).

14. L. D. True, A. Y. Liu, Am J Clin Pathol 120, 13 (July 2003).

15. W. Weichert, T. Knosel, J. Bellach, M. Dietel, G. Kristiansen, J Clin Pathol 57, 1160 (November 2004).

16. I. Kholova, A. Ryska, M. Ludvikova, L. Pecen, J. Cap, Cas Lek Cesk 142, 167 (March 2003).

17. G. Kristiansen et al., Prostate 54, 34 (Jan. 1, 2003).

18. G. P. Murphy et al., Cancer 78, 809 (Aug. 15, 1996).

19. G. Murphy et al., Anticancer Res 15, 1473 (July-August 1995).

20. K. Leitzel et al., J Clin Oncol 10, 1436 (September 1992).

21. A. Marchetti et al., Cancer Res 62, 2535 (May 1, 2002).

22. A. Y. Liu, H. Zhang, C. M. Sorensen, D. L. Diamond, J Urol 173, 73 (January 2005).

23. H. Zhang et al., Mol Cell Proteomics 4, 144 (February 2005).

24. Xu, Y., Shen, Z., Wiper, D. W., Wu, M., Morton, R. E., Elson, P., Kennedy, A. W., Belinson, J., Markman, M., and Casey, G. (1998) Jama 280, 719-723

25. Anderson, N. L., and Anderson, N. G. (2002) Mol Cell Proteomics 1, 845-867

26. Jemal, A., Murray, T., Ward, E., Samuels, A., Tiwari, R. C., Ghafoor, A., Feuer, E. J., and Thun, M. J. (2005) CA Cancer J Clin 55, 10-30

27. Kennedy, A. W., and Hart, W. R. (1996) Cancer 78, 278-286

28. Jones, M. B., Krutzsch, H., Shu, H., Zhao, Y., Liotta, L. A., Kohn, E. C., and Petricoin, E. F., 3rd. (2002) Proteomics 2, 76-84

29. Niloff, J. M., Klug, T. L., Schaetzl, E., Zurawski, V. R., Jr., Knapp, R. C., and Bast, R. C., Jr. (1984) Am J Obstet Gynecol 148, 1057-1058

30. Meyer, T., and Rustin, G. J. (2000) Br J Cancer 82, 1535-1538

31. Welsh, J. B., Zarrinkar, P. P., Sapinoso, L. M., Kern, S. G., Behling, C. A., Monk, B. J., Lockhart, D. J., Burger, R. A., and Hampton, G. M. (2001) Proc Natl Acad Sci USA 98, 1176-1181

32. Schadt, E. E., Lamb, J., Yang, X., Zhu, J., Edwards, S., Guhathakurta, D., Sieberts, S. K., Monks, S., Reitman, M., Zhang, C., Lum, P. Y., Leonardson, A., Thieringer, R., Metzger, J. M., Yang, L., Castle, J., Zhu, H., Kash, S. F., Drake, T. A., Sachs, A., and Lusis, A. J. (2005) Nat Genet 37, 710-717

33. Dai, H., van't Veer, L., Lamb, J., He, Y. D., Mao, M., Fine, B. M., Bernards, R., van de Vijver, M., Deutsch, P., Sachs, A., Stoughton, R., and Friend, S. (2005) Cancer Res 65, 4059-4066

34. Warrenfeltz, S., Pavlik, S., Datta, S., Kraemer, E. T., Benigno, B., and McDonald, J. F. (2004) Mol Cancer 3, 27

35. Hough, C. D., Sherman-Baust, C. A., Pizer, E. S., Montz, F. J., Im, D. D., Rosenshein, N. B., Cho, K. R., Riggins, G. J., and Morin, P. J. (2000) Cancer Res 60, 6281-6287

36. Aebersold, R., and Mann, M. (2003) Nature 422, 198-207

37. Wulfkuhle, J. D., Liotta, L. A., and Petricoin, E. F. (2003) Nat Rev Cancer 3, 267-275

38. Diamandis, E. P. (2004) Mol Cell Proteomics 3, 367-378

39. Petricoin, E. F., Ardekani, A. M., Hitt, B. A., Levine, P. J., Fusaro, V. A., Steinberg, S. M., Mills, G. B., Simone, C., Fishman, D. A., Kohn, E. C., and Liotta, L. A. (2002) Lancet 359, 572-577

40. Adkins, J. N., Varnum, S. M., Auberry, K. J., Moore, R. J., Angell, N. H., Smith, R. D., Springer, D. L., and Pounds, J. G. (2002) Mol Cell Proteomics 1, 947-955

41. Tirumalai, R. S., Chan, K. C., Prieto, D. A., Issaq, H. J., Conrads, T. P., and Veenstra, T. D. (2003) Mol Cell Proteomics 2, 1096-1103

42. Shen, Y., Jacobs, J. M., Camp, D. G., 2nd, Fang, R., Moore, R. J., Smith, R. D., Xiao, W., Davis, R. W., and Tompkins, R. G. (2004) Anal Chem 76, 1134-1144

43. Wang, H., and Hanash, S. (2003) J Chromatogr B Analyt Technol Biomed Life Sci 787, 11-18

44. Shin, B. K., Wang, H., and Hanash, S. (2002) J Mammary Gland Biol Neoplasia 7, 407-413

45. Villanueva, J., Philip, J., Entenberg, D., Chaparro, C. A., Tanwar, M. K., Holland, E. C., and Tempst, P. (2004) Anal Chem 76, 1560-1570

46. Zhang, H., Li, X. J., Martin, D. B., and Aebersold, R. (2003) Nat Biotechnol 21, 660-666

47. Zhang, H., Yi, E. C., Li, X. J., Mallick, P., Kelly-Spratt, K. S., Masselon, C. D., Camp, D. G., 2nd, Smith, R. D., Kemp, C. J., and Aebersold, R. (2005) Mol Cell Proteomics 4, 144-155

48. Eng, J., McCormack, A. L., and Yates, J. R., 3rd. (1994) J. Am. Soc. Mass Spectrom. 5, 976-989

49. Han, D. K., Eng, J., Zhou, H., and Aebersold, R. (2001) Nat Biotechnol 19, 946-951

50. Keller, A., Nesvizhskii, A. I., Kolker, E., and Aebersold, R. (2002) Anal Chem 74, 5383-5392

51. Li, X. J., Zhang, H., Ranish, J. A., and Aebersold, R. (2003) Anal Chem 75, 6648-6657

52. Nesvizhskii, A. I., Keller, A., Kolker, E., and Aebersold, R. (2003) Anal Chem 75, 4646-4658

53. Zhang, H., Yan, W., and Aebersold, R. (2004) Curr Opin Chem Biol 8, 66-75

54. Casey, R. C., Oegema, T. R., Jr., Skubitz, K. M., Pambuccian, S. E., Grindle, S. M., and Skubitz, A. P. (2003) Clin Exp Metastasis 20, 143-152

55. Catterall, J. B., Jones, L. M., and Turner, G. A. (1999) Clin Exp Metastasis 17, 583-591

56. Walker, B. K., Lei, H., and Krag, S. S. (1998) Biochem Biophys Res Commun 250, 264-270

57. Couldrey, C., and Green, J. E. (2000) Breast Cancer Res 2, 321-323

58. Pieper, R., Su, Q., Gatlin, C. L., Huang, S. T., Anderson, N. L., and Steiner, S. (2003) Proteomics 3, 422-432

59. Putnam, F. (1975) The plasma proteins: Structure, Function, and Genetic Control, 2nd ed., Academic Press, New York, N.Y.

60. Nedelkov, D., Kiernan, U. A., Niederkofler, E. E., Tubbs, K. A., and Nelson, R. W. (2005) Proc Natl Acad Sci USA 102, 10852-10857

61. Pan, S., Zhang, H., Rush, J., Eng, J., Zhang, N., Patterson, D., Comb, M. J., and Aebersold, R. (2005) Mol Cell Proteomics 4, 182-190

62. Li, X. J., Yi, E. C., Kemp, C. J., Zhang, H., and Aebersold, R. (2005) Mol Cell Proteomics 4, 1328-1340

63. Zhang, H., Loriaux, P., Eng, J., Keller, A., Moss, P., Bonneau, R., Yi, E. C., Lee, H., Cooke, K., and Aebersold, R. (2005) submitted

64. Zhang, H., Liu, A. Y., Loriaux, P., Wollscheid, B., Zhou, Y., Watts, J., and Aebersold, R. (2005) submitted

65. Liu, A. Y., Zhang, H., Sorensen, C. M., and Diamond, D. L. (2005) J Urol 173, 73-78

66. Roth, J. (2002) Chem Rev 102, 285-303

67. Petrescu, A. J., Milac, A. L., Petrescu, S. M., Dwek, R. A., and Wormald, M. R. (2004) Glycobiology 14, 103-114

68. Hough, C. D., Cho, K. R., Zonderman, A. B., Schwartz, D. R., and Morin, P. J. (2001) Cancer Res 61, 3869-3876

69. Krogh, A., Larsson, B., von Heijne, G., and Sonnhammer, E. L. (2001) J Mol Biol 305, 567-580

70. Nielsen, H., Engelbrecht, J., Brunak, S., and von Heijne, G. (1997) Protein Eng 10, 1-6

71. True, L. D., and Liu, A. Y. (2003) Am J Clin Pathol 120, 13-15

72. Weichert, W., Knosel, T., Bellach, J., Dietel, M., and Kristiansen, G. (2004) J Clin Pathol 57, 1160-1164

73. Kholova, I., Ryska, A., Ludvikova, M., Pecen, L., and Cap, J. (2003) Cas Lek Cesk 142, 167-171

74. Kristiansen, G., Pilarsky, C., Wissmann, C., Stephan, C., Weissbach, L., Loy, V., Loening, S., Dietel, M., and Rosenthal, A. (2003) Prostate 54, 34-43

75. Visse, R., and Nagase, H. (2003) Circ Res 92, 827-839

76. Jung, K., Lein, M., Ulbrich, N., Rudolph, B., Henke, W., Schnorr, D., and Loening, S. A. (1998) Prostate 34, 130-136

77. McCawley, L. J., and Matrisian, L. M. (2000) Mol Med Today 6, 149-156

78. Tachibana, K., Shimizu, T., Tonami, K., and Takeda, K. (2002) Biochem Biophys Res Commun 295, 489-494

79. Li, X. J., Pedrioli, P. G., Eng, J., Martin, D., Yi, E. C., Lee, H., and Aebersold, R. (2004) Anal Chem 76, 3856-3860

80. Nawroz, H., Koch, W., Anker, P., Stroun, M., and Sidransky, D. (1996) Nat Med 2, 1035-1037

81. Esteller, M., Sanchez-Cespedes, M., Rosell, R., Sidransky, D., Baylin, S. B., and Herman, J. G. (1999) Cancer Res 59, 67-70

82. Mulcahy, H. E., Lyautey, J., Lederrey, C., qi Chen, X., Anker, P., Alstead, E. M., Ballinger, A., Farthing, M. J., and Stroun, M. (1998) Clin Cancer Res 4, 271-275

83. Okamoto, A., Sameshima, Y., Yokoyama, S., Terashima, Y., Sugimura, T., Terada, M., and Yokota, J. (1991) Cancer Res 51, 5171-5176

84. Kohler, M. F., Kerns, B. J., Humphrey, P. A., Marks, J. R., Bast, R. C., Jr., and Berchuck, A. (1993) Obstet Gynecol 81, 643-650

85. Hibi, K., Robinson, C. R., Booker, S., Wu, L., Hamilton, S. R., Sidransky, D., and Jen, J. (1998) Cancer Res 58, 1405-1407

86. Silva, J. M., Dominguez, G., Garcia, J. M., Gonzalez, R., Villanueva, M. J., Navarro, F., Provencio, M., San Martin, S., Espana, P., and Bonilla, F. (1999) Cancer Res 59, 3251-3256

87. Swisher, E. M., Wollan, M., Mahtani, S. M., Willner, J. B., Garcia, R., Goff, B. A., and King, M. C. (2005) Am J Obstet Gynecol 193, 662-667

88. Bottari, P., Aebersold, R., Turecek, F., and Gelb, M. H. (2004) Bioconjug Chem 15, 380-388

89. Lu, Y., Bottari, P., Turecek, F., Aebersold, R., and Gelb, M. H. (2004) Anal Chem 76, 4104-4111

90. Desiere, F., Deutsch, E. W., Nesvizhskii, A. I., Mallick, P., King, N. L., Eng, J. K., Aderem, A., Boyle, R., Brunner, E., Donohoe, S., Fausto, N., Hafen, E., Hood, L., Katze, M. G., Kennedy, K. A., Kregenow, F., Lee, H., Lin, B., Martin, D., Ranish, J. A., Rawlings, D. J., Samelson, L. E., Shiio, Y., Watts, J. D., Wollscheid, B., Wright, M. E., Yan, W., Yang, L., Yi, E. C., Zhang, H., and Aebersold, R. (2005) Genome Biol 6, R9

91. Deutsch, E. W., Eng, J. K., Zhang, H., King, N. L., Nesvizhskii, A. I., Lin, B., Lee, H., Yi, E. C., Ossola, R., and Aebersold, R. (2005) Proteomics 5, 3497-3500

92. Pecorelli, S., Benedet, J. L., Creasman, W. T., and Shepherd, J. H. (1999) Int J Gynaecol Obstet 65, 243-249

93. Gygi, S. P., Rist, B., Gerber, S. A., Turecek, F., Gelb, M. H., and Aebersold, R. (1999) Nat Biotechnol 17, 994-999

94. McGuire, W. P., Hoskins, W. J., Brady, M. F., Kucera, P. R., Partridge, E. E., Look, K. Y., Clarke-Pearson, D. L., and Davidson, M. (1996) N Engl J Med 334, 1-6

95. Ozols, R. F., Bundy, B. N., Greer, B. E., Fowler, J. M., Clarke-Pearson, D., Burger, R. A., Mannel, R. S., DeGeest, K., Hartenbach, E. M., and Baergen, R. (2003) J Clin Oncol 21, 3194-3200

96. Gerber, S. A., Rush, J., Stemman, O., Kirschner, M. W., and Gygi, S. P. (2003) Proc Natl Acad Sci USA 100, 6940-6945

97. Rush, J., Moritz, A., Lee, K. A., Guo, A., Goss, V. L., Spek, E. J., Zhang, H., Zha, X. M., Polakiewicz, R. D., and Comb, M. J. (2005) Nat Biotechnol 23, 94-101

98. Zhang, H., Zha, X., Tan, Y., Hornbeck, P. V., Mastrangelo, A. J., Alessi, D. R., Polakiewicz, R. D., and Comb, M. J. (2002) J Biol Chem 277, 39379-39387

99. Anderson, N. L., Anderson, N. G., Haines, L. R., Hardie, D. B., Olafson, R. W., and Pearson, T. W. (2004) J Proteome Res 3, 235-244

All of the U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing it will be appreciated that, although specific embodiments of the invention have been described herein for purposes of illustration, various modifications may be made without deviating from the spirit and scope of the invention. Accordingly, the invention is not limited except as by the appended claims.

What is claimed is:

1. A diagnostic panel comprising:
a plurality of detection reagents wherein each detection reagent is specific for one tissue-derived serum glycoprotein; wherein the tissue-derived serum glycoproteins detected by the plurality of detection reagents are derived from the same tissue and selected from the tissue-derived serum glycoprotein sets provided in Table 1.

2. The diagnostic panel of claim 1 wherein the plurality of detection reagents is selected such that the level of at least two of the tissue-derived serum glycoproteins detected by the plurality of detection reagents in a blood sample from a subject afflicted with a disease affecting a tissue from which the tissue-derived serum glycoproteins are derived is above or below a predetermined normal range.

3. The diagnostic panel of claim 1 wherein the plurality of detection reagents is selected such that the level of at least three of the tissue-derived serum glycoproteins detected by the plurality of detection reagents in a blood sample from a subject afflicted with a disease affecting the organ from which the tissue-derived serum glycoproteins are derived is above or below a predetermined normal range.

4. The diagnostic panel of claim 1 wherein the plurality of detection reagents is selected such that the level of at least four of the tissue-derived serum glycoproteins detected by the plurality of detection reagents in a blood sample from a subject afflicted with a disease affecting the organ from which the tissue-derived serum glycoproteins are derived is above or below a predetermined normal range.

5. The diagnostic panel of claim 1 wherein the plurality of detection reagents is between two and 100 detection reagents.

6. The diagnostic panel of claim 2 wherein the disease affects the prostate and the tissue-derived serum glycoproteins detected by the plurality of detection reagents are selected from the prostate-derived serum glycoproteins listed in Table 1.

7. The diagnostic panel of claim 6 wherein the plurality of detection reagents detect two or more of the prostate-derived serum glycoproteins listed in Table 1.

8. The diagnostic panel of claim 6 wherein the plurality of detection reagents detect three or more of the prostate-derived serum glycoproteins listed in Table 1.

9. The diagnostic panel of claim 6 wherein the plurality of detection reagents detect four or more of the prostate-derived serum glycoproteins listed in Table 1.

10. The diagnostic panel of claim 6 wherein the plurality of detection reagents detect five or more of the prostate-derived serum glycoproteins listed in Table 1.

11. The diagnostic panel of claim 6 wherein the plurality of detection reagents detect two or more prostate-derived serum glycoproteins selected from the group consisting of CD13, CD14, CD26, CD44, CD45, CD56, CD90, CD91, CD107a, CD107b, CD109, CD166, CD143, CD224, PSMA-1, Glutamate carboxypeptidase II, MAC-2 binding protein, metalloproteinase inhibitor 1, and tumor endothelial marker 7-related precursor.

12. The diagnostic panel of claim 6 further comprising one or more detection reagents that are each specific for a prostate-derived glycoprotein listed in Table 1 that does not overlap with the plasma-derived glycoproteins listed in Table 1.

13. The diagnostic panel of claim 2 wherein the disease affects the bladder and the tissue-derived serum glycoproteins detected by the plurality of detection reagents are selected from the bladder-derived serum glycoproteins listed in Table 1.

14. The diagnostic panel of claim 13 wherein the plurality of detection reagents detect two or more of the bladder-derived serum glycoproteins listed in Table 1.

15. The diagnostic panel of claim 13 wherein the plurality of detection reagents detect three or more of the bladder-derived serum glycoproteins listed in Table 1.

16. The diagnostic panel of claim 13 wherein the plurality of detection reagents detect four or more of the bladder-derived serum glycoproteins listed in Table 1.

17. The diagnostic panel of claim 13 wherein the plurality of detection reagents detect five or more of the bladder-derived serum glycoproteins listed in Table 1.

18. The diagnostic panel of claim 13 further comprising one or more detection reagents that are each specific for a bladder-derived glycoprotein listed in Table 1 that does not overlap with the plasma-derived glycoproteins listed in Table 1.

19. The diagnostic panel of claim 1 wherein the disease affects the liver and the tissue-derived serum glycoproteins detected by the plurality of detection reagents are selected from the liver-derived serum glycoproteins listed in Table 1.

20. The diagnostic panel of claim 19 wherein the plurality of detection reagents detect two or more of the liver-derived serum glycoproteins listed in Table 1.

21. The diagnostic panel of claim 19 wherein the plurality of detection reagents detect three or more of the liver-derived serum glycoproteins listed in Table 1.

22. The diagnostic panel of claim 19 wherein the plurality of detection reagents detect four or more of the liver-derived serum glycoproteins listed in Table 1.

23. The diagnostic panel of claim 19 wherein the plurality of detection reagents detect five or more of the liver-derived serum glycoproteins listed in Table 1.

24. The diagnostic panel of claim 19 further comprising one or more detection reagents that are each specific for a liver-derived glycoprotein listed in Table 1 that does not overlap with the plasma-derived glycoproteins listed in Table 1.

25. The diagnostic panel of claim 2 wherein the disease affects the breast and the tissue-derived serum glycoproteins detected by the plurality of detection reagents are selected from the breast-derived serum glycoproteins listed in Table 1.

26. The diagnostic panel of claim 25 wherein the plurality of detection reagents detect two or more of the breast-derived serum glycoproteins listed in Table 1.

27. The diagnostic panel of claim 25 wherein the plurality of detection reagents detect three or more of the breast-derived serum glycoproteins listed in Table 1.

28. The diagnostic panel of claim 25 wherein the plurality of detection reagents detect four or more of the breast-derived serum glycoproteins listed in Table 1.

29. The diagnostic panel of claim 25 wherein the plurality of detection reagents detect five or more of the breast-derived serum glycoproteins listed in Table 1.

30. The diagnostic panel of claim 25 wherein the plurality of detection reagents detect two or more breast-derived serum glycoproteins selected from the group consisting of CD71, CD98, CD107b, CD155, CD224, MAC-2 binding protein, receptor protein-tyrosine kinase erbB-2, and tumor-associated calcium signal transducer 2.

31. The diagnostic panel of claim 25 further comprising one or more detection reagents that are each specific for a breast-derived glycoprotein listed in Table 1 that does not overlap with the plasma-derived glycoproteins listed in Table 1.

32. The diagnostic panel of claim 2 wherein the disease affects lymphocytes and the tissue-derived serum glycoproteins detected by the plurality of detection reagents are selected from the lymphocyte-derived serum glycoproteins listed in Table 1.

33. The diagnostic panel of claim 32 wherein the plurality of detection reagents detect two or more of the lymphocyte-derived serum glycoproteins listed in Table 1.

34. The diagnostic panel of claim 32 wherein the plurality of detection reagents detect three or more of the lymphocyte-derived serum glycoproteins listed in Table 1.

35. The diagnostic panel of claim 32 wherein the plurality of detection reagents detect four or more of the lymphocyte-derived serum glycoproteins listed in Table 1.

36. The diagnostic panel of claim 32 wherein the plurality of detection reagents detect five or more of the lymphocyte-derived serum glycoproteins listed in Table 1.

37. The diagnostic panel of claim 32 further comprising one or more detection reagents that are each specific for a lymphocyte-derived glycoprotein listed in Table 1 that does not overlap with the plasma-derived glycoproteins listed in Table 1.

38. The diagnostic panel of claim 2 wherein the disease affects the ovary and the tissue-derived serum glycoproteins detected by the plurality of detection reagents are selected from the ovary-derived serum glycoproteins listed in Table 1.

39. The diagnostic panel of claim 38 wherein the plurality of detection reagents detect two or more of the ovary-derived serum glycoproteins listed in Table 1.

40. The diagnostic panel of claim 38 wherein the plurality of detection reagents detect three or more of the ovary-derived serum glycoproteins listed in Table 1.

41. The diagnostic panel of claim 38 wherein the plurality of detection reagents detect four or more of the ovary-derived serum glycoproteins listed in Table 1.

42. The diagnostic panel of claim 38 wherein the plurality of detection reagents detect five or more of the ovary-derived serum glycoproteins listed in Table 1.

43. The diagnostic panel of claim 38 further comprising one or more detection reagents that are each specific for a ovary-derived glycoprotein listed in Table 1 that does not overlap with the plasma-derived glycoproteins listed in Table 1.

44. A diagnostic panel comprising:
a plurality of detection reagents wherein each detection reagent is specific for one tissue-derived serum glycoprotein; wherein the tissue-derived serum glycoproteins detected by the plurality of detection reagents are selected from two or more of the tissue-derived serum glycoprotein sets provided in Table 1.

45. The diagnostic panel of claim 44 wherein the plurality of detection reagents is selected such that the level of at least two of the tissue-derived serum glycoproteins detected by the plurality of detection reagents in a blood sample from a subject afflicted with a disease affecting the organs from which the tissue-derived serum glycoproteins are derived is above or below a predetermined normal range.

46. The diagnostic panel of claim 44 wherein the plurality of detection reagents is selected such that the level of at least three of the tissue-derived serum glycoproteins detected by the plurality of detection reagents in a blood sample from a subject afflicted with a disease affecting the organs from which the tissue-derived serum glycoproteins are derived is above or below a predetermined normal range.

47. The diagnostic panel of claim 44 wherein the plurality of detection reagents is selected such that the level of at least four of the tissue-derived serum glycoproteins detected by the plurality of detection reagents in a blood sample from a subject afflicted with a disease affecting the organs from which the tissue-derived serum glycoproteins are derived is above or below a predetermined normal range.

48. The diagnostic panel of claim 44 wherein the plurality of detection reagents is between two and 100 detection reagents.

49. The diagnostic panel of claim 1 or claim 44 wherein the detection reagent comprises an antibody or an antigen-binding fragment thereof.

50. The diagnostic panel of claim 1 or claim 44 wherein the detection reagent comprises a DNA or RNA aptamer.

51. The diagnostic panel of claim 1 or claim 44 wherein the detection reagent comprises an isotope labeled peptide.

52. A method for defining a biological state of a subject comprising;
a. measuring the level of at least two tissue-derived serum glycoproteins selected from any one of the tissue-derived serum glycoprotein sets provided in Table 1 in a blood sample from the subject;
b. comparing the level determined in (a) to a predetermined normal level of the at least two tissue-derived serum glycoproteins;
wherein the measured level of at least one of the two tissue-derived serum glycoproteins is above or below the predetermined normal level and wherein said measured level defines the biological state of the subject.

53. The method of claim 52, wherein the level of the at least two tissue-derived serum glycoproteins is measured using an immunoassay.

54. The method of claim 53 wherein the immunoassay comprises an ELISA.

55. The method of claim 52 wherein the level of the at least two tissue-derived serum glycoproteins is measured using mass spectrometry.

56. The method of claim 52 wherein the level of the at least two tissue-derived serum glycoproteins is measured using an aptamer capture assay.

57. A method for defining a biological state of a subject comprising;
a. measuring the level of at least two tissue-derived serum glycoproteins selected from any two or more of the tissue-derived serum glycoprotein sets provided in Table 1;
b. comparing the level determined in (a) to a predetermined normal level of the at least two tissue-derived serum glycoproteins;
wherein the measured level of at least one of the two tissue-derived serum glycoproteins is above or below the predetermined normal level and wherein said measured level defines the biological state of the subject.

58. The method of claim 57, wherein the level of the at least two tissue-derived serum glycoproteins is measured using an immunoassay.

59. The method of claim 58 wherein the immunoassay comprises an ELISA.

60. The method of claim 57 wherein the level of the at least two tissue-derived serum glycoproteins is measured using mass spectrometry.

61. The method of claim 57 wherein the level of the at least two tissue-derived serum glycoproteins is measured using an aptamer capture assay.

62. A method for defining a disease-associated tissue-derived blood fingerprint comprising;
a. measuring the level of at least two tissue-derived serum glycoproteins selected from any one of the tissue-derived serum glycoprotein sets provided in Table 1 in a blood sample from a subject determined to have a disease affecting the tissue from which the at least two tissue-derived serum glycoproteins are selected;
b. comparing the level of the at least two tissue-derived serum glycoproteins determined in (a) to a predetermined normal level of the at least two tissue-derived serum glycoproteins;
wherein the measured level of at least one of the at least two tissue-derived serum glycoproteins in the blood sample from the subject determined to have the disease is below or above the corresponding predetermined normal level and wherein said measured level defines the disease-associated tissue-derived blood fingerprint.

63. The method of claim 62 wherein step (a) comprises measuring the level of at least three tissue-derived serum glycoproteins selected from any one of the tissue-derived serum glycoprotein sets provided in Table 1 and wherein the measured level of at least two of the at least three tissue-derived serum glycoproteins in the blood sample from the subject determined to have the disease is below or above the corresponding predetermined normal level and wherein said measured level defines the disease-associated tissue-derived blood fingerprint.

64. The method of claim 62 wherein step (a) comprises measuring the level of four or more tissue-derived serum glycoproteins selected from any one of the tissue-derived serum glycoprotein sets provided in Table 1 and wherein a level of at least three of the four or more tissue-derived serum glycoproteins in the blood sample from the subject determined to have the disease that is below or above the corresponding predetermined normal level defines the disease-associated tissue-derived blood fingerprint.

65. The method of claim 62 wherein step (a) comprises measuring the level of four or more tissue-derived serum glycoproteins selected from any one of the tissue-derived serum glycoprotein sets provided in Table 1 and wherein a level of at least four of the four or more tissue-derived serum glycoproteins in the blood sample from the subject determined to have the disease that is below or above the corresponding predetermined normal level defines the disease-associated tissue-derived blood fingerprint.

66. The method of claim 62 wherein step (a) comprises measuring the level of five or more tissue-derived serum glycoproteins selected from any one of the tissue-derived serum glycoprotein sets provided in Table 1 and wherein a level of at least five of the five or more tissue-derived serum glycoproteins in the blood sample from the subject determined to have the disease that is below or above the corresponding predetermined normal level defines the disease-associated tissue-derived blood fingerprint.

67. The method of claim 62 wherein the level of the at least two tissue-derived serum glycoproteins is measured using antibodies or antigen-binding fragments thereof specific for each protein.

68. The method of claim 67 wherein the antibodies or antigen-binding fragments thereof are monoclonal antibodies.

69. The method of claim 62 wherein the level of the at least two tissue-derived serum glycoproteins is measured using mass spectrometry.

70. The method of claim 62 wherein the level of the at least two tissue-derived serum glycoproteins is measured using an aptamer capture assay.

71. The method of claim 62 wherein the disease is prostate cancer and the at least two tissue-derived serum glycoproteins are selected from the prostate-derived serum glycoproteins listed in Table 1.

72. The method of claim 62 wherein the disease is breast cancer and the at least two tissue-derived serum glycoproteins are selected from the breast-derived serum glycoproteins listed in Table 1.

73. The method of claim 62 wherein the disease is bladder cancer and the at least two tissue-derived serum glycoproteins are selected from the bladder-derived serum glycoproteins listed in Table 1.

74. The method of claim 62 wherein the disease is liver cancer and the at least two tissue-derived serum glycoproteins are selected from the liver-derived serum glycoproteins listed in Table 1.

75. A method for defining a disease-associated tissue-derived blood fingerprint comprising;
a. measuring the level of at least two tissue-derived serum glycoproteins selected from two or more of the tissue-derived serum glycoprotein sets provided in Table 1 in a blood sample from a subject determined to have a disease of interest;
b. comparing the level of the at least two tissue-derived serum glycoproteins determined in (a) to a predetermined normal level of the at least two tissue-derived serum glycoproteins;
wherein a level of at least one of the at least two tissue-derived serum glycoproteins in the blood sample from the subject determined to have the disease that is below or above the corresponding predetermined normal level defines the disease-associated tissue-derived blood fingerprint.

76. The method of claim 75 wherein step (a) comprises measuring the level of at least three tissue-derived serum glycoproteins selected from two or more of the tissue-derived serum glycoprotein sets provided in Table 1 and wherein a level of at least two of the at least three tissue-derived serum glycoproteins in the blood sample from the subject determined to have the disease that is below or above the corresponding predetermined normal level defining the disease-associated tissue-derived blood fingerprint.

77. The method of claim 75 wherein step (a) comprises measuring the level of four or more tissue-derived serum glycoproteins selected from two or more of the tissue-derived serum glycoprotein sets provided in Table 1 and wherein a level of at least three of the four or more tissue-derived serum glycoproteins in the blood sample from the subject determined to have the disease that is below or above the corresponding predetermined normal level defining the disease-associated tissue-derived blood fingerprint.

78. The method of claim 75 wherein step (a) comprises measuring the level of four or more tissue-derived serum glycoproteins selected from two or more of the tissue-derived serum glycoprotein sets provided in Table 1 and wherein a level of at least four of the four or more tissue-derived serum glycoproteins in the blood sample from the subject determined to have the disease that is below or above the corresponding predetermined normal level defining the disease-associated tissue-derived blood fingerprint.

79. The method of claim 75 wherein step (a) comprises measuring the level of five or more tissue-derived serum glycoproteins selected from two or more of the tissue-derived serum glycoprotein sets provided in Table 1 and wherein a level of at least five of the five or more tissue-derived serum glycoproteins in the blood sample from the subject determined to have the disease that is below or above the corresponding predetermined normal level defining the disease-associated tissue-derived blood fingerprint.

80. A method for detecting perturbation of a normal biological state in a subject comprising,
a) contacting a blood sample from the subject with a plurality of detection reagents wherein each detection reagent is specific for one tissue-derived serum glycoprotein; wherein the tissue-derived serum glycoproteins detected by the plurality of detection reagents are selected from any one of the tissue-derived serum glycoprotein sets provided in Table 1;
b) measuring the amount of the tissue-derived serum glycoprotein detected in the blood sample by each detection reagent; and
c) comparing the amount of the tissue-derived serum glycoprotein detected in the blood sample by each detection reagent to a predetermined normal amount for each respective tissue-derived serum glycoprotein;
wherein a statistically significant altered level in one or more of the tissue-derived serum glycoproteins indicates a perturbation in the normal biological state.

81. A method for detecting perturbation of a normal biological state in a subject comprising,
a) contacting a blood sample from the subject with a plurality of detection reagents wherein each detection reagent is specific for one tissue-derived serum glycoprotein; wherein the tissue-derived serum glycoproteins detected by the plurality of detection reagents are selected from two or more of the tissue-derived serum glycoprotein sets provided in Table 1;
b) measuring the amount of the tissue-derived serum glycoprotein detected in the blood sample by each detection reagent; and
c) comparing the amount of the tissue-derived serum glycoprotein detected in the blood sample by each detection reagent to a predetermined normal amount for each respective tissue-derived serum glycoprotein;
wherein a statistically significant altered level in one or more of the tissue-derived serum glycoproteins indicates a perturbation in the normal biological state.

82. A method for detecting prostate disease in a subject comprising,
a) contacting a blood sample from the subject with a plurality of detection reagents wherein each detection reagent is specific for one prostate-derived protein; wherein the prostate-derived proteins are selected from the prostate-derived serum glycoprotein set provided in Table 1;
b) measuring the amount of the tissue-derived serum glycoprotein detected in the blood sample by each detection reagent; and
c) comparing the amount of the tissue-derived serum glycoprotein detected in the blood sample by each detection reagent to a predetermined normal control amount for each respective tissue-derived serum glycoprotein;
wherein a statistically significant altered level in one or more of the tissue-derived serum glycoproteins indicates the presence of prostate disease in the subject.

83. The method of claim 82 wherein the prostate disease is selected from the group consisting of prostate cancer, prostatitis, and benign prostatic hyperplasia.

84. The method of claim 82 wherein the plurality of detection reagents comprises at least 2 detection reagents.

85. The method of claim 82 wherein the plurality of detection reagents comprises at least 3 detection reagents.

86. The method of claim 82 wherein the plurality of detection reagents comprises at least 4 detection reagents.

87. The method of claim 82 wherein the plurality of detection reagents comprises at least 5 detection reagents.

88. The method of claim 82 wherein the plurality of detection reagents comprises at least 6 detection reagents.

89. A method for monitoring a response to a therapy in a subject, comprising the steps of:
(a) measuring in a blood sample obtained from the subject the level of a plurality of tissue-derived serum glycoproteins, wherein the plurality of tissue-derived serum glycoproteins are selected from any one of the tissue-derived serum glycoprotein sets provided in Table 1;
(b) repeating step (a) using a blood sample obtained from the subject after undergoing therapy; and
(c) comparing the level of the plurality of tissue-derived serum glycoproteins detected in step (b) to the amount detected in step (a) and therefrom monitoring the response to the therapy in the patient.

90. A method for monitoring a response to a therapy in a subject, comprising the steps of:
(a) measuring in a blood sample obtained from the subject the level of a plurality of tissue-derived serum glycoproteins, wherein the plurality of tissue-derived serum glycoproteins are selected from two or more of the tissue-derived serum glycoprotein sets provided in Table 1;
(b) repeating step (a) using a blood sample obtained from the subject after undergoing therapy; and
(c) comparing the level of the plurality of tissue-derived serum glycoproteins detected in step (b) to the amount detected in step (a) and therefrom monitoring the response to the therapy in the patient.

91. A targeting agent comprising an tissue-derived probe that specifically recognizes a sequence of any one or more of the sequences set forth in Table 1, wherein said probe has attached thereto a therapeutic agent, said therapeutic agent comprising a radioisotope or cytotoxic agent.

92. An assay device comprising a panel of detection reagents wherein each detection reagent in the panel, with the exception of a negative and positive control, is capable of specific interaction with one of a plurality of tissue-derived serum glycoproteins present in blood, wherein the plurality of tissue-derived serum glycoproteins are derived from the same tissue and wherein the pattern of interaction between the detection reagents and the tissue-derived serum glycoproteins present in a blood sample is indicative of a biological condition.
